(19) United States
(12) Patent Application Publication
(10) Pub. No.: US 2025/0250303 A1
(43) Pub. Date: Aug. 7, 2025

(54) ACTIVATION OF THE INSULIN RECEPTOR BY AN INSULIN MIMETIC PEPTIDE

(71) Applicants: THE TRUSTEES OF COLUMBIA UNIVERSITY IN THE CITY OF NEW YORK, New York, NY (US); THE UNIVERSITY OF TEXAS SOUTHWESTERN MEDICAL CENTER, Dallas, TX (US); UNIVERSITY OF COLORADO, Boulder, CO (US)

(72) Inventors: Eunhee CHOI, Fort Lee, NJ (US); Xiaochen BAI, Dallas, TX (US); Michael STOWELL, Boulder, CO (US)

(21) Appl. No.: 19/087,182

(22) Filed: Mar. 21, 2025

Related U.S. Application Data
(63) Continuation-in-part of application No. PCT/US2023/074946, filed on Sep. 22, 2023.
(60) Provisional application No. 63/409,123, filed on Sep. 22, 2022.

(51) Int. Cl. C07K 14/00; A61K 38/00

(52) U.S. Cl. CPC C07K 14/001; A61K 38/00

(57) ABSTRACT

Compositions and methods for treating insulin resistance, reducing blood glucose level, increasing insulin tolerance, and treating diabetes. The methods include administering the composition to a subject. The subject may express insulin receptor with site-1 binding-deficient mutations or insulin-desensitized insulin receptor. The composition includes a synthetic peptide of 18-35 amino acid residues in length. The composition may include insulin or an analog thereof. The synthetic peptide has a linker region and a component 2 region. The synthetic peptide may also have a component 1 region. A method of activating PI3K-AKT pathway without activating MAPK pathway in a cell includes administering the synthetic peptide to the cell. Uses for the synthetic peptide include the treatment of insulin resistance, the reduction of blood glucose level, increasing insulin tolerance, treating diabetes, and the manufacture of a medicament for these purposes.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### CROSS-REFERENCE TO RELATED APPLICATION

This application claims priority to and is a continuation-in-part of International Application No. PCT/US23/74946, filed on Sep. 22, 2023, which claims the benefit of U.S. Provisional Application No. 63/409,123 filed Sep. 22, 2022, the contents of which is hereby incorporated by reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 50,012-byte SML file named “44010-139_SeqList” created on Mar. 21, 2025.

### BACKGROUND

The insulin receptor (IR) is a receptor tyrosine kinase which upon insulin binding at the cell surface, triggers signaling cascades to control many facets of physiology, such as promoting cellular glucose uptake, glycogen, lipid and protein synthesis, and cell growth. Activated IR phosphorylates itself as well as the IR substrates (IRS) family of proteins and src homology 2 (SHC) proteins, activating PI3K-AKT and MAP kinase (MAPK) pathways. Dysfunctional IR signaling causes metabolic disorders such as type 1 and type 2 diabetes. Over 8 million people worldwide suffer from type 1 diabetes. Insulin therapy is vital for people with type 1 diabetes for maintaining normal glucose levels but can cause side effects and complications such as hypoglycemia and heart disease. Type 2 diabetes is a pandemic of the 21st century, affecting over 400 million people worldwide. Basal insulin therapy is recommended for patients with type 2 diabetes, but around 40% of patients fail to achieve their glycated hemoglobin targets with basal insulin therapy. Insulin regimens often become ineffective in ensuring blood glucose control and they often increase the risk of side effects such as weight gain. The rising prevalence of diabetes and the insufficiency of established therapeutic methods mandate the development of new strategies to complement existing approaches.

Mutations in the IR cause severe insulin resistance, such as with Donohue syndrome and Rabson-Mendenhall syndrome. These patients have mutations in the IR which abrogates normal IR signaling and clinically results in severe growth deficiencies and death within the first decade of life. While insulin, insulin sensitizers, leptin and insulin-like growth factor 1 (IGF1) can temporarily achieve glycemic control and alleviate the symptoms of these complications, these approaches do not restore normal IR signaling. Further, there is no specific long-term treatment for patients with severe insulin resistance diseases where their life expectancy is only a few years. The majority of the disease-causing IR mutations in these syndromes are missense mutations. Some of the IR mutations have been examined for their function and IR signaling; although, the lack of a relevant model to study the pathophysiology of insulin resistance has hampered the ability to investigate potential treatments suitable for translation to clinical settings. In particular, understanding how disease-causing IR mutations affect insulin binding, kinase activation, and trafficking remains largely unclear.

### SUMMARY

In some aspects, a synthetic peptide of 18-35 amino acid residues in length is disclosed. The synthetic peptide comprises a linker region comprising 4-10 amino acid residues; and a component 2 region comprising at least 14 amino acid residues; wherein: the linker region comprises an amino acid sequence with at least 66% sequence identity to SEQ ID NO. 2; and the component 2 region comprises an amino acid sequence with at least 85% sequence identity to SEQ ID NO. 1. In some aspects, the synthetic peptide has the amino acid sequence set forth in SEQ ID NO. 45. In particular embodiments, the amino acid sequence of the linker region is selected from the group consisting of: SEQ ID NOs. 3-12. In certain implementations, the amino acid sequence of the component 2 region has at least 92% sequence identity to SEQ ID NO. 1. In particular embodiments, the amino acid sequence of the component 2 region is selected from the group consisting of SEQ ID NO. 1 and 15-21. In some aspects, the synthetic peptide further comprises a component 1 region comprising at least 11 amino acid residues with at least at least 90% sequence identity to SEQ ID NO. 3. In particular embodiments, the amino acid sequence of the component 1 region is set forth in SEQ ID NO. 3 or SEQ ID NO. 22. In certain implementations, the synthetic peptide is selected from SEQ ID NOs. 23-36. In particular embodiments, the synthetic peptide further comprises an extension of 1-5 amino acid residues at the N terminus. In some aspects, the synthetic peptide further comprises a lipidation modification. In certain implementations, the synthetic peptide is SEQ ID NO. 39. In some embodiments, at least one glycine, cysteine, or serine residue of the synthetic peptide has the lipidation modification. In some aspects, the synthetic peptide is selected from SEQ ID NOs. 40-44.

In another aspect, a therapeutic composition is disclosed. The therapeutic composition comprises insulin or analog thereof; and any one of the synthetic peptides disclosed herein. In particular embodiments, a method of treating insulin resistance in a subject is disclosed. The method comprises administering to the subject any one of the synthetic peptides disclosed herein. In certain implementations, the subject expresses insulin receptor with site-1 binding-deficient mutations. In some aspects, the subject expresses insulin-desensitized insulin receptor. Use of any one of the synthetic peptides disclosed herein for the treatment of insulin resistance is disclosed. Use of any one of the synthetic peptides disclosed herein for the manufacture of a medicament to treat insulin resistance is disclosed. In particular embodiments, the insulin resistance is caused by insulin receptor with site-1 binding-deficient mutations or insulin-desensitized insulin receptor.

In another aspect, a method of reducing blood glucose level in a subject is disclosed. The method comprises administering to the subject any one of the synthetic peptides disclosed herein. In some implementations, the subject is diagnosed with cancer. Use of any one of the synthetic peptides disclosed herein for the reduction of blood glucose level is disclosed. Use of any one of the synthetic peptides disclosed herein for the manufacture of a medicament to reduce blood glucose level is disclosed.

In another aspect, a method of increasing insulin tolerance in a subject is disclosed. The method comprises administering to the subject any one of the synthetic peptides disclosed herein. In some implementations, the subject is diagnosed with cancer. Use of any one of the synthetic peptides disclosed herein for increasing of insulin tolerance is disclosed. Use of any one of the synthetic peptides disclosed herein for the manufacture of a medicament to increase insulin tolerance is disclosed.

In another aspect, a method of treating diabetes in a subject is disclosed. The method comprises administering to the subject any one of the synthetic peptides disclosed herein. In some implementations, the subject is diagnosed with cancer. In certain implementations, the method further comprises administering to the subject insulin or analog thereof. Use of any one of the synthetic peptides disclosed herein for treating diabetes is disclosed. Use of any one of the synthetic peptides disclosed herein for the manufacture of a medicament to treat diabetes is disclosed.

In particular embodiments, the medicaments disclosed herein further comprise insulin or an analog thereof.

In another aspect, a method of activating PI3K-AKT pathway without activating MAPK pathway in a cell is disclosed. The method comprises administering to the cell any one of the synthetic peptides disclosed herein.

### BRIEF DESCRIPTION OF FIGURES

The patent or application file contains at least one drawing executed in color. Copies of this patent or patent application publication with color drawing(s) will be provided by the Office upon request and payment of the necessary fec.

FIGS. 1A-1E depict two distinct insulin binding sites of the IR synergistically induce optimal receptor activation. FIG. 1A illustrates the structure of IR with insulin bound only at site-2 (A-shape). FIG. 1B shows the structure of IR with insulin bound only at site-1 (Asymmetric conformation). FIG. 1C illustrates the structure of IR with insulin bound at both site-1 and site-2 (T-shape). FIG. 1D is a model of active IR/insulin complex and FIG. 1E is a model of active IR/insulin mimetics complex.

FIGS. 2A-2G depict the overall structure of the IR/S597-component-2 complex. FIG. 2A shows the sequences of S597 and S597-N20. The residues in S597-component-1 are marked in yellow, S597-component-2 in pink, and linker in black. The disulfide bond was indicated as red. FIG. 2B illustrates the 3D reconstruction of the IR dimer with two S597-N20 peptides bound. FIG. 2C shows the ribbon representation of the IR dimer with two S597-N20 peptides bound fitted into cryo-EM map at 3.6 Å resolution. FIG. 2D depicts 3D reconstructions of the IR dimer from the gray dashed line in FIG. 2B after forced 3D refinement. FIG. 2E shows the ribbon representations of the IR dimer from the gray dashed line in FIG. 2B after forced 3D refinement, fitted into cryo-EM map at 3.5 Å resolution. FIG. 2F is a close-up view of the S597-N20. FIG. 2G is a close-up view of the binding of S597-N20 (pink) at the FnIII-1 domain of IR (green).

FIGS. 3A-3J depict how S597 binds to the L1 and FnIII-1 domains of IR. FIG. 3A shows the sequences of S597, S597-W6A, S597-F27A, S597-S20D, S597-N20, S519-C16 and S519. The residues in component-1 are marked in yellow, component-2 in pink, mutation in green, and linker in black. The disulfide bond was indicated as red. FIG. 3B is a close-up view of the binding of S597-component-1 (yellow) at the L1 domain of IR (blue). PDB: 5J3H. FIG. 3C is a close-up view of the binding of insulin (yellow) at the L1/α-CT domains of IR (blue). PDB: 6PXV FIG. 3D depicts the sequence alignment of human (Hs) IR, S597, S519 and S597-S20D. FIG. 3E is a close-up view of the binding of S597-component-2 (pink) at the FnIII-1 domain of IR (green). FIG. 3F is a close-up view of the binding of insulin (pink) at the FnIII-1 domain of IR (green). PDB: 6PXV. FIG. 3G depicts the auto-phosphorylation of IR (pY IR) by 10 nM insulin or S597 for 10 min in 293 FT cells expressing wild-type (WT) IR or the indicated IR mutants. FIG. 3H is a quantification of the western blot data shown in FIG. 3G. Mean±SD. Levels of pY IR were normalized to total IR levels and shown as intensities relative to that of IR WT in insulin-treated cells. Each experiment was repeated four times. Significance calculated using two-tailed Student's t-test; between WT and mutants, **p<0.01 and *p<0.0001. The exact p values are provided in the source data. FIG. 3I depicts the auto-phosphorylation of IR by 10 nM insulin or S597 analogs for 10 min in 293 FT cells expressing IR WT. FIG. 3J is a quantification of the western blot data shown in FIG. 3I. Mean±SD. Levels of pY IR were normalized to total IR levels and shown as intensities relative to that of IR WT in S597-treated cells. Each experiment was repeated three times. Significance calculated using two-tailed Student's t-test. Source data are provided as a Source Data file.

FIGS. 4A-4F depict the overall structure of the IR/S597 complex. FIG. 4A shows a 3D reconstruction of the IR dimer with two S597 peptides bound. FIG. 4B illustrates the ribbon representation of the IR dimer with two S597 peptides bound fitted into cryo-EM map at 5.4 Å resolution. FIG. 4C is a close-up view of the binding of S597 at the L1 domain of one protomer (blue) and FnIII-1 domain of another (green). The component-1 binding helix of S597 is shown in yellow, the component-2 binding helix in pink, and the linker region in gray. FIG. 4D is a superposition between S597-bound IR protomer (blue) and apo-IR protomer (gray). FIG. 4E illustrates a working model for S597-induced IR activation. The simultaneous binding of S597 to both L1 domain of one protomer and FnIII-1 domain of another would trigger structural transition of IR directly from A-shaped apo-form to extended T-shaped IR dimer and stabilize the active form. A cartoon representation of the IR/insulin complex in a compact T-shape is shown for comparison. FIG. 4F shows that the binding of S597-ΔGly to apo-IR would trigger the structural rearrangement of IR from ‘Λ’-shape to ‘extended T’-shape. Such conformational change could be achieved by a simple scissors-like rotation between the two protomers.

FIGS. 5A-5F depict that S597 activates IR mutants that cause severe insulin resistance syndromes. FIG. 5A is an overall view of IR (gray)/insulin (orange) complex in compact T-shape. The IR mutations tested are indicated by red space filling. FIG. 5B is an overall view of IR (gray)/S597 (orange) complex in extended T-shape. The IR mutations tested are indicated by red space filling, except for E697, as the α-CT in the IR/S597 complex could not be observed. FIG. 5C shows the auto-phosphorylation of IR by the indicated concentrations of insulin or S597 for 10 min in 293 FT cells expressing IR wild-type (WT) or indicated disease-causing mutants. FIG. 5D is a quantification of the western blot data shown in FIG. 5C. Levels of pY IR were normalized to total IR levels and shown as intensities relative to that of IR WT in 10 nM insulin-treated cells. Mean±SD. Each experiment was repeated three times. Significance calculated using two-tailed Student's t-test; *p<0.05, **p<0.01, and ***p<0.001. The exact p values are provided in the source data. FIG. 5E shows the auto-phosphorylation of IR by 10 nM insulin or S597 for 10 min in 293 FT cells expressing IR WT or indicated disease-causing mutants. FIG. 5F is a quantification of the western blot data shown in FIG. 5E. Levels of pY IR were normalized to total IR levels and shown as intensities relative to that of IR WT in insulin-treated cells. Mean±SD. Each experiment was repeated three times. Significance calculated using two-tailed Student's t-test; **p<0.01 and ***p<0.001. The exact p values are provided in the source data. Source data are provided as a Source Data file.

FIGS. 6A-6C depict that insulin mimetics selectively activate IR signaling. FIG. 6A illustrates sequences of S597 and analogs. S597 exhibits a helix-loop-helix fold and has a disulfide bond (red line). FIG. 6B shows data related to mice that were injected with PBS, insulin (6 nmol/mouse) or S597 analogs (9 nmol) via inferior vena cava (IVC). Liver (3 min) and skeletal muscle (5 min) were collected after injection. Quantification of data shown in Mean±SD. PBS, N=6 mice; insulin, N=5; S597, N=10; S597-S20D, N=6, S597-ΔGly, N=3. Two-tailed student t-test; *p<0.05; **p<0.01; ***p<0.001; ****p<0.0001 (PBS set as the control); ††p<0.01 and ††††p<0.0001. FIG. 6C shows the mass spectrometry-based phosphoproteomics characterization of IR signaling in skeletal muscle after PBS, insulin or S597 injections. Signaling map showing differentially regulated phosphosites by insulin and S597 (p<0.05, N=3 each). Compared to insulin-treated samples, blue and red sites indicate downregulation and upregulation of phosphorylation by S597, respectively.

FIGS. 7A-7D depict that S597 activates IR signaling in cells expressing disease causing, insulin binding-deficient IR mutants. FIG. 7A shows IR signaling in IR-D707A MEFs treated with the indicated concentrations of insulin or S597 for 10 min. Cell lysates were blotted with the indicated antibodies. 4OHT, 4-Hydroxytamoxifen. FIG. 7B shows IR signaling in IR-D707A MEFs treated with 10 nM insulin or S597 for the indicated times. 4OHT, 4-Hydroxytamoxifen. FIG. 7C illustrates quantification of the western blot data shown in FIG. 7A. Levels of protein phosphorylation were normalized to total protein levels and shown as intensities relative to that in 10 nM S597-treated cells. Mean±SD. Each experiment was repeated four times. Significance calculated using two-tailed Student's t-test; between insulin and S597 in the indicated concentrations, **p<0.01 and ****p<0.0001. The exact p values are provided in the source data. FIG. 7D illustrates quantification of the western blot data shown in FIG. 7B. Levels of protein phosphorylation were normalized to total protein levels and shown as intensities relative to that in 10 nM S597-treated cells for 10 min. Mean±SD. Each experiment was repeated four times. Significance calculated using two-tailed Student's t-test; between insulin and S597 in the indicated time points, **p<0.01, ***p<0.001 and ****p<0.0001. The exact p values are provided in the source data. Source data are provided as a Source Data file.

FIGS. 8A-8G depict that S597 activates IR signaling in vivo. FIG. 8A shows sequences of S597 and S597-S20D. The residues in component-1 are marked in yellow, component-2 in pink, mutation in green, and linker in black. The disulfide bond was indicated as red. FIG. 8B illustrates representative western blots of liver, skeletal muscle, and white adipose tissue (WAT) lysates treated without or with insulin, S597 or S597-S20D. Each lane contains lysate from an individual mouse. FIG. 8C is a quantification of the western blot data shown in FIG. 8B. Levels of protein phosphorylation were normalized to total protein levels and shown as intensities relative to that in insulin-treated conditions. Mean±SD. PBS, n=6 mice; insulin, n=5; S597, n=10; S597-S20D, n=6 for liver and skeletal muscle. PBS, n=5 mice; insulin, n=6; S597, n=7; S597-S20D, n=6 for WAT. Significance calculated using two-tailed Student's t-test; *p<0.05; **p<0.01, ***p<0.001, and ****p<0.0001, with the PBS set as the control. ††p<0.01 and ††††p<0.0001. The exact p values are provided in the source data. FIG. 8D depicts liver sections of mice injected without or with insulin or S597 analogs that were stainedwith anti-IR (Red) antibodies and DAPI (blue). Scale bar, 10 μm. FIG. 8E is a quantification of insulin or S597-dependent nuclear translocation of SREBP1 in primary mouse hepatocytes. Representative images were shown in FIG. 30E. Mean±SD. PBS, n=115; Insulin, n=221; S597, n=217. Significance calculated using two-tailed Student's t-test; ****p<0.0001, with the PBS set as the control. Source data are provided as a Source Data file. FIG. 8F depicts mice that were injected intraperitoneally with Humulin (6 nmol/kg), S597, -W6A-F27A (9 nmol/kg) or -N20 (27 nmol/kg) and FIG. 8G depicts mice that were injected intraperitoneally with S597 or S597-S20D (10 nmol/kg). Mean±SEM, N=8 each. *p<0.05, **p<0.01, ***p<0.001. Two-way ANOVA followed by Tukey's multiple comparisons test.

FIGS. 9A-9C depicts that modes sequence changes modulate signaling selectivity of S597 agonist action. FIG. 9A shows a proposed model of insulin- or S597-induced IR signaling. FIG. 9B depicts liver sections injected with insulin or mimetics stained with anti-IR and DAPI. Quantification of the ratios of plasma membrane (PM) and intracellular (IC) IR signals of the livers was shown. Mean±SD; N=3 mice. FIG. 9C shows a competition assay for full-length IGF1R and IGF1 or S597 analogs. Mean±SD; N=2.

FIGS. 10A and 10B depict that S597 analogs lower blood glucose but not cell proliferation as efficient as insulin. FIG. 10A is an insulin tolerance test (ITT, 3-month-old, male). Mice were injected intraperitoneally with Humulin (6 nmol/kg), S597, -S20D, -ΔGly (10 mol/kg), or S597-N20 (27 nmol/kg). Glucose area under the curve (AUC) during ITT (right). Mean±SEM, *p<0.05, **p<0.01, ***p<0.001. Two-way ANOVA followed by Tukey's multiple comparisons test. Humulin and S597, -S20D, -ΔGly (N=7), and S597-N20 (N=8). FIG. 10B is a MTT assay in C2C12 cells upon insulin, S597, IGF1 or IGF2 stimulation (N=6). Mean±SD. two-tailed Student's t-test.

FIGS. 11A-11F depict that insulin and insulin mimetics regulate metabolism differently in vivo. 2-month-old male mice were implanted with osmotic minipump releasing Humulin (0.2 U/day), S597 (0.3 U/day), or S597-S20D (0.3 U/day). FIG. 11A shows the serum C-peptide level in 4 h fasted mice. Hepes, N=7; Humulin, N=8; S597, N=7; S597-S20D, N=4. FIG. 11B shows the serum insulin level in 4 h fasted mice. Hepes, N=7; Humulin, N=8; S597, N=9; S597-S20D, N=4. Two-way ANOVA followed by Tukey's multiple comparisons test. FIG. 11C shows a GTT. Mean±SEM. Hepes, N=8; Humulin, N=7; S597, N=9; S597-S20D, N=5. FIG. 11D shows the glucose AUC in FIG. 11C. FIG. 11E shows the body weight and FIG. 11F shows the epididymal white adipose tissue mass. Hepes, N=4; Humulin and S597 analogs, N=5. two-tailed Student's t-test. All graphs show Mean±SEM.

FIGS. 12A-12D depict that S597 lowers blood glucose levels in mice. FIG. 12A shows S597 analogs including the sequence of S597 (top) and a subset of the initial structure-based sequence design mutations and extension (XXX) modifications. FIG. 12B illustrates an insulin tolerance test of three-month old male mice. Mice were injected intraperitoneally with Humulin (6 nmol/kg of body weight), S597 (9 nmol/kg) or S597-S20D (9 nmol/kg), and their blood glucose levels were measured at the indicated time points after injection. Mean±SEM. Humulin, n=7 mice; S597, n=6; S597-S20D, n=7. Significance calculated using two-way ANOVA followed by Tukey's multiple comparisons test. *p<0.05, **p<0.01, and ***p<0.001 with the Humulin set as the control. The exact p values are provided in the source data. FIG. 12C illustrates an insulin tolerance test of three-month old male mice. Mice were injected intraperitoneally with S597 (9 nmol/kg), S597-W6A (9 nmol/kg), S597-F27A (9 nmol/kg) or S597-N20 (27 nmol/kg), and their blood glucose levels were measured at the indicated time points after injection. Mean±SEM. S597, n=13 mice; S597-W6A, n=5; S597-F27A, n=5; S597-N20, n=8. Significance calculated using two-way ANOVA followed by Tukey's multiple comparisons test. *p<0.05, **p<0.01, ***p<0.001, and ****p<0.0001 with the S597 set as the control. The exact p values are provided in the source data. Source data are provided as a Source Data file. FIG. 12D shows an insulin tolerance test (ITT, 2˜3-month-old, male). Mice were injected intraperitoneally with PBS, S597 K32+ (9 nmol/kg), S597-S20D K32+ (9 nmol/kg). Mean±SEM, (N=5˜6).

FIGS. 13A-13C depict the preparation of IR proteins. FIG. 13A is a representative SEC of IR/S597 and the SDS PAGE of the peak fraction (coomassie staining). FIG. 13B is a representative cryo-EM micrograph and class averages of IR/S597. FIG. 13C illustrates that the peak fractions were combined and visualized on SDS-PAGE by Coomassie staining, in the absence or presence of dithiothreitol (DTT). Experiment was repeated ten times.

FIGS. 14A-14D depict various Cryo-EM maps of IR/S597. FIG. 14A is a Cryo-EM map of IR/S597. FIG. 14B is a Cryo-EM map of IR/S597-ΔGly at 4.5 Å resolution. FIG. 14C shows the rigid body fitting of the crystal structure of IR L1/CR domains in complex with the component-1 (PDB ID: 5J3H) to the cryo-EM map of IR/S597-ΔGly. FIG. 14D is a close-up view of the rigid body fitting shown in FIG. 14C.

FIGS. 15A and 15B depict the Cryo-EM structures of IR/S597-S20D and IR/S597ΔGly complexes. FIG. 15A is a 3D reconstruction of the IR with either S597-S20D or S597-ΔGly bound. FIG. 15B shows the cryo-EM densities of the TM domain of IR/S597, IR/S597-S20D and IR/S597-ΔGly.

FIGS. 16A-16C depict an analysis of S597. FIG. 16A shows the structure of S597 bound to the IR, highlighting the components-1 and -2 interacting residues (green and red) connected by the disulfide bonded linker. The corresponding sequence of S597 mimetic with the components-1 (red) and -2 (green) residues highlighted. FIG. 16B is an analytical HPLC of the final folded and purified mimetic. FIG. 16C is a typical Orbi-trap HRMS analysis of the purified peptide with observed and calculated M+H ion.

FIG. 17 depicts the distinct active structure of IR and biased signaling by S597. Phosphorylation levels relative to insulin were noted (mean±SD).

FIG. 18 depicts a research plan summary with the specific aims.

FIG. 19 depicts a schematic representation of insulin mimetics discovery. Criteria (% activation obtained by insulin mimetics compared to maximal stimulation by insulin) is shown.

FIG. 20 depicts proposed studies to determine the structural and molecular basis of the biased IR signaling.

FIGS. 21A and 21B depict a salt bridge formation. FIG. 21A shows the component-1 region near Ser20 of S597 showing IR residues Arg118 and His 144 that form a positive pocket suitable for salt-bridge formation. FIG. 21B shows a side chain modelled mutation S20E showing salt bridge formation as depicted by the dashed lines.

FIGS. 22A-22C depict the potency of S597 analogs. FIG. 22A shows the IR signaling in IR/IGF1R double knockout preadipocytes ectopically expressing human IR-B. Cells were treated with the indicated concentrations of insulin or S597 analogs for 10 min. FIG. 22B shows the IR signaling in primary mouse hepatocytes. Cells were treated with 10 nM of the indicated insulin or S597 for the indicated time. FIG. 22C shows the IR signaling in primary hepatocytes. Cells were treated with the indicated concentrations of insulin or S597 for 10 min.

FIGS. 23A and 23B depict domains of insulin receptor (IR) and preparation of S597 peptides. FIG. 23A shows the domain structure of IR. L1 and L2, leucine rich domains 1 and 2; CR, cysteine rich domain, F1, F2, and F3, fibronectin III (FnIII) domains; TM, transmembrane domain; TK, tyrosine kinase domain; α-CT, C-terminal region of IRα. Red lines indicate disulfide bonds. FIG. 23B illustrates HPLC traces and MS1 spectra for S597 and S597 mutant peptides synthesized and utilized for both functional and structural studies.

FIGS. 24A-24E depict a Cryo-EM analysis of the IR/S597-N20 complex. FIG. 24A shows a representative electron micrograph and 2D class averages of the IR/S597-N20 complex. Scale bar: 300 Å. 8388 images were collected. FIG. 24B is an unsharpened cryo-EM map colored by local resolution. FIG. 24C shows the gold-standard Fourier Shell Correlation (FSC) curve for the cryo-EM map shown in FIG. 2. FIG. 24D shows an angular distribution of particles used in the final 3D reconstructions. FIG. 24E is a flowchart of cryo-EM data processing.

FIGS. 25A and 25B depict the Cryo-EM densities of the IR/S597-component-2 and IR/S597 complex. FIG. 25A shows the representative densities of the cryo-EM map of each domain of IR and S597-component-2. FIG. 25B shows the representative densities of the cryo-EM map of one protomer of IR and S597.

FIG. 26 depicts the sequence alignment of IR proteins from human (Hs), mouse (Mn), and Hs IGF1R. IR-A and IR-B indicate A isoform (short) and B isoform (long) of the IR, respectively. Key residues are marked.

FIGS. 27A-27C depict S597-induced IR activation. FIG. 27A shows the IR signaling by the indicated concentrations of insulin or S597 for 10 min in 293 FT cells expressing IR-A or IR-B. FIG. 27B is a quantification of the western blot data shown in FIG. 27A. Levels of protein phosphorylation were normalized to total protein levels and shown as intensities relative to IR-A in 100 nM insulin-treated cells. Mean±SEM. Each experiment was repeated three times. Significance calculated using two-tailed Student's t-test; IR-A between insulin and S597 in the indicated concentrations with the IR-A set as the control. **p<0.01. The exact p values are provided in the source data. FIG. 27C shows that the α-CT motif of IR is not necessary for S597-dependent IR activation. Auto-phosphorylation of IR by the indicated concentrations of insulin or S597 for 10 min in 293 FT cells expressing IR wild-type (WT) or F701A/F705A mutant. Quantification of the western blot data is shown in FIG. 5D. Source data are provided as a Source Data file.

FIGS. 28A-28E depict a Cryo-EM analysis of the IR/S597 complex. FIG. 28A shows a representative electron micrograph and 2D class averages of the IR/S597 complex. Scale bar: 300 Å. 4615 images were collected. FIG. 28B is an unsharpened cryo-EM map colored by local resolution. FIG. 28C shows the gold-standard Fourier Shell Correlation (FSC) curve for the cryo-EM map shown in FIG. 4. FIG. 28D shows an angular distribution of particles used in the final 3D reconstructions. FIG. 28E is a flowchart of cryo-EM data processing.

FIGS. 29A-29D depict the characterization of the tamoxifen-inducible IR knockout mouse embryonic fibroblasts expressing only GFP (GFP MEFs). FIG. 29A shows IR signaling in GFP MEFs treated with the indicated concentrations of insulin or S597 for 10 min. Cell lysates were blotted with the indicated antibodies. 4OHT, 4-Hydroxytamoxifen. FIG. 29B shows IR signaling in GFP MEFs treated with 10 nM insulin or S597 for the indicated times. 4OHT, 4-Hydroxytamoxifen. FIG. 20C is a quantification of the western blot data shown in FIG. 29A. Levels of protein phosphorylation were normalized to total protein levels and shown as intensities relative to that in 10 nM S597-treated cells. Mean±SD. Each experiment was repeated five times. Significance calculated using two-tailed Student's t-test; between insulin and S597 in the indicated concentrations, *p<0.05. The exact p values are provided in the source data. FIG. 29D is a quantification of the western blot data shown in FIG. 29B. Levels of protein phosphorylation were normalized to total protein levels and shown as intensities relative to that in 10 nM S597-treated cells for 10 min. Mean±SD. Each experiment was repeated three times. Significance calculated using two-tailed Student's t-test; between insulin and S597 in the indicated time points, *p<0.05, **p<0.01, and ***p<0.001. The exact p values are provided in the source data. Source data are provided as a Source Data file.

FIGS. 30A-30E depict the action of S597 in IR signaling and SREBP1 activation. FIG. 30A shows IR signaling in primary mouse hepatocytes treated with the indicated concentrations of insulin, S597 or S597-N20 for 10 min. Cell lysates were blotted with the indicated antibodies. FIG. 30B is a quantification of the western blot data shown in FIG. 30A. Levels of protein phosphorylation were normalized to total protein levels and shown as intensities relative to that in 10 nM insulin-treated cells. Mean±SD. For insulin, n=5 independent experiments; S597, n=3; S597-N20, n=2. Significance calculated using two-tailed Student's t-test with the insulin set as the control. *p<0.05; **p<0.01, ***p<0.001, and *p<0.0001. FIG. 30C shows IR signaling in primary mouse hepatocytes treated with 10 nM insulin or S597 for the indicated times. Cell lysates were blotted with the indicated antibodies. FIG. 30D is a quantification of the western blot data shown in FIG. 30C. Levels of protein phosphorylation were normalized to total protein levels and shown as intensities relative to that in 10 nM insulin treated cells for 10 min. Mean±SD. For pY/IR, insulin, n=4 independent experiments and S597, n=3; for pAKT/AKT, insulin, n=6 and S597, n=4; for pERK/ERK, insulin, n=4 and S597, n=3. Significance calculated using two-tailed Student's t-test with the insulin set as the control. *p<0.05. FIG. 30E illustrates insulin- or S597-mediated nuclear translocation of SREBP1 in primary mouse hepatocytes. Cell and nuclear boundaries were noted as white dashed lines. Control (PBS), n=115; Insulin, n=221; S597, n=217. Scale bar, 10 μm. Source data are provided as a Source Data file.

FIGS. 31A-31F depict the effects of chronic treatment with S597. FIG. 31A is a schematic representation of the S597 treatment. Male C57BL/6J mice were treated with vehicle (PBS), insulin (50 U/kg body weight), or S597 (50 U/kg) once daily for three days. OGTT, oral glucose tolerance test; SAC, sacrifice. N=5 mice each. FIG. 31B shows the body weights measured before OGTT. Mean±SEM. N=5 mice each. Significance calculated using two-tailed Student's t-test. No significant. FIG. 31C depicts an oral glucose tolerance test (2 g glucose/kg body weight) after 6 hr. fast. Mean±SEM. N=5 mice each. Significance calculated using two-way ANOVA followed by Tukey's multiple comparisons test. Between PBS and S597, *p<0.05. The exact p values are provided in the source data. FIG. 31D shows the area under curve (AUC) for blood glucose during OGTT depicted in FIG. 31C. Mean±SEM. N=5 mice each. Significance calculated using two-tailed Student's t-test. No significant. FIG. 31E illustrates western blots of liver lysates at the completion of OGTT. Each lane contains lysate from an individual mouse. N=5 mice each. FIG. 31F depicts results when male C57BL/6J mice were fasted for 14 hr. and then injected with vehicle (PBS), insulin (0.1 U/kg), S597 (0.15 U/kg), or S597-S20D (0.15 U/kg). One hour later livers were collected. Western blots of liver lysates are shown. Each lane contains lysate from an individual mouse. N=1 mouse each. Source data are provided as a Source Data file.

FIG. 32 depicts western blots IR signaling induced by insulin and S597 in MCF7, T47D, and CAL33 cells. Cells were treated with a control solution (no ligand), insulin (10 nM, or 100 nM) or S597 (10 nM, 100 nM, or 300 nM) for 10 minutes.

FIG. 33 depicts a graph of comparing the effect of insulin, S597 or IGF1 on MCF7 cell proliferation and growth. Abbreviations in X-axis: 0, no ligand; 110, 10 nM insulin; 1100, 100 nM insulin; S10, 10 nM S597; S100, 100 nM S597; S300, 300 nM S597; IGF1 WT, 10 nM IGF1.

### DETAILED DESCRIPTION

Detailed aspects and applications of the disclosure are described below in the drawings and detailed description of the disclosure. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that the present disclosure may be practiced without these specific details. It should be noted that there are many different and alternative configurations, devices, and technologies to which the disclosed disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

As used herein, the term “% sequence identity” refers to the percentage of residues that match exactly between two different sequences. In other words, “% sequence identity” refers to the ratio of the number of matching residues to the total length of the alignment.

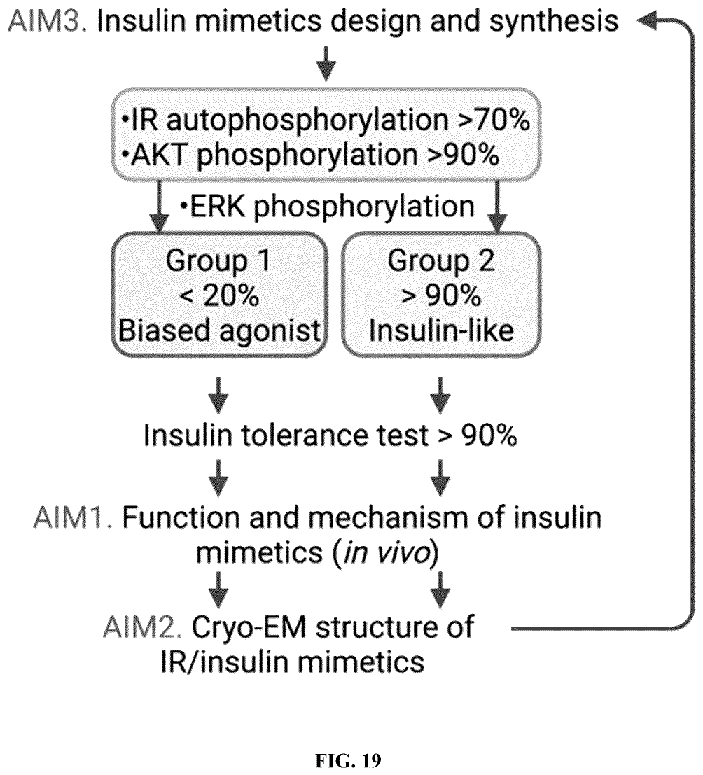

As used herein, the term “biased signaling” refers to activation of downstream insulin receptor (IR) where over 90% of AKT is phosphorylated while less than 20% of ERK is phosphorylated. As such a “biased agonist” is an insulin mimetic that, upon binding to IR, results in phosphorylation of over 90% of the AKT and less than 20% of the ERK (see FIG. 19).

As used herein, the term “insulin-like” refers to IR binding leading to over 90% AKT phosphorylation and over 90% ERK phosphorylation (see FIG. 19).

Disclosed herein is a synthetic peptide of 18-35 amino acid residues in length based on S597, a previously reported selective IR agonist without sequence homology to insulin that activates IR and mimics insulin's action on glycemic control. IR signaling defects cause a variety of metabolic diseases including diabetes. Moreover, inherited mutations of the IR cause severe insulin resistance, leading to early morbidity and mortality with limited therapeutic options. To elucidate the mechanism of IR activation by S597, cryo-EM structures of the mouse IR/S597 complex were determined and novel synthetic peptides that are insulin mimetics were developed.

The synthetic peptide described herein comprises a linker region and at least one region selected from a component 2 region and a component 1 region. The linker region comprises 4-10 amino acid residues. In particular embodiments, the linker region comprises an amino acid sequence with at least 66% sequence identity to SEQ ID NO. 2. In embodiments with a component 2 region, the component 2 region may comprise at least 14 amino acid residues. In particular embodiments, the component 2 region comprises an amino acid sequence with at least 85% sequence identity to SEQ ID NO. 1.

The synthetic peptide may have the amino acid sequence set forth in SEQ ID NO. 45. The amino acid sequence of the linker region may be selected from the group consisting of SEQ ID NOs. 3-14. In particular embodiments, the amino acid sequence of the linker region is selected from the group consisting of SEQ ID NOs. 3-12. The amino acid sequence of the component 2 region may have at least 92% sequence identity to SEQ ID NO. 1. The amino acid sequence of the component 2 region may be selected from the group consisting of SEQ ID NO. 1 and 15-21.

As mentioned above, the synthetic peptide may also comprise a component 1 region. In such embodiments, the component 1 region may comprise at least 11 amino acid residues. The amino acid sequence of the component 1 region may have at least 90% sequence identity to SEQ ID NO. 3. The amino acid sequence of the component 1 region may be set forth in SEQ ID NO. 3 or SEQ ID NO. 22. The synthetic peptide may be selected from SEQ ID NOs. 23-36. The synthetic peptide may further comprise an extension of 1-5 amino acid residues at the N terminus. The synthetic peptide may further comprise a lipidation modification. In a particular embodiment, the synthetic peptide is SEQ ID NO. 39. At least one glycine, cysteine, or serine residue of the synthetic peptide may have the lipidation modification. The synthetic peptide may be selected from SEQ ID NOs. 40-44.

Also disclosed herein is a therapeutic composition that comprises insulin or an analog thereof and the synthetic peptide disclosed above. An insulin analog is an altered form of the hormone insulin that is different from any insulin occurring in nature but is still able to perform the same action as human insulin in terms of controlling blood glucose levels. There are two basic types of insulin analogs: fast acting and long acting. Fast acting insulin analogs are more readily absorbed from the injection site and act faster than natural insulin. Examples of fast acting insulin analogs include Lispro, Aspart, and Glulisine. Long acting insulin analogs are released slowly over many hours, supplying the basal level of insulin. Examples of long acting insulin analogs include Detemir insulin, Degludec insulin, and Glargine insulin.

A method of treating insulin resistance in a subject is further described herein. The method comprises administering the synthetic peptide disclosed above to the subject. In some aspects, the subject expresses insulin receptor with site-1 binding-deficient mutations. In some aspects, the subject expresses insulin-desensitized insulin receptor. Thus, also disclosed herein is the use of the synthetic peptide disclosed above for the treatment of insulin and/or the use of the synthetic peptide disclosed above for the manufacture of a medicament to treat insulin resistance. In some implementations, the insulin resistance is caused by site-1 binding-deficient mutations in the insulin receptor or insulin-desensitized insulin receptor.

Also disclosed herein is a method of reducing blood glucose level in a subject. The method comprises administering the synthetic peptide disclosed above to the subject. As shown in the Examples, administration of S597 and its analogs lowered blood glucose levels in subjects. Thus, also disclosed herein is the use of the synthetic peptide disclosed above for the reduction of blood glucose level and/or the use of the synthetic peptide disclosed above for the manufacture of a medicament to reduce blood glucose level. Also disclosed herein is a method of increasing insulin tolerance in a subject. The method may comprise administering the synthetic peptide disclosed above to a subject. Thus, also disclosed herein is the use of the synthetic peptide disclosed above for increasing insulin tolerance and/or the use of the synthetic peptide disclosed above for the manufacture of a medicament to increase insulin tolerance. Also disclosed herein is a method of treating diabetes in a subject. The method may comprise administering the synthetic peptide disclosed above to a subject. The method may also comprise administering insulin or an analog thereof to the subject. Thus, also disclosed herein is the use of the synthetic peptide disclosed above for treating diabetes and/or the use of the synthetic peptide disclosed above for the manufacture of a medicament to treat diabetes. The medicament may comprise insulin or an analog thereof. Also disclosed herein is a method of activating PI3K-AKT pathway without activating MAPK pathway in a cell. The method may comprise administering the synthetic peptide disclosed above to the cell.

As S597 fails to activate IR signaling in cancer cells and thus does not promote cancer cell proliferation, the synthetic peptides disclosed herein are also suitable for use in treating insulin resistance, reducing blood glucose level, increasing insulin tolerance in a subject with cancer. In particular implementations of the above-described methods, the subject is diagnosed with both cancer and diabetes.

### IR Binding and Activation

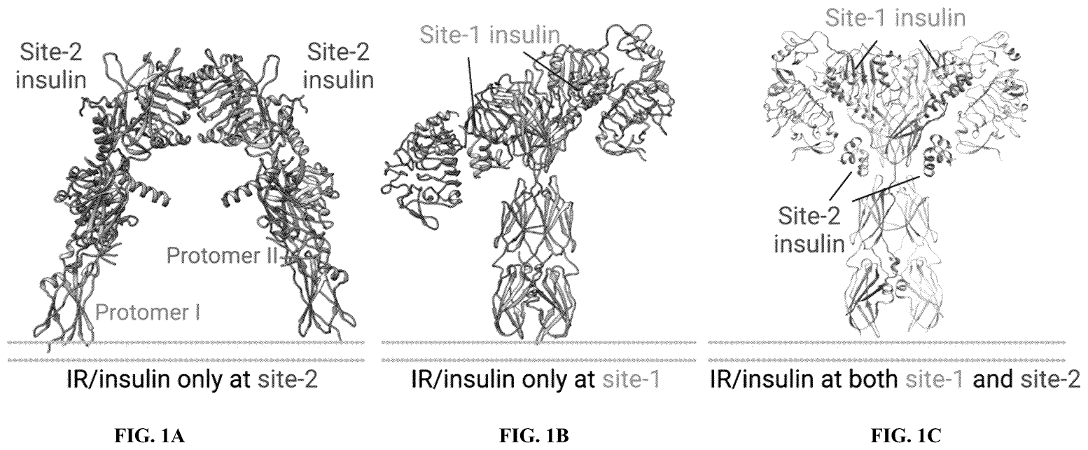

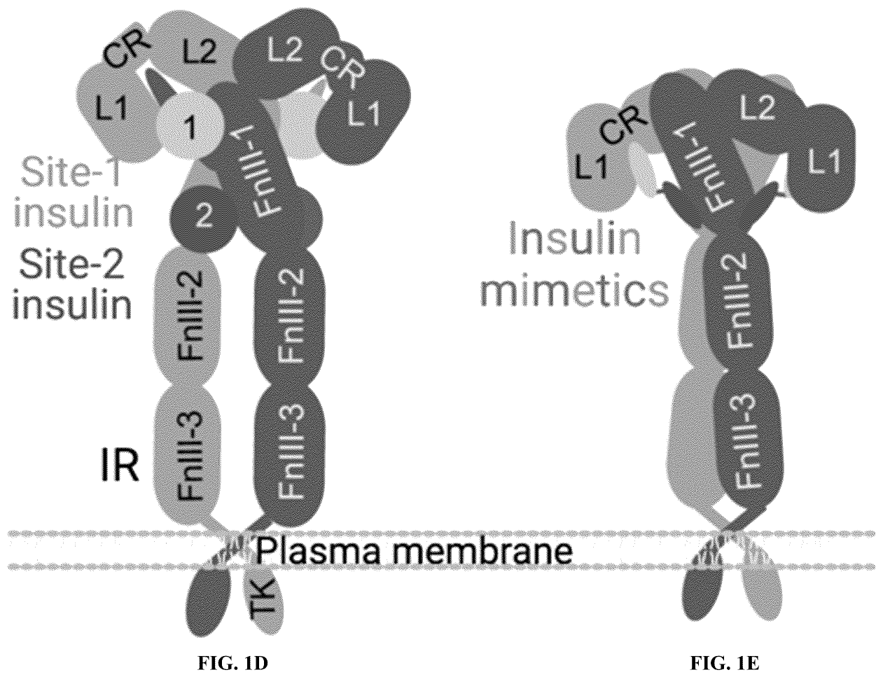

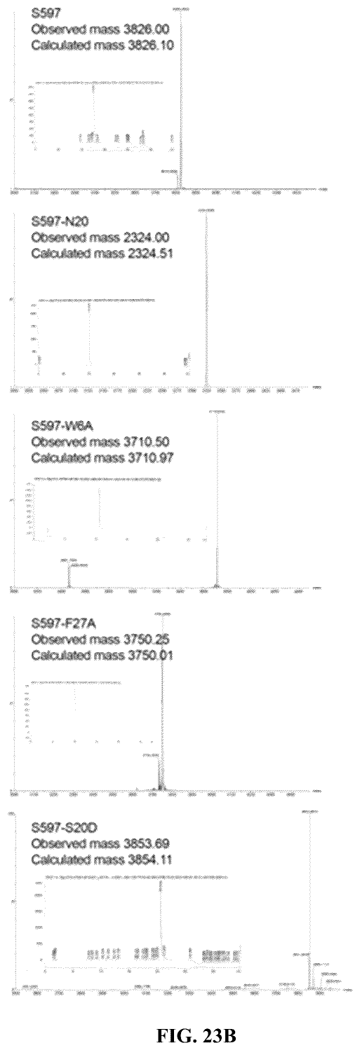

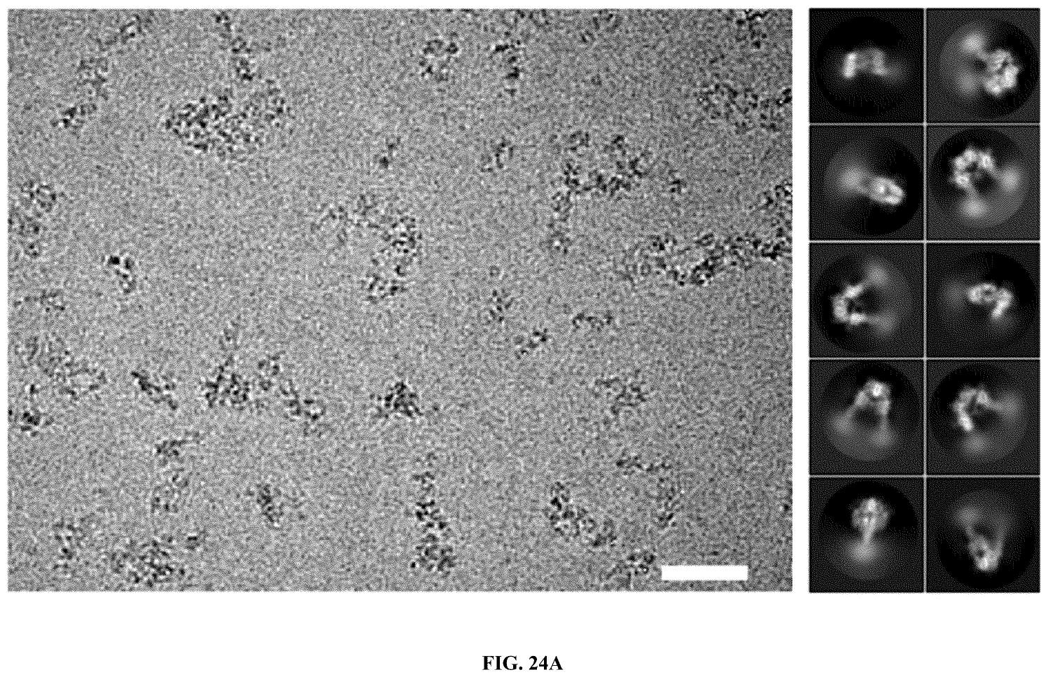

In contrast to other receptor tyrosine kinases, IR is folded and assembled as a stable, disulfide linked dimer in the absence of insulin binding (FIG. 23A). The primary insulin binding site (Site-1) is composed of the L1 domain and the C-terminal region of IRα (α-CT). The secondary insulin binding site (Site-2) is located solely on the FnIII-1 domain. IR forms a stable dimer of multiple disulfide-linked chains IRα and IRB. Each IR protomer consists of leucine rich 1 and 2 (L1 and L2), cysteine rich (CR), three consecutive fibronectin III (FnIII-1, -2, and -3) domains in the extracellular region, and transmembrane (TM) and kinase domains in the intracellular region (FIG. 1). The C-terminal region (αCT) of the IRα chain is always associated with L1 domain and essential for insulin binding. The two membrane-proximal FnIII-3 domains in the insulin free IR (apo-IR) are separated by ˜120 Å and form a ‘Λ’-shaped conformation. Insulin binding disrupts the apo-state of IR and triggers a large conformational change of IR from ‘Λ’- to ‘T’-shape. As a result, the distance between two FnIII-3 domains connected to TM and kinase domains in the insulin-bound ‘T’-shape IR dimer is reduced (˜40 Å), enabling IR kinase activation.

The cryogenic electron microscopy (cryo-EM) structure of insulin-bound IR at 3 Å resolution shows that IR activation requires the binding of 4 insulins per IR dimer, instead of 2 insulins as previously thought (FIG. 1C). Because of the 2-fold symmetry, there are 2 distinct types of insulin binding sites, termed site-1 and site-2 above. The primary insulin binding site (site-1) comprises several structural elements from both protomers, including L1/αCT of one protomer and a FnIII-1 loop of the other. Insulin at IR site-1 crosslinks two IR protomers, thus stabilizing the ‘T’-shaped active IR dimer. Site-2 exists in one side of a major β sheet of the FnIII-1 domain of IR (FIGS. 1A and 1C). The binding of multiple insulin monomers to both site-1 and site-2 synergistically induces complex conformational changes of IR from the Λ-shaped apo-IR dimer to a T-shaped symmetric IR dimer with full activity. Inhibition of insulin binding to IR site-2 leads to an asymmetric conformation and only partial activation of IR. Furthermore, insulin binding to IR site-1 leads to asymmetric conformation of IR and partial activation (FIG. 1B). The insulin binding to IR site-2 breaks the asymmetric IR dimer, thus promoting a more stable, symmetric ‘T’-shape IR dimer. This discovery establishes a truly unique and comprehensive molecular mechanism of IR activation and provides insights into the design of insulin mimetics.

Phage display affinity screening identified that synthetic peptide S597 (SEQ ID NO. 23) can bind to the IR with sub-micromolar affinity while lacking any sequence homology to insulin. S597 induces robust IR auto-phosphorylation and potently activate IR signaling. In particular, SS597 lowers blood glucose and increases de novo lipogenesis in liver and deposition of triglycerides in adipose tissue of Zucker diabetic fatty rats, suggesting that S597 could activate insulin-like signaling in vivo. Furthermore, S597 protects against atherosclerosis associated with metabolic syndromes by reducing leukocytosis. Notably, S597 activates the AKT pathways as effectively as insulin but is less efficient in activating the MAPK pathways in blood leukocytes and skeletal muscles. There is a long-standing notion that “metabolic” pathways from IR are mediated by PI3K-AKT, while “mitotic” pathways are mediated by MAPK. However, PI3K-AKT pathways activate mTOR complex to regulate cellular metabolism and growth, the PI3K-AKT pathway is close to the most mutationally activated signaling pathway in cancer, and crosstalk between two pathways is evident. This biased signaling by S597 suggests that the insulin mimetic engages the IR differently from native insulin, inducing a distinctive and unique conformation of the IR during receptor activation. The observation that this peptide agonist selectively activates the IR signaling in different tissues suggests a possible avenue by which structural differences of the IR in complex with designed analogs could regulate IR signaling in unique ways for the enhancement of diabetes and severe insulin resistance treatment and open a new area of future research.

S597 has two structural components that simultaneously bind two distinct regions on the IR, which are referred to herein as component 1 region (also referred to herein in as “S597-component-1” and in some aspects comprises the sequence set forth in SEQ ID NO. 3) and component 2 region (also referred to herein in as “S597-component-2” and in some aspects comprises the sequence set forth in SEQ ID NO. 1). As shown in the Examples, the structure of the L1 domain of IR in complex with the S597-component-1 alone has been determined by X-ray crystallography and illustrates how the S597-component-1 engages the IR.

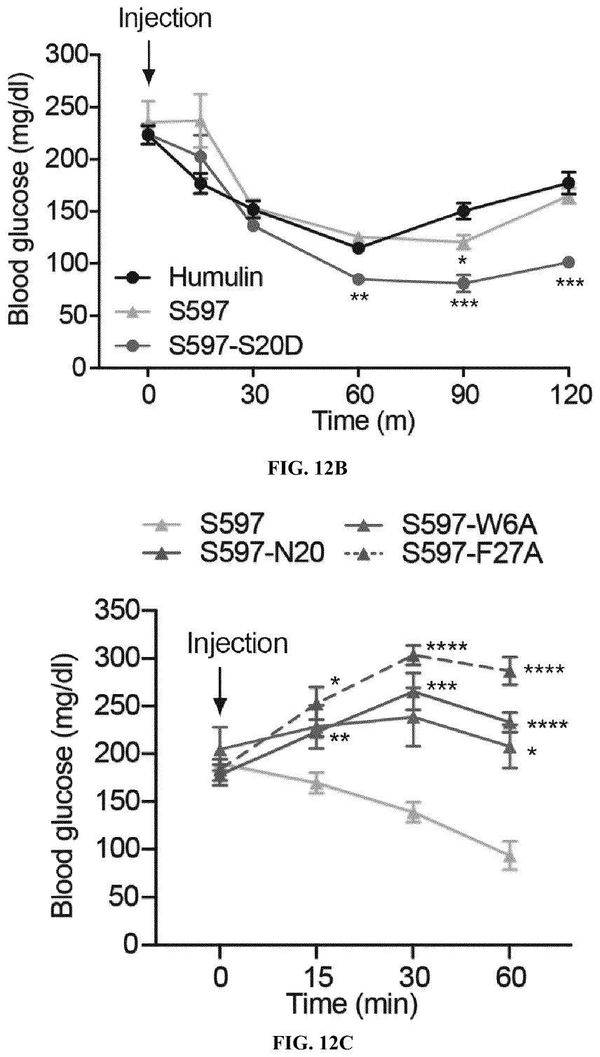

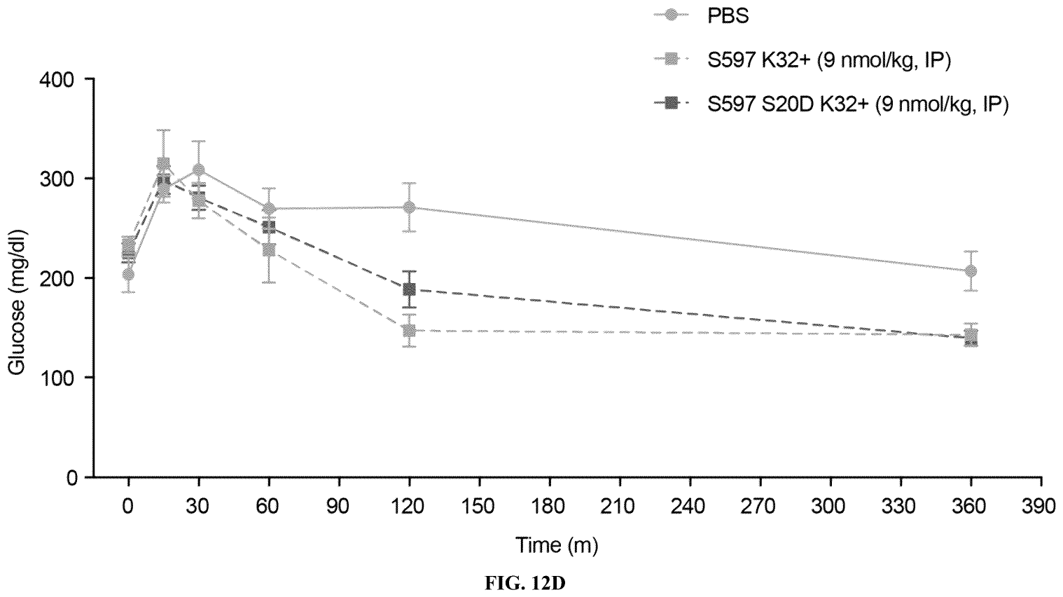

Knowing the structure of IR/S597 complex enabled the design of separation-of-function mutants that reduce the interaction between S597 and IR. Mutants of S597 with site specific mutations in either the component 1 or the component 2 region have greatly reduced potency in inducing IR activation (FIG. 12A). The cryo-EM structure of full-length IR bound with the S597-component-2 alone at a resolution of 3.5 Å shows that S597-component-2 is folded as a short α-helix and binds to the side-surface of the FnIII-1 domain of IR. Similarly, the cryo-EM structure of the IR in complex with the full-length S597 at a resolution of 5.4 Å shows that, unlike insulin, which binds to two distinct sites on the IR at a maximal 4:1 stoichiometry (insulin: IR dimer), in each half of the IR/S597 complex, one S597 concurrently binds to both the L1 domain of one protomer and the FnIII-1 domain of another, resulting in a 2:1 stoichiometry (S597: IR dimer) (FIGS. 1E, 6A, and 12A). The binding of two S597 molecules to the IR induces and stabilizes an extended T-shaped conformation that allows the intracellular kinases to undergo efficient auto-phosphorylation. These data demonstrate that both S597-component-1 and S597-component-2 are required for IR activation and reveal how insulin mimetics activate IR kinase.

Importantly, S597 fully activates native IR and IR mutants that disrupt insulin binding or destabilize the insulin-activated IR, thereby eliciting insulin-like signaling. S597 is not expected to activate mutants of the IR that may cause misfolding or defects in trafficking or kinase activity. Because S597 binds and activates IR in a unique manner, S597 fully activates IR mutants that disrupt insulin binding or destabilize the insulin-induced compact T-shape. The elucidation of S597-induced activation mechanism for the IR indicates that insulin mimetics can restore normal IR signaling in severe insulin resistance diseases. More importantly, these data indicate that such peptide mimetics are useful for treating these fatal congenital diseases. As S597 and S597 analogs provide a significant advantage over a previously described insulin mimetic that showed low receptor specificity, insulin in combination with S597 could lower insulin requirements in patients with diabetes. Such insulin mimetics may restore normal glucose and lipid metabolism in severe insulin resistance syndromes.

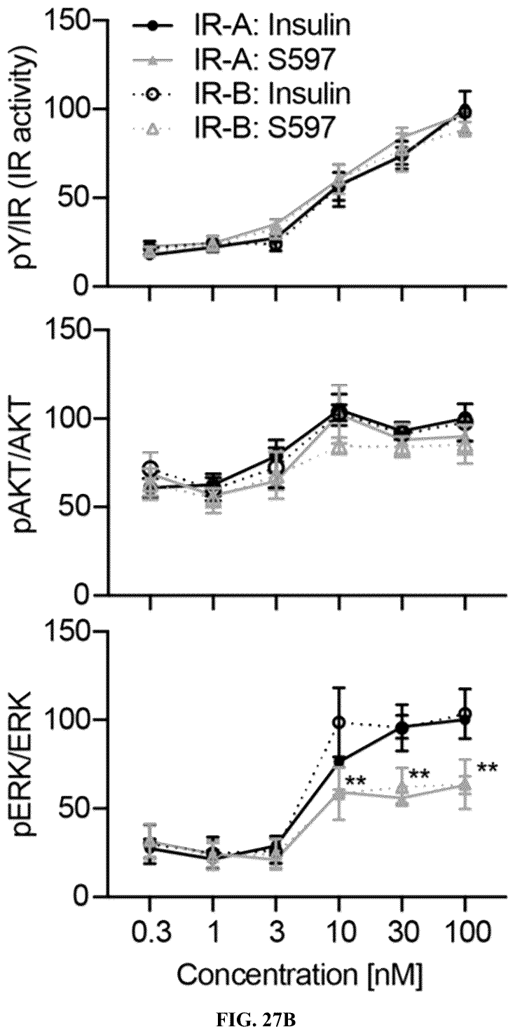

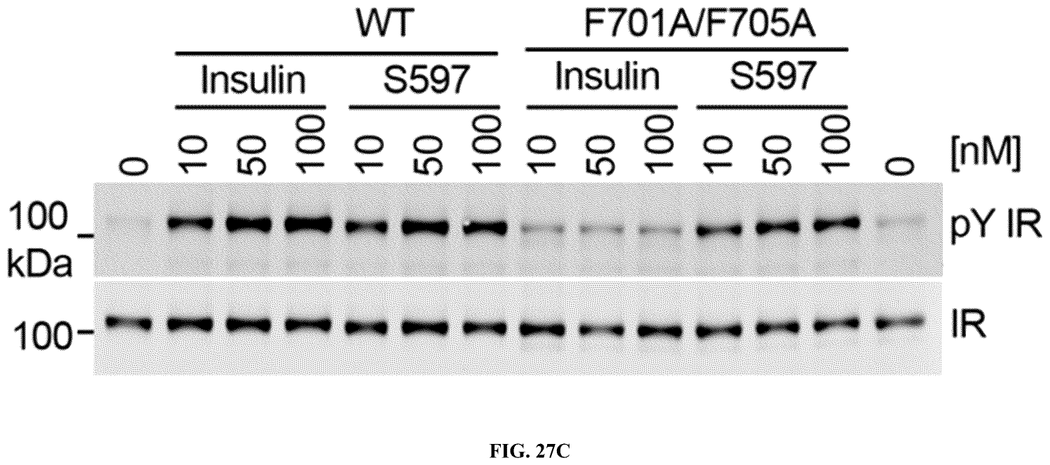

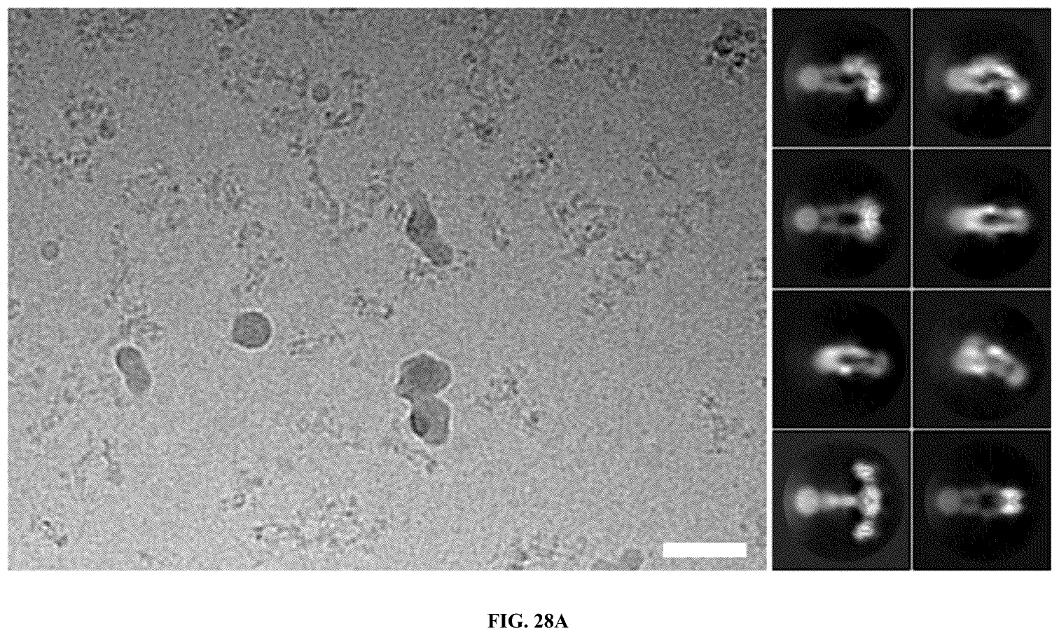

It should also be noted that S597-induced IR activation activates AKT pathways as effectively as insulin but is less effective in activating MAPK pathways. In liver, S597 activates both the PI3K-AKT and MAPK pathways, and induces IR endocytosis, which is similar to insulin. Liver is the major organ for insulin clearance mediated by IR endocytosis, and defects of this process can cause hyperinsulinemia. The fact that S597 induces IR internalization suggests that S597 is cleared and elicits insulin-like signaling in the liver. Interestingly, in skeletal muscle, S597 selectively activates the PI3K-AKT pathways, while only weakly activating the MAPK pathway. S597 significantly weakens mitogenic signaling in monocytes and hematopoietic stem cells, and reduces local inflammation, thereby protecting atherosclerosis associated with metabolic syndrome in mice. The fact that the biased signaling is observed in skeletal muscle but not in liver suggests that the relative expression of IR, IGF1R, and IR/IGF1R heterodimer contribute to the selectivity of IR signaling, as S597 specifically binds and activates IR, but not IGF1R. Indeed, healthy hepatocytes or mature adipocytes do not express appreciable levels of IGF1R and predominantly express IR, whereas skeletal muscle cells express both IR and IGF1R. Furthermore, although S597 did not change the IR auto-phosphorylation in cells overexpressing either IR-A or IR-B (FIGS. 27A-27B), contributions from heterodimers formed between IR-A and IR-B (i.e., IR-A/IR-B), or even between IR isoforms and IGF1R (i.e., IR-A/IGF1R or IR-B/IGF1R) in vivo, cannot be ruled out.

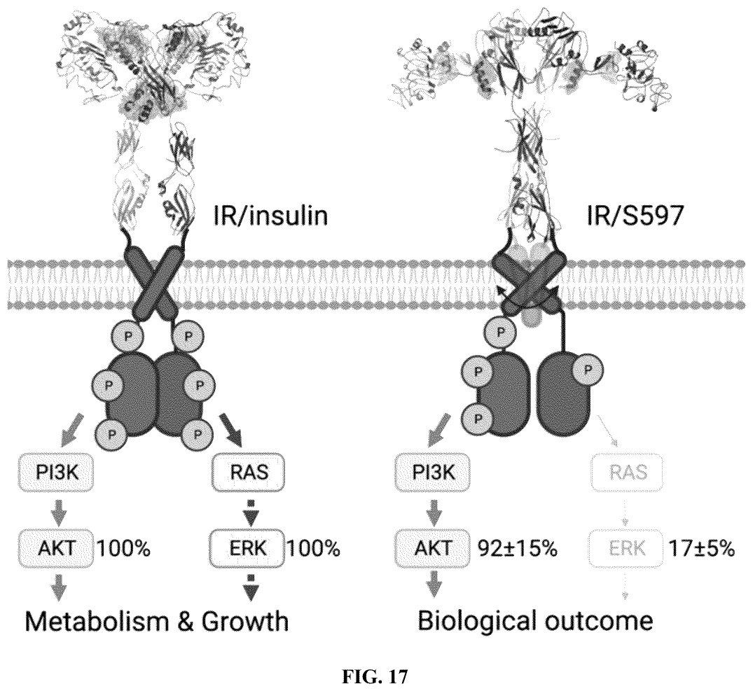

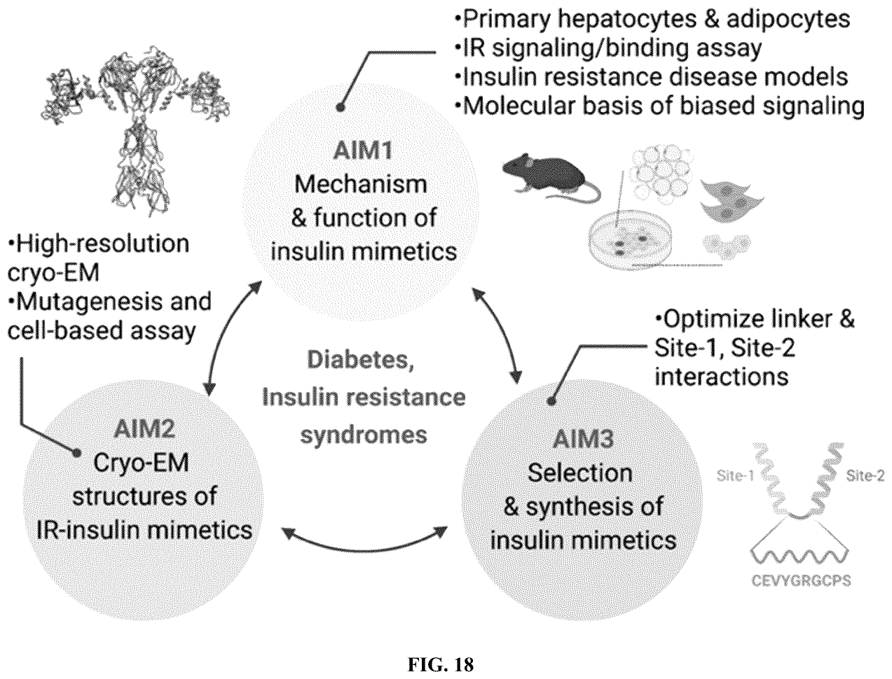

Signaling selectivity of S597 agonist action can be modulated by modest sequence changes, such as in the exemplary variations of S597 represented in SEQ ID NOs. 23-46. Mutants of S597 with site specific mutations in either the component 1 or the component 2 region greatly reduced potency in inducing IR activation. The preliminary cryo-EM structural studies showed that the TM domains of IR/S597 analogs adopt different conformations. This suggests that S597 generates distinct biased signaling through allosterically modulating the conformation of TM domains of IR during activation (FIG. 17). The structural models described herein may inform the design of new S597-like peptides with stronger receptor binding affinity and higher structural stability. Such peptides may recapitulate insulin-like signaling and hold promise for the treatment of insulin-resistant conditions (FIG. 18). As such, the present disclosure could open a new area of future research and enhance the treatment of diabetes (e.g., biased agonist that activate only the metabolic branch of IR signaling) and severe insulin resistance (e.g., insulin-like agonist that recapitulate true insulin-like signaling).

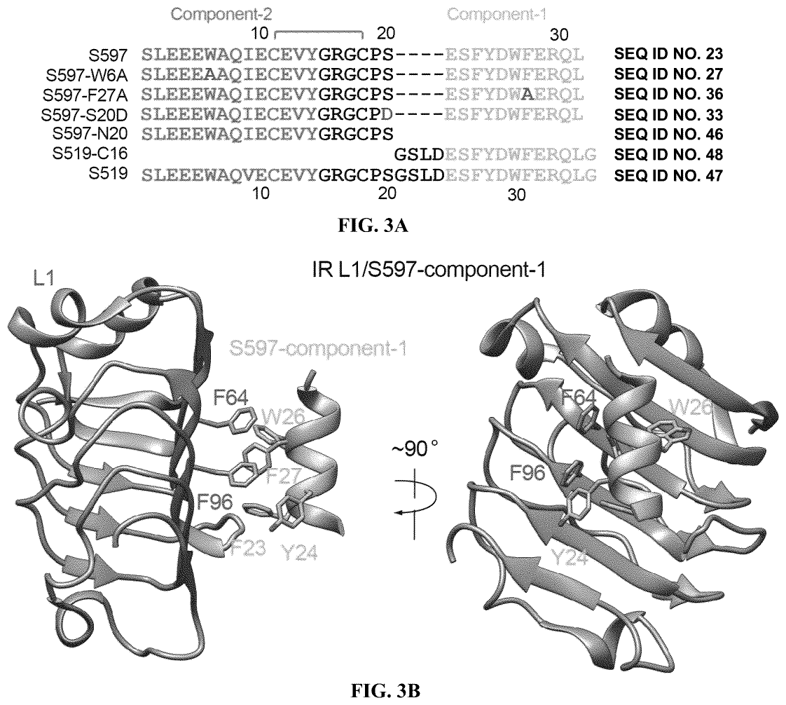

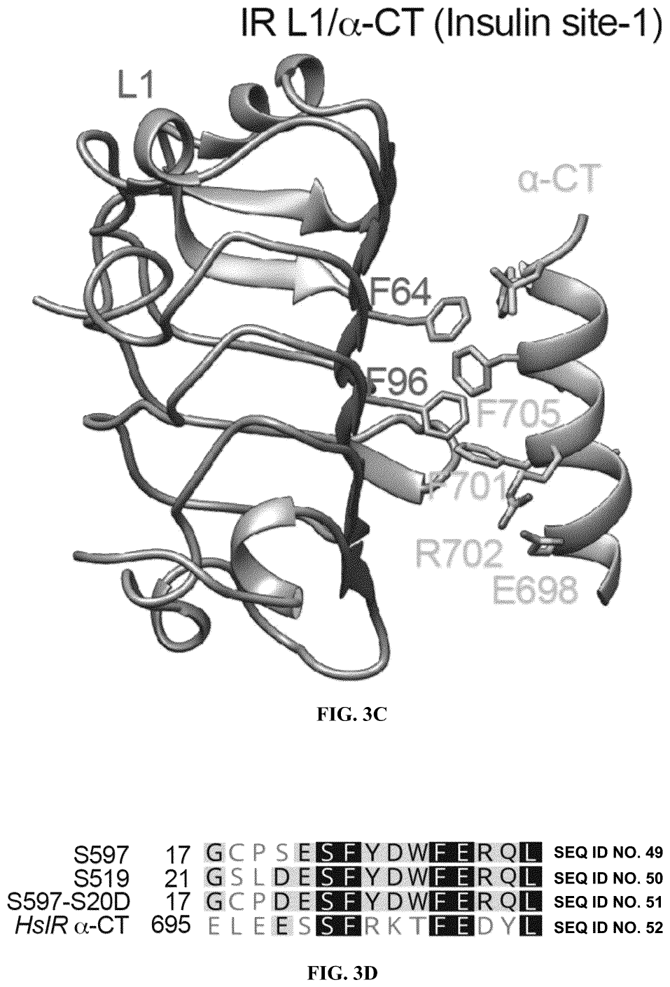

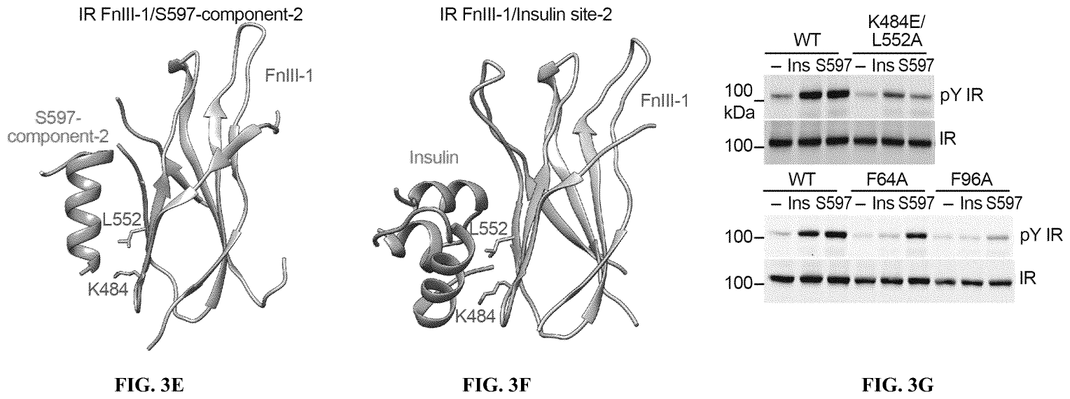

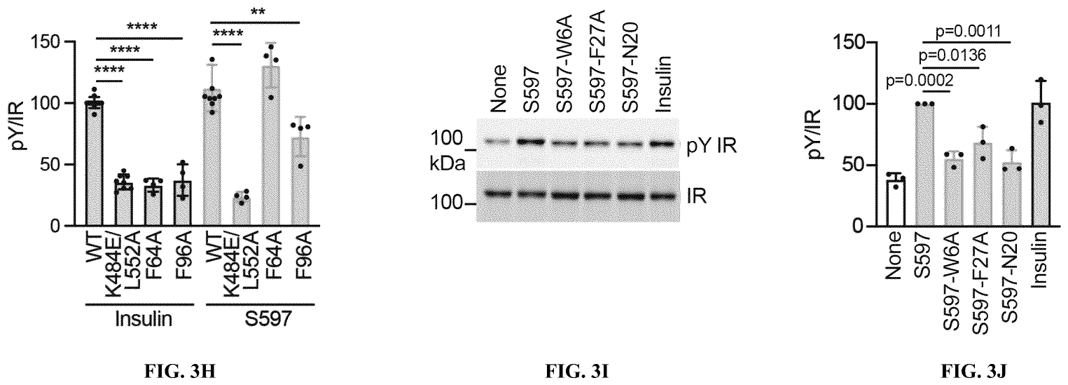

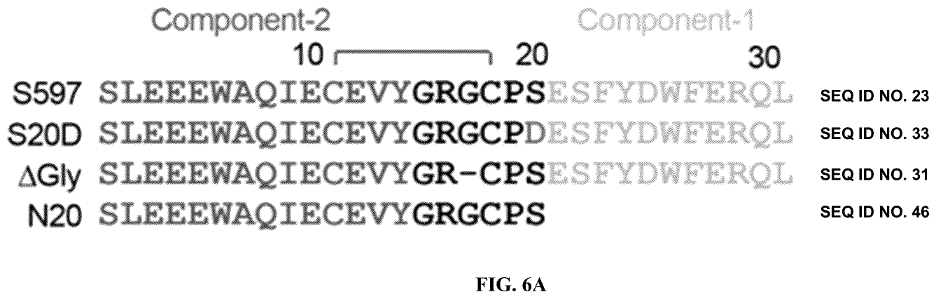

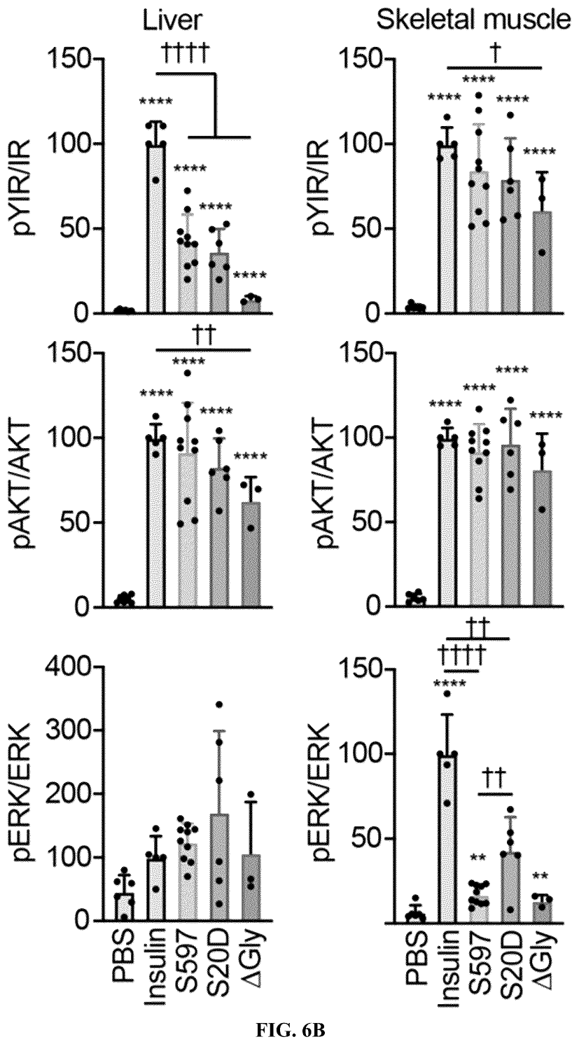

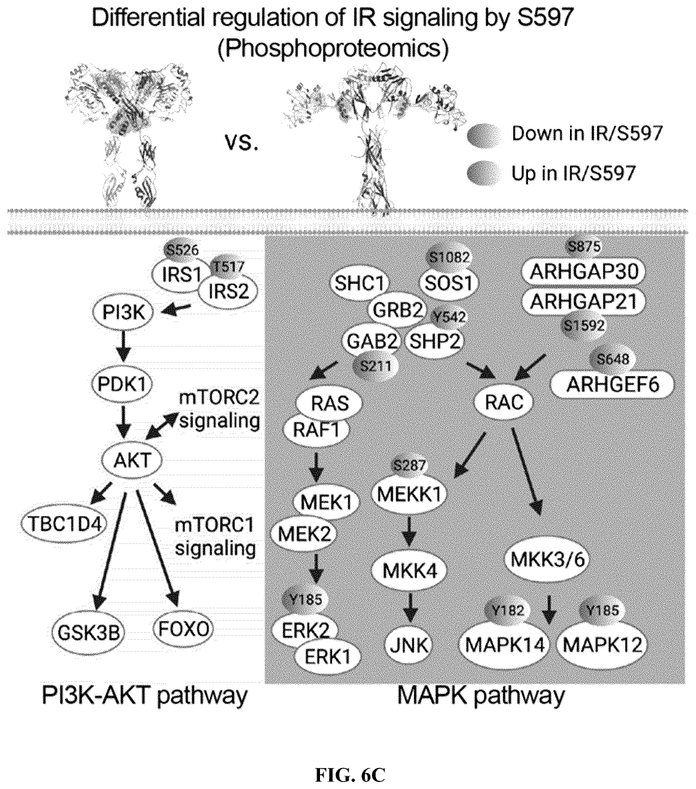

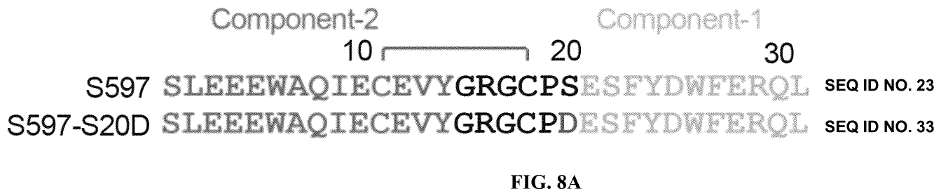

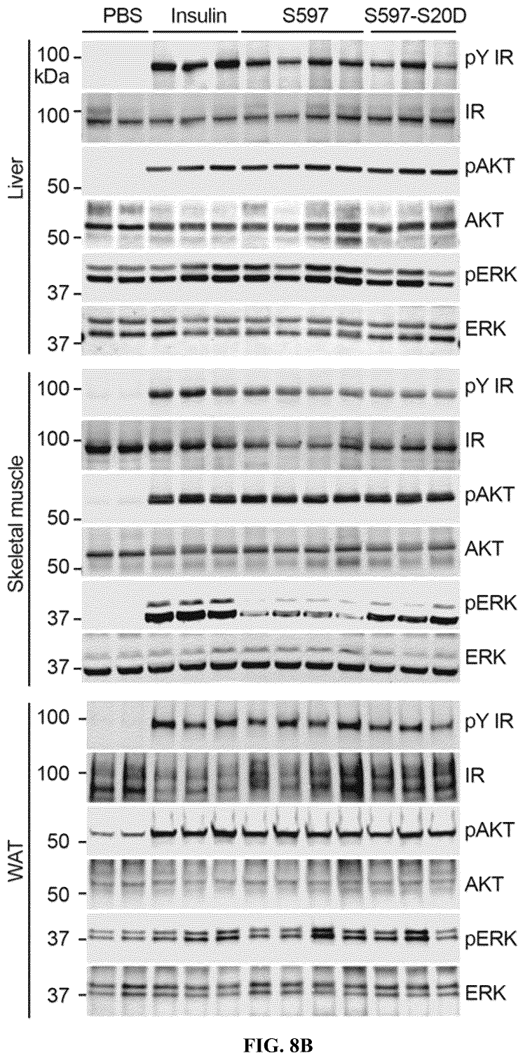

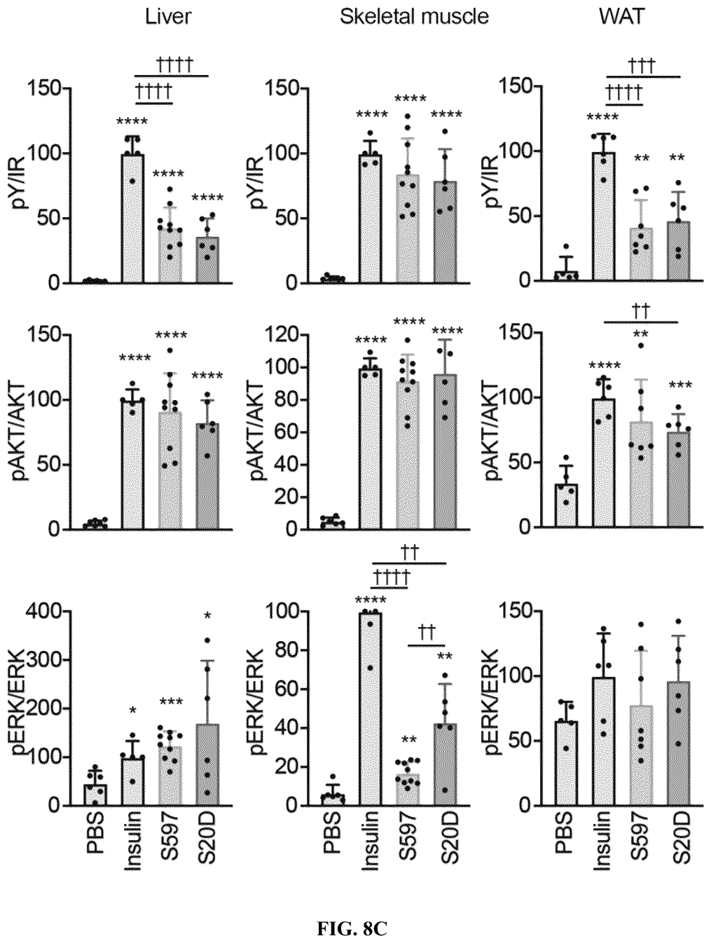

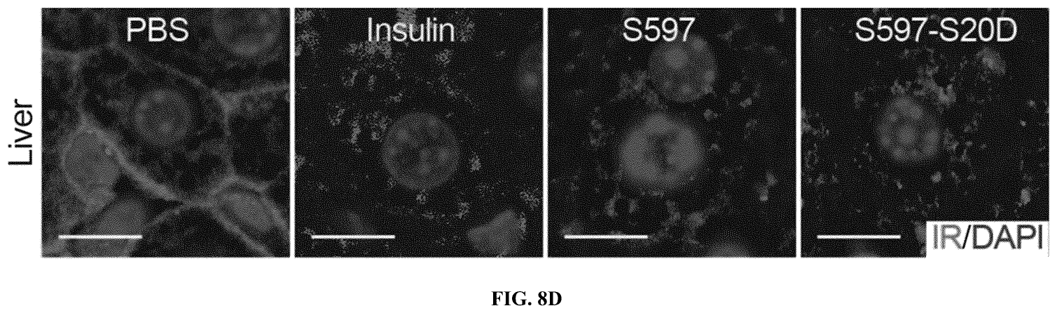

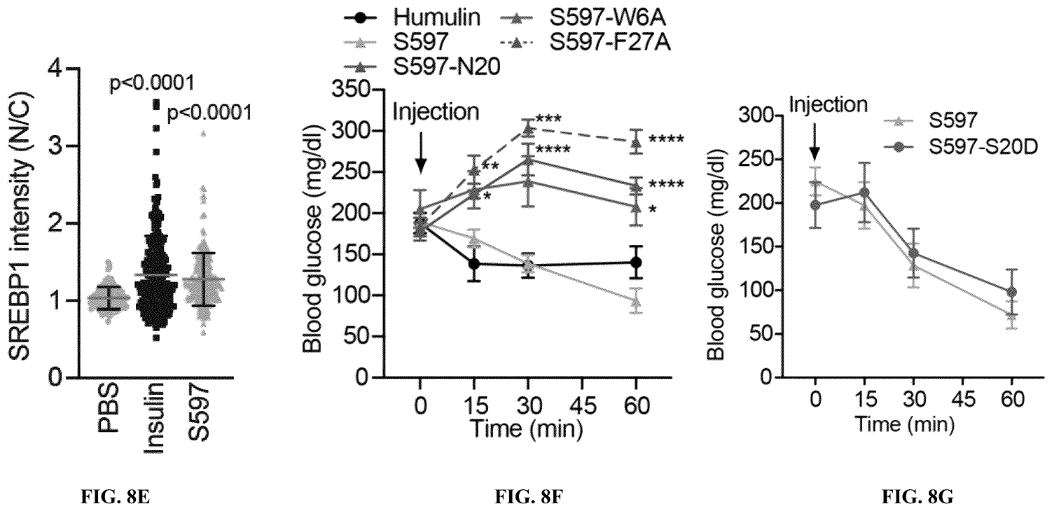

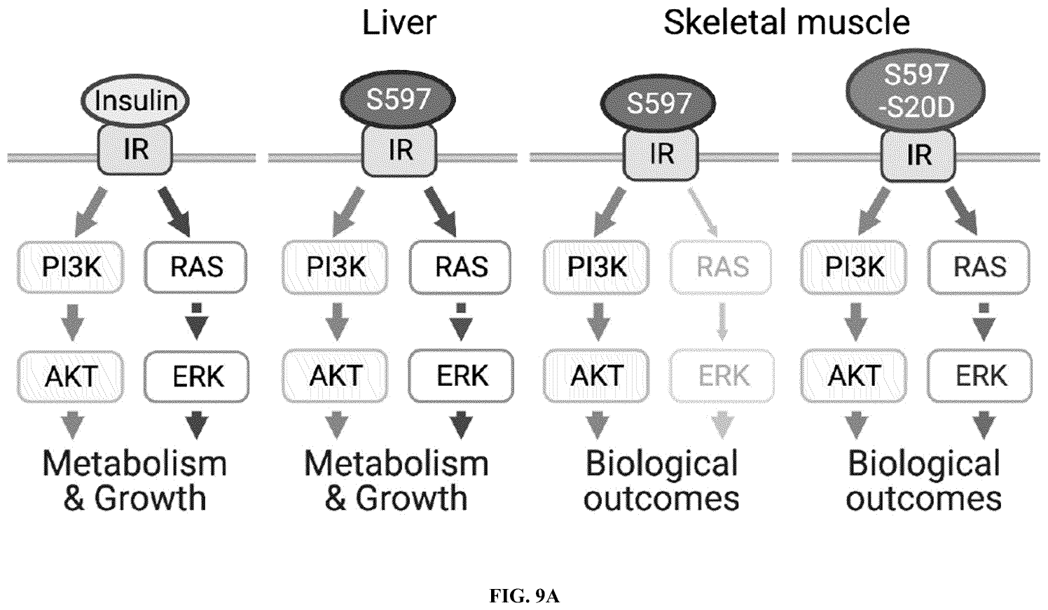

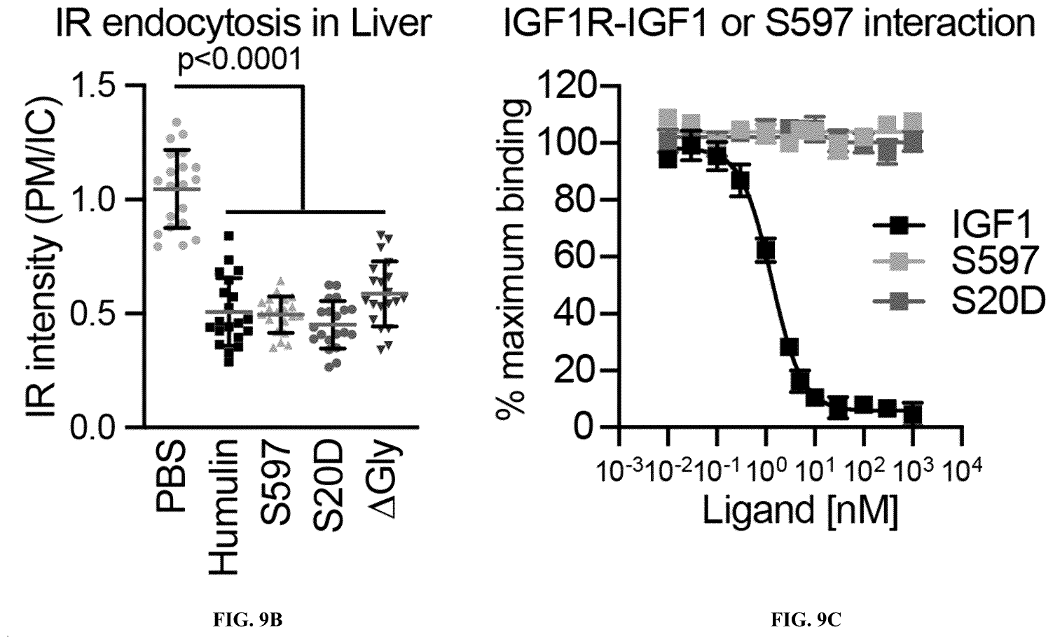

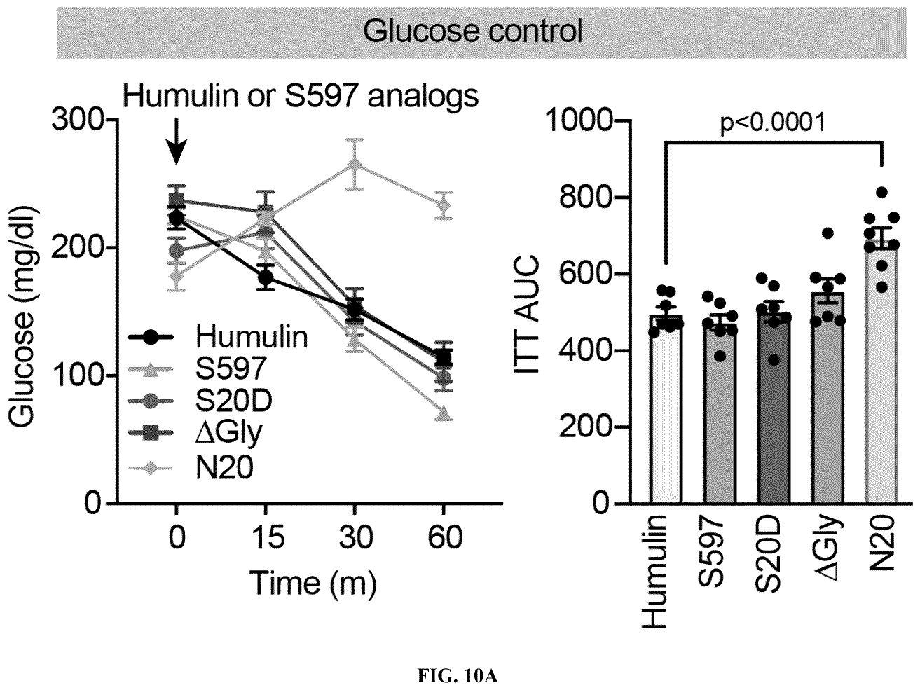

S597-component-1 and IR α-CT share high sequence similarity and bind the L1 domain of IR in a similar manner (FIG. 3D). In the structure of IR L1/α-CT, the salt bridge formed between Glu698 in the α-CT and Arg188 in the L1 domain maintains the α-helical conformation of the IR α-CT. Such interaction does not exist in the structure of IR L1/S597-component-1, as the residue corresponding to Glu698 of IR α-CT is a serine in S597 (Ser20 S597) (FIG. 8A). Consequently, the S597-component-1 adopts a shorter α-helix with relative to IR α-CT (FIGS. 3B, 3C). Thus, substitution of the S597 serine at 20 to an aspartic or glutamic acid could form a salt bridge with the IR L1 domain, stabilizing the IR L1/S597-component-1 interaction and thereby modulating the agonist action of S597. Like S597, S597-S20D (SEQ ID NO. 33) increased the levels of pY IR and pAKT in the liver and skeletal muscle of mice (FIGS. 6B, 8B-8C, 9A) and promoted IR endocytosis in the liver (FIGS. 8D, 9B). However, activation of IR by S597 and the S597-S20D lead to distinct biased downstream signaling. The PERK levels in both the liver and skeletal muscle of mice treated with S597-S20D were significantly increased, in contrast to S597, where only the pERK levels in the liver but not in the skeletal muscle were increased (FIGS. 6B, 8B, 8C, 9A). To explore the possibility of cross talk between S597-S20D and IGF1R, the IGF1R-S597-S20D interaction was analyzed. In vitro competition assay showed that both S597 and S597-S20D do not bind IGF1R, arguing against the idea that S597-S20D interacts more with IGF1R (FIG. 9C).

S597-ΔGly (deletion of S597 Gly at 17) is another S597 analog expected to reduce the flexibility of S597. As shown in FIG. 6B, S597-ΔGly less potently activated IR signaling in both liver and skeletal muscle, which suggests that the agonist action of S597 can be modulated by a very modest sequence change.

### Examples

### Example 1. Activation of the insulin receptor by an insulin mimetic peptide

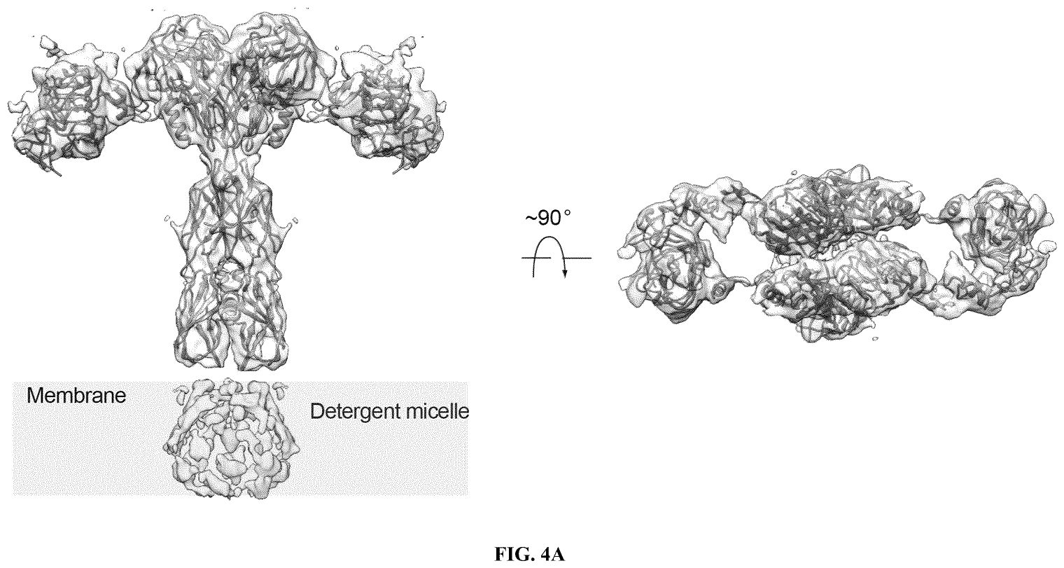

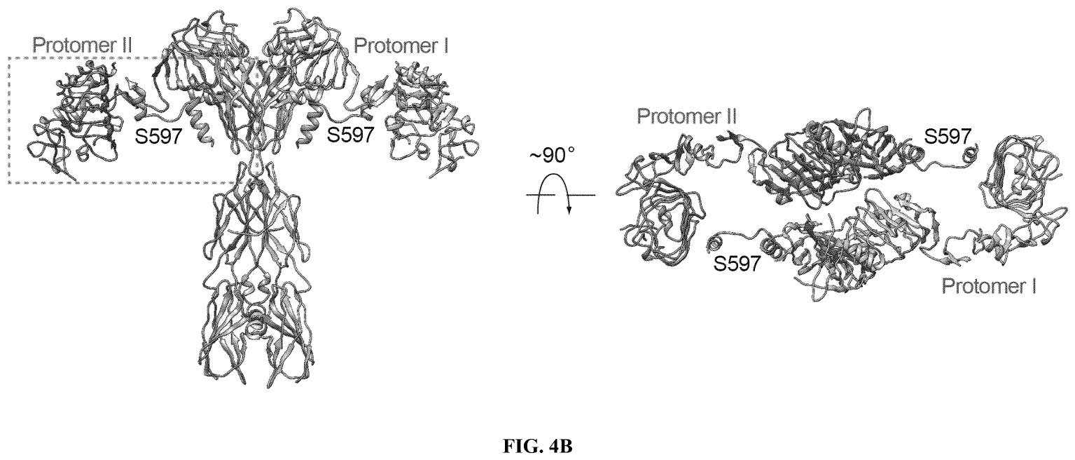

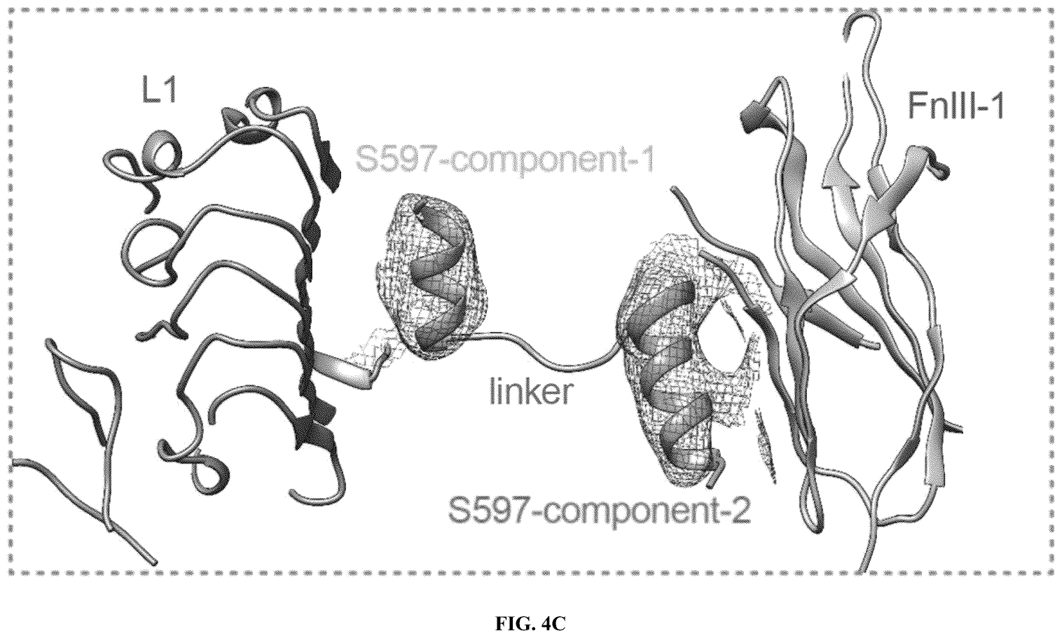

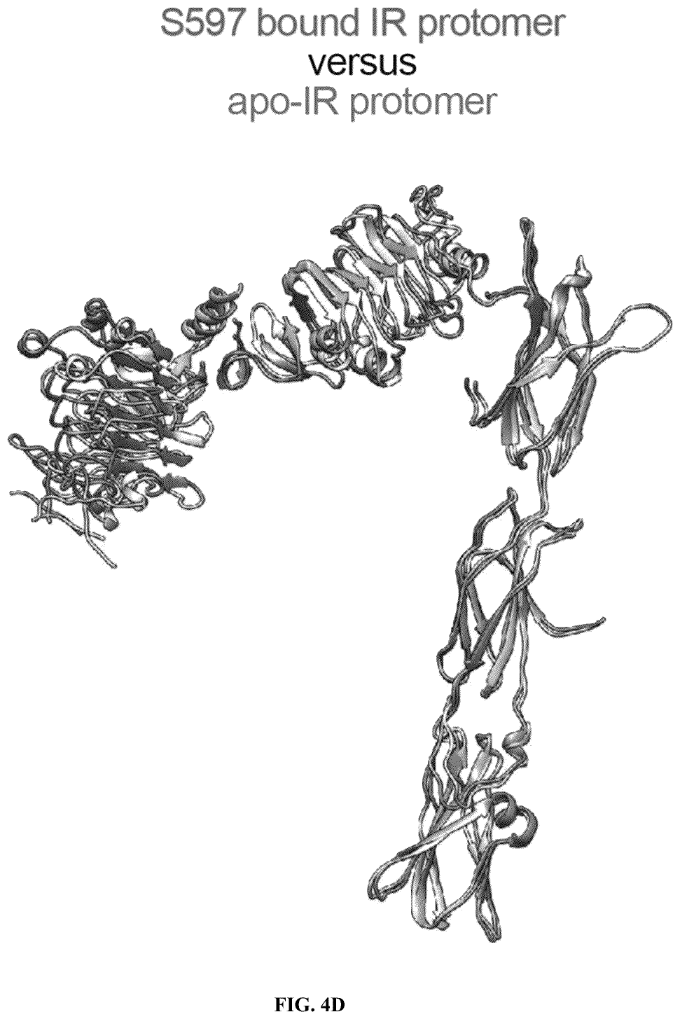

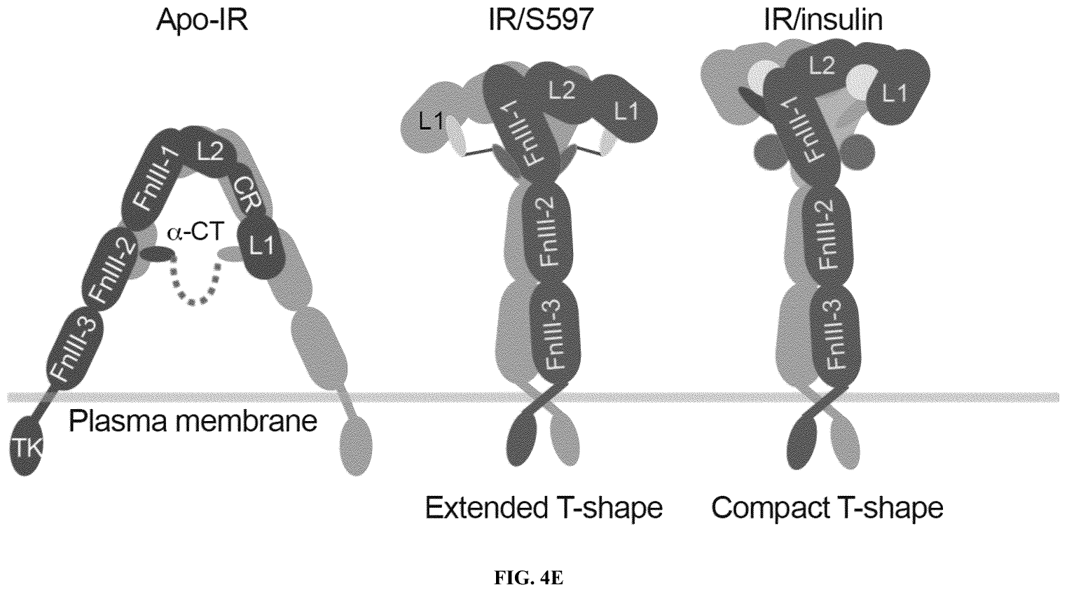

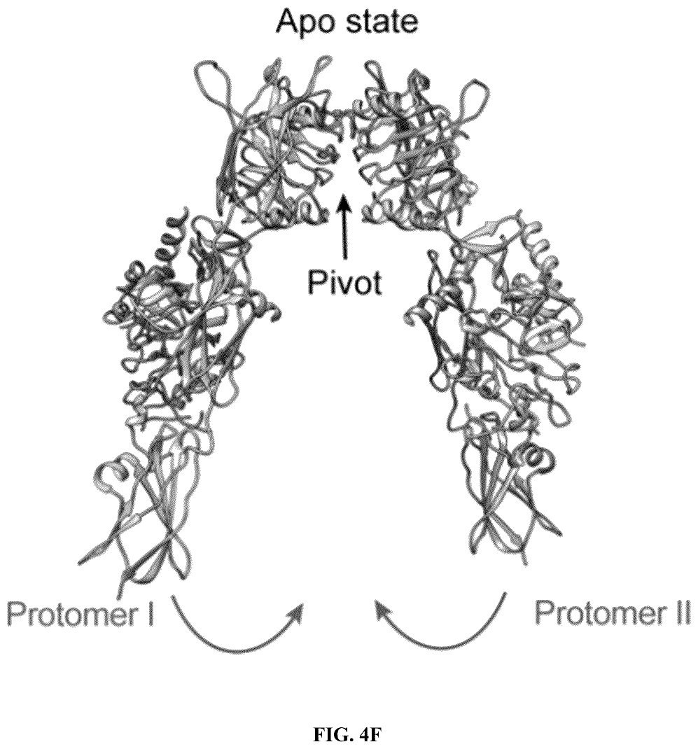

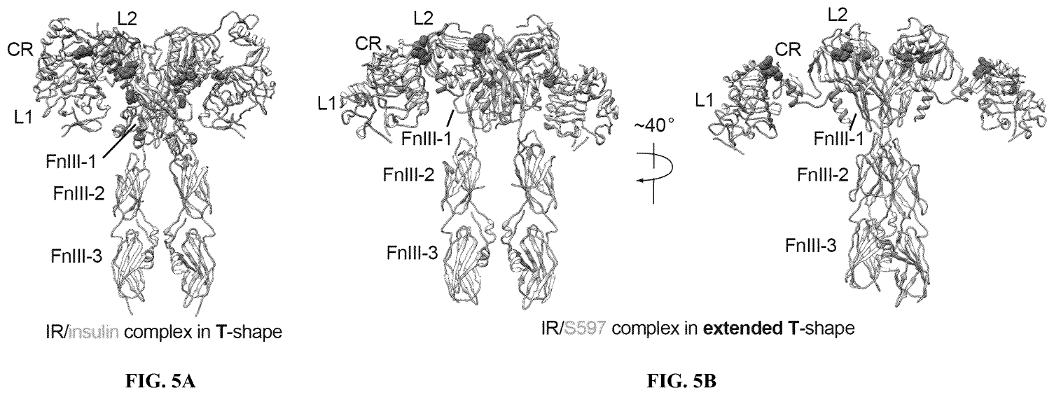

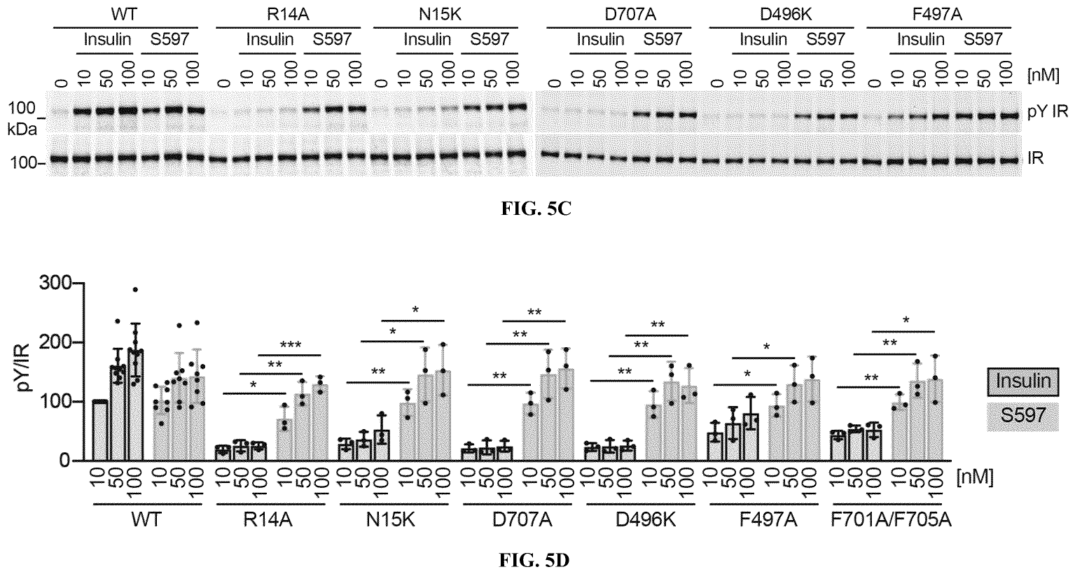

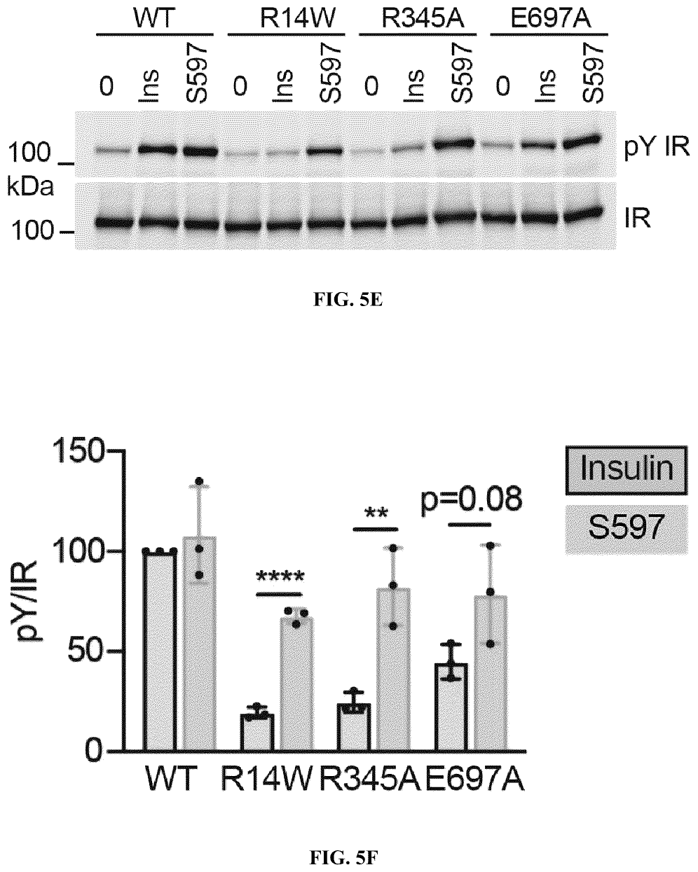

The binding of multiple insulins to the IR disrupts the auto-inhibited state of IR, inducing a large conformational change between the two protomers, and ultimately promoting a compact T-shaped active conformation, allowing the intracellular kinase domains to undergo efficient auto-phosphorylation. S597 can activate IR as efficiently as insulin but via an alternative mechanism. The binding of S597 to apo-IR first delocalizes the α-CT motif from the L1 domain by competing for the same binding surface on the L1 domain. The delocalization of the α-CT motif then leads to the destabilization of the A-shaped auto-inhibited conformation of IR. Indeed, the α-CT motif was not observed in the IR/S597 complex, which suggests that once the α-CT is released from the L1 domain, it becomes disordered. Subsequently, the two protomers would undergo rigid-body rotation using the L2-FnIII-1 interface as the hinge. Finally, the active state of IR is stabilized by the concurrent binding of S597 to the L1 domain of one protomer and the FnIII-1 domain of another. In this binding mode, the L1 domain of one protomer is oriented laterally to the FnIII-1 domain of the adjacent protomer, resulting in an extended-T shaped symmetric conformation (FIGS. 4A-4F). Notably, the IR/S597 complex is less rigid than IR/insulin complex, due to the flexible linker between the two components of S597. Nevertheless, the distance and orientation between two membrane-proximal domains are almost identical between IR/S597 and IR/insulin complexes, explaining why S597 can activate IR similarly to insulin (FIGS. 5A, 5B).

The cryo-EM structure of full-length IR bound with the S597-component-2 alone was determined at a resolution of 3.5 Å, showing that S597-component-2 is folded as a short α-helix and binds to the side-surface of the FnIII-1 domain of IR. The cryo-EM structure of the IR in complex with the full-length S597 was solved at a resolution of 5.4 Å. Unlike insulin, which binds to two distinct sites on the IR at a maximal 4:1 stoichiometry (insulin: IR dimer), in each half of the IR/S597 complex, one S597 concurrently binds to both the L1 domain of one protomer and the FnIII-1 domain of another, resulting a 2:1 stoichiometry (S597: IR dimer). The binding of two S597 molecules to the IR induces and stabilizes an extended T-shaped conformation that allows the intracellular kinases to undergo efficient auto-phosphorylation. This ‘extended T’-shaped IR is stabilized by the concurrent binding of S597 to both L1 domain of one IR protomer and FnIII-1 of the other. Strikingly, the structural and functional results demonstrate that, because S597 binds and activates IR in a unique manner, S597 fully activates IR mutants that disrupt insulin binding or destabilize the insulin-induced compact T-shape. Collectively, this work elucidates the S597-induced activation mechanism for the IR and suggests that insulin mimetics can restore normal IR signaling in severe insulin resistance diseases. More importantly, these data imply that such peptide mimetics may be viable therapeutics for treating these fatal congenital diseases.

a. The Structure of Full-Length IR Bound with the S597-Component-2

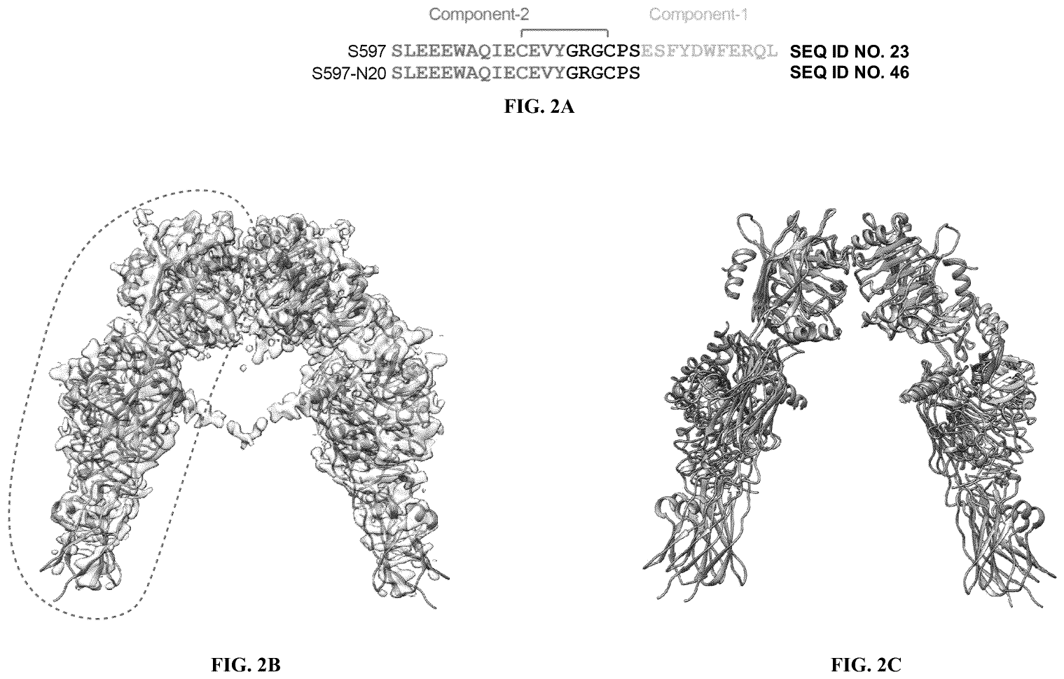

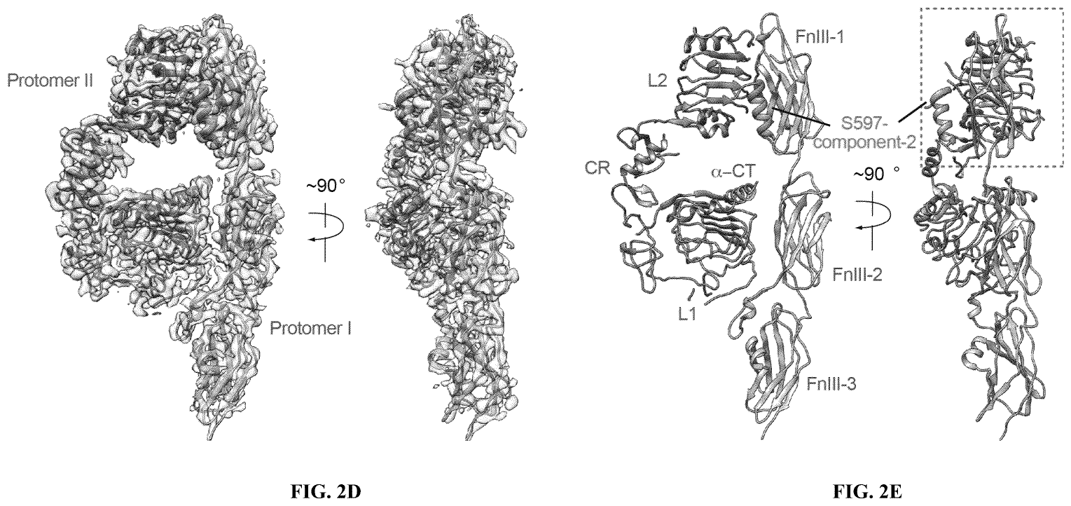

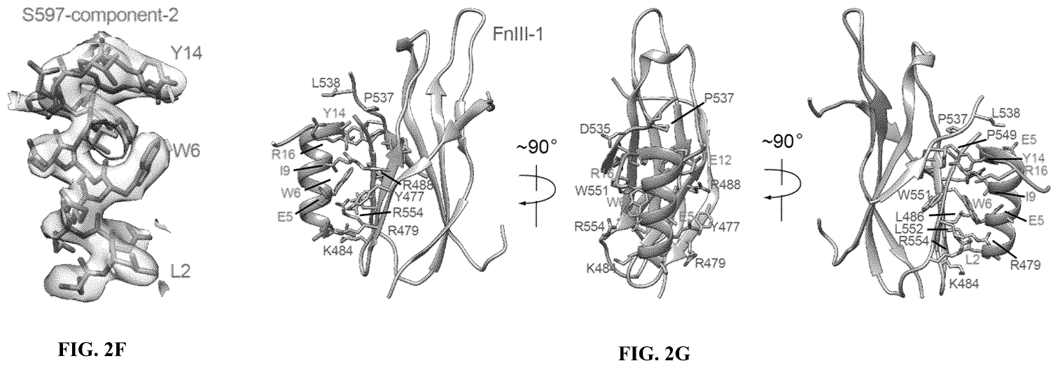

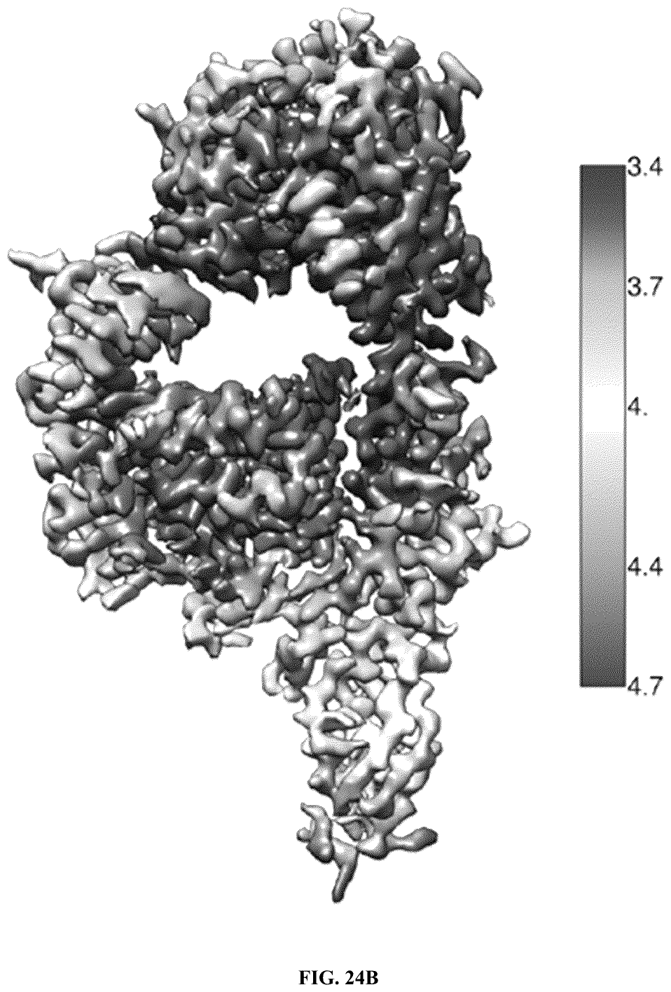

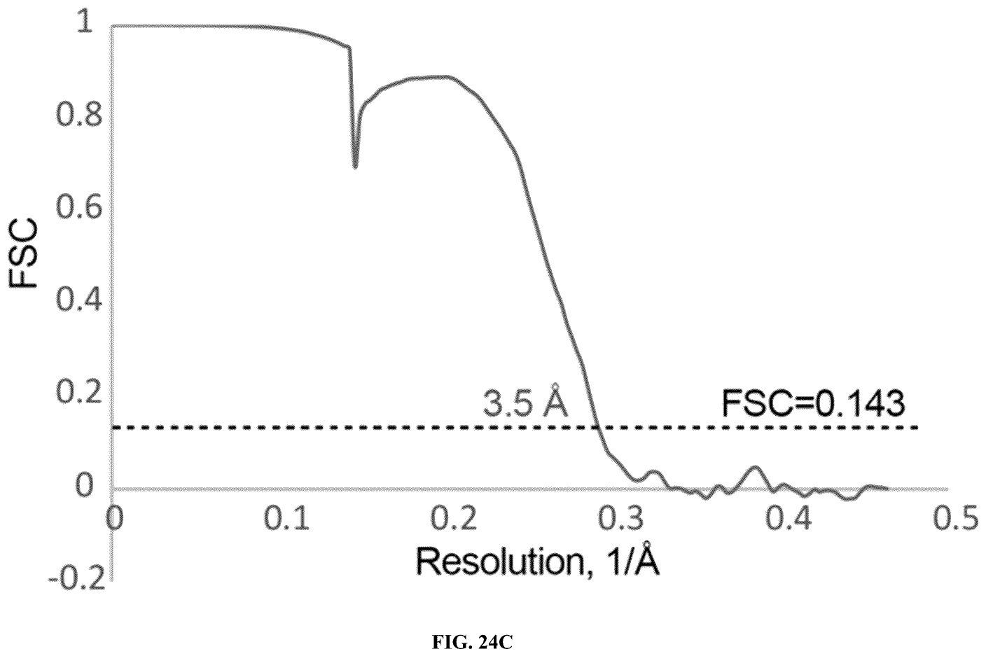

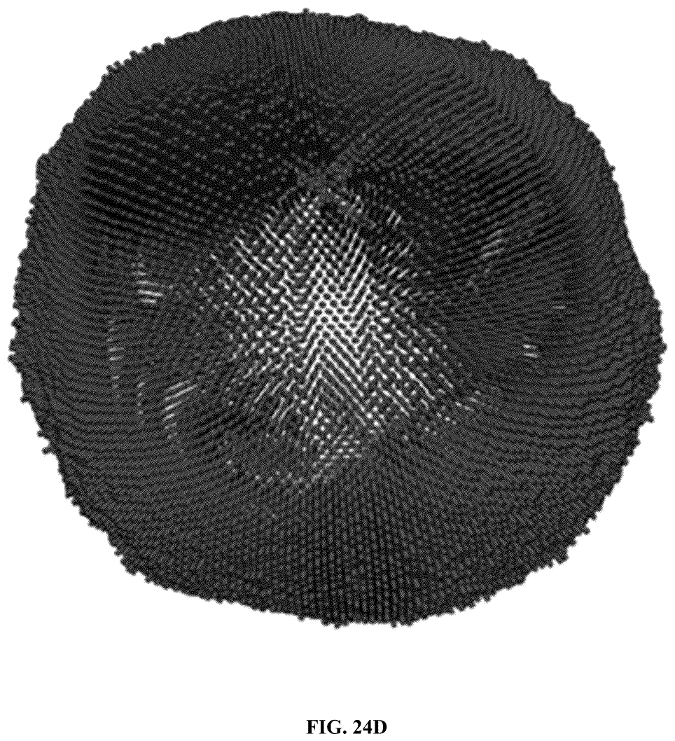

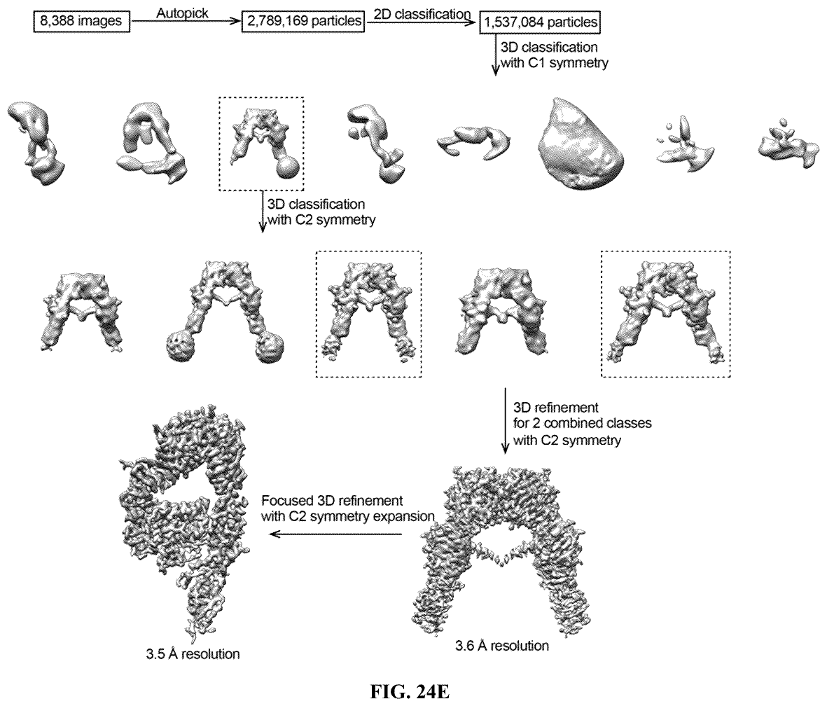

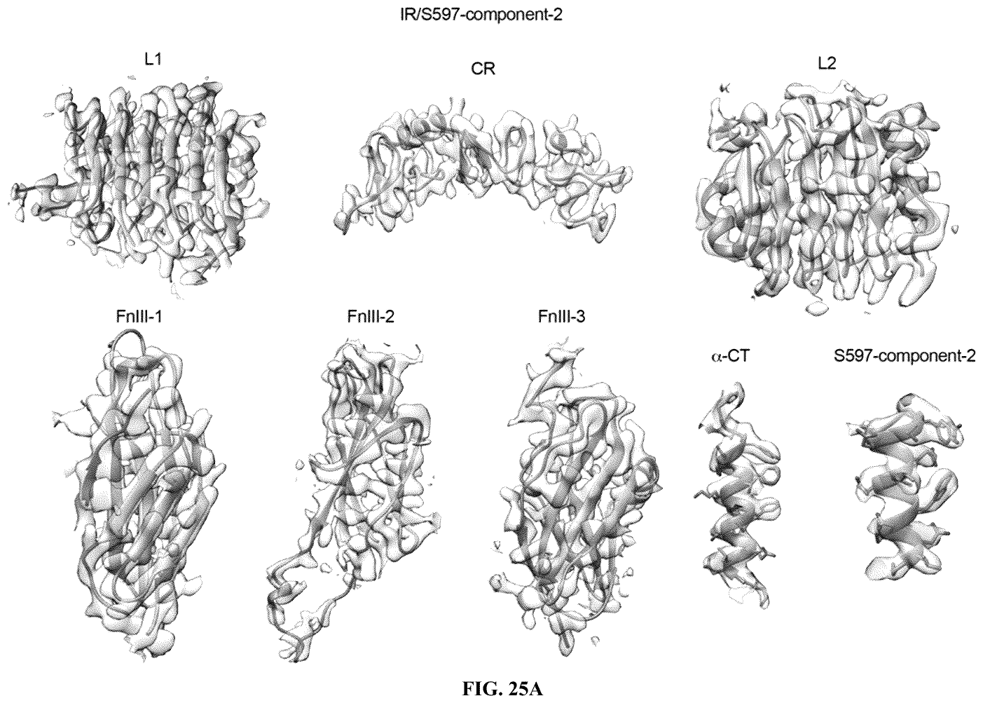

The binding pattern of S597-component-1 on the IR has been previously characterized by X-ray crystallography. In order to explore how the S597-component-2 engages the IR, the N-terminal 20 residues of S597 comprising only component-2 and the linker were synthesized (S597-N20, FIGS. 2A, 23B), and the cryo-EM structure of full-length mouse IR/S597-component-2 complex was determined at overall 3.6 Å resolution (FIGS. 2B, 13, 24, 25). The resolution for one-half of the IR/S597-component-2 complex was further improved to 3.5 Å resolution after symmetry expansion and focused refinement. Structural comparison between S597-component-2 bound IR and apo-IR revealed no major structural differences (FIG. 2C), suggesting that the S597-component-2 alone cannot induce the conformational change of IR required for signaling.

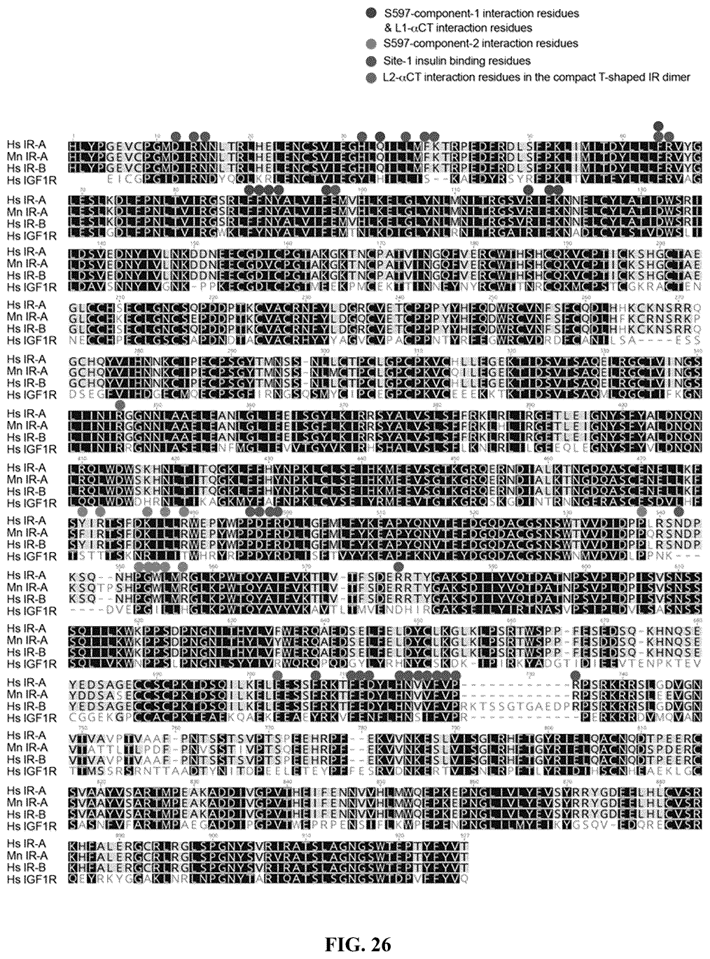

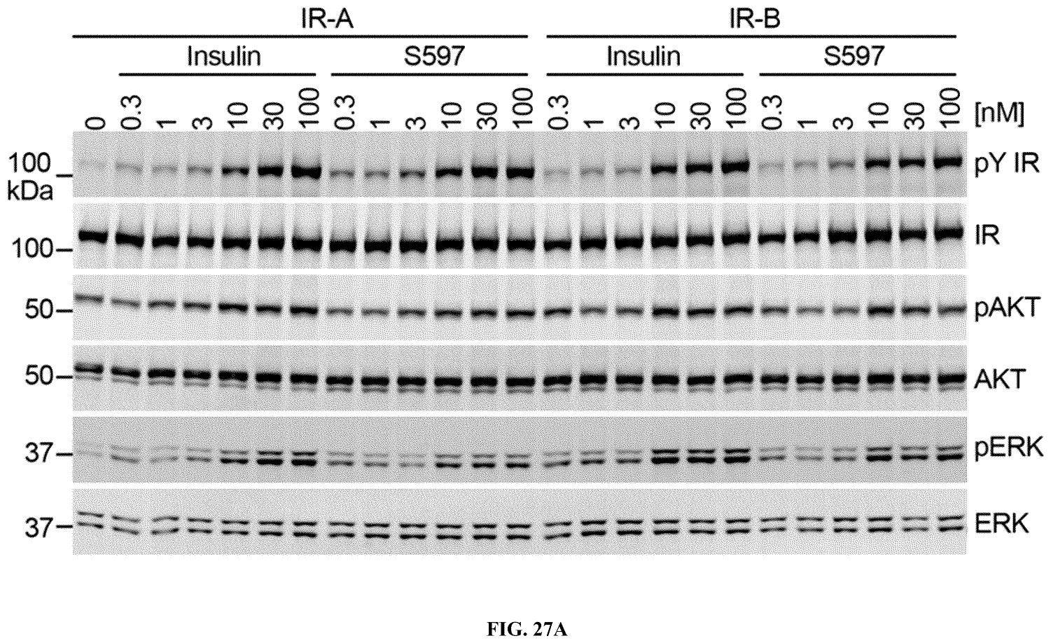

Additional strong densities were identified at the side-surface of the FnIII-1 domain of each IR protomer that could be unambiguously assigned to the S597-component-2 (FIGS. 2D, 2E). Fourteen residues that constitute the S597-component-2 could be built into cryo-EM map based on clear side-chain densities (FIGS. 2F-2G). The model shows that S597-component-2, assuming a short α-helical conformation, packs tightly against the side-surface of the FnIII-1 domain of IR mainly through hydrophobic interactions. Particularly, Leu2, Trp6, Ile9 and Tyr14 of S597-component-2 positioned along one side of the helix make strong interactions with several hydrophobic residues in the FnIII-1 domain of IR, including Leu486, Pro537, Pro549, Gly550 and Leu552. In addition, Glu5 of S597-component-2 and Arg16 of the linker region form salt bridges with Arg479 and Asp535 of the IR FnIII-1 domain, respectively, further contributing to the strong IR/S597-component-2 interaction. It is noteworthy that these key residues are conserved between human and mouse IR (FIG. 26).

b. Both Components-1 and -2 of S597 are Required for the IR Activation

It has been previously shown that the S597-component-1 binds the L1 domain of IR in a similar fashion as the α-CT motif (FIGS. 3B-3D). Therefore, the binding of S597-component-1 to the L1 domain of IR would disrupt the association between L1 domain and α-CT motif, which together serve as the insulin binding site-1. The new structure shows that the S597-component-2 binds to a similar surface on the FnIII-1 domain of IR as that used for the site-2 insulin binding (FIGS. 3E, 3F). Thus, the IR binding sites of S597-components-1 and -2 largely overlap with the insulin binding sites-1 and -2 on IR, respectively. Because the binding of multiple insulins to both site-1 and site-2 is required for optimal IR activation, it is likely that both the binding of S597-component-1 to the IR L1 domain and the binding of S597-component-2 to the IR FnIII-1 domain are required for the S597-induced IR activation.

To test this, F64A or F96A mutations were introduced into IR to disrupt the IR L1/S597-component-1 interaction. Because the A isoform of the IR was used for structure determination, and because there was no significant difference between the A and B isoforms of IR activation upon stimulation with S597 (FIGS. 27A, 27B), mutations were introduced into the A isoform of the IR. 293 FT cells expressing the IR F96 Å mutant exhibited great deficiency in S597-dependent IR activation (FIGS. 3G, 3H). However, the IR F64 Å mutant could still be activated by S597, suggesting that substitution of Phe64 with a small hydrophobic residue is not sufficient to disrupt the IR L1/S597 interaction (FIGS. 3G, 3H). The Phe64 and Phe96 of the L1 domain of IR form hydrophobic interactions with Phe705 and Phe701 in α-CT that are critical for insulin site-1 binding (FIG. 3C). As expected, both IR F64A and F96A mutants showed defective insulin-dependent activation (FIGS. 3G, 3H). These results confirm the importance of IR/S597-component-1 interaction in IR activation and also indicate that the L1/α-CT interaction, which is critical for insulin-dependent IR activation, is not required for S597-dependent IR activation (FIGS. 3G, 3H).

The action of S597 on the activation of an IR K484E/L552A double mutant was examined, which was expected to weaken both the IR/site-2 insulin and the IR/S597-component-2 interactions (FIGS. 3G, 3H). Consistent with the structural models, the IR K484E/L552A mutant could not be activated efficiently by either insulin or S597. Previous studies have shown that S597 does not activate the insulin-like growth factor 1 receptor (IGF1R), another receptor tyrosine kinase closely related to IR. Some key residues that are critical for IR/S597-component-2 interaction, including Tyr477, Arg479, Lys484, Arg488, Trp551, and Arg554, are not conserved in the IGF1R (FIGS. 2G, 26), partially explaining the specificity of S597 on IR activation.

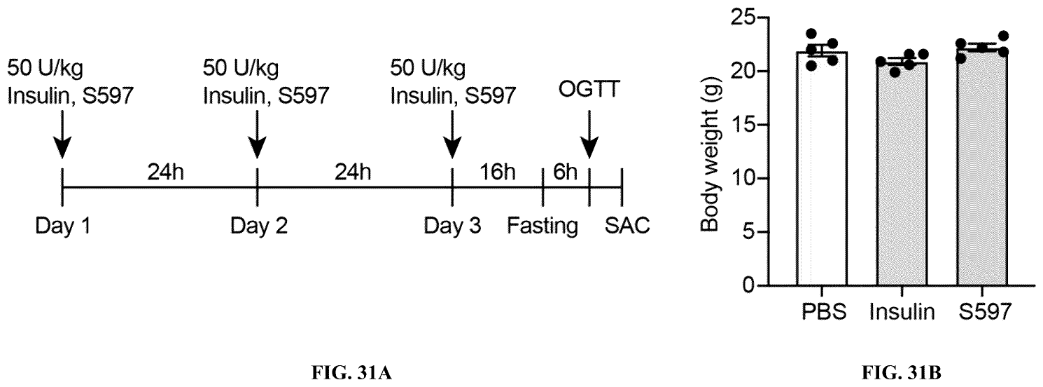

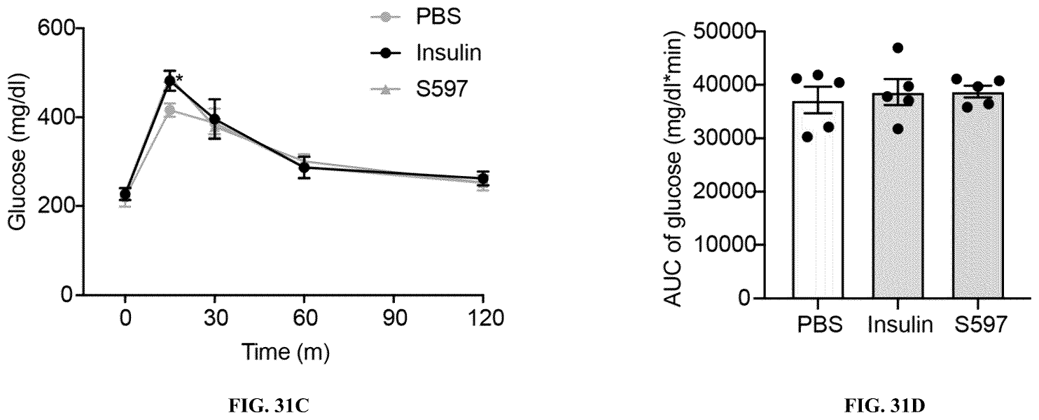

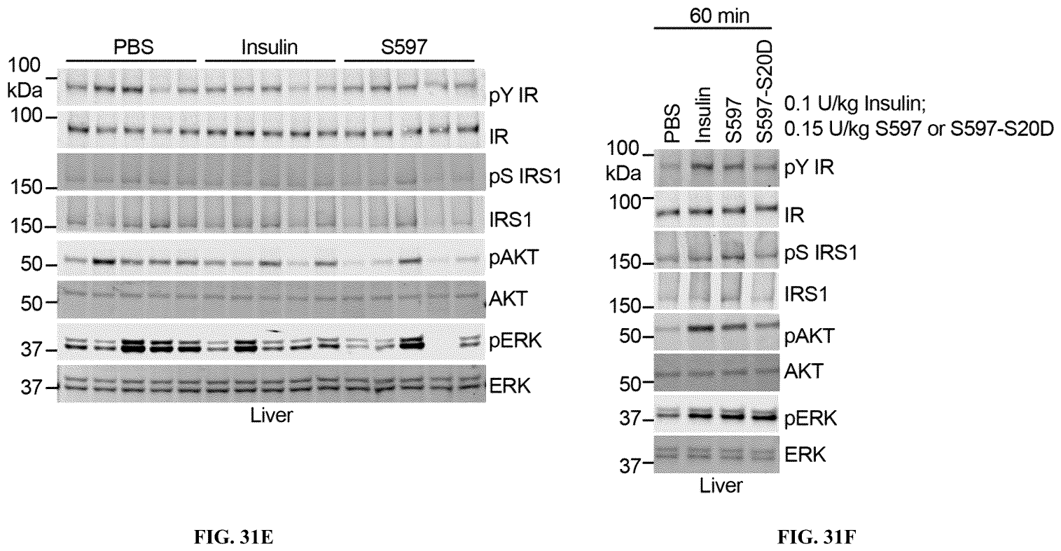

To further investigate the function and mechanism of S597 binding on IR activation, S597 was designed and synthesized with a mutation of the residue that is essential for binding to either L1 or FnIII-1 domains. Specifically, a S597-W6A mutation was introduced to disrupt the IR FnIII-1/S597-component-2 interaction and a S597-F27A mutation to interrupt the IR L1/S597-component-1 interaction (FIGS. 3A, 23B). Consistent with the results of IR mutants (FIGS. 3G, 3H), S597-W6A and -F27A mutants as well as S597-N20 showed greatly reduced potency in triggering IR activation relative to S597 and insulin (FIGS. 31, 3J). These data validate the functional importance of both L1/S597 and FnIII-1/S597 interactions for IR activation.

c. Structure of the Full-Length IR/S597 Complex

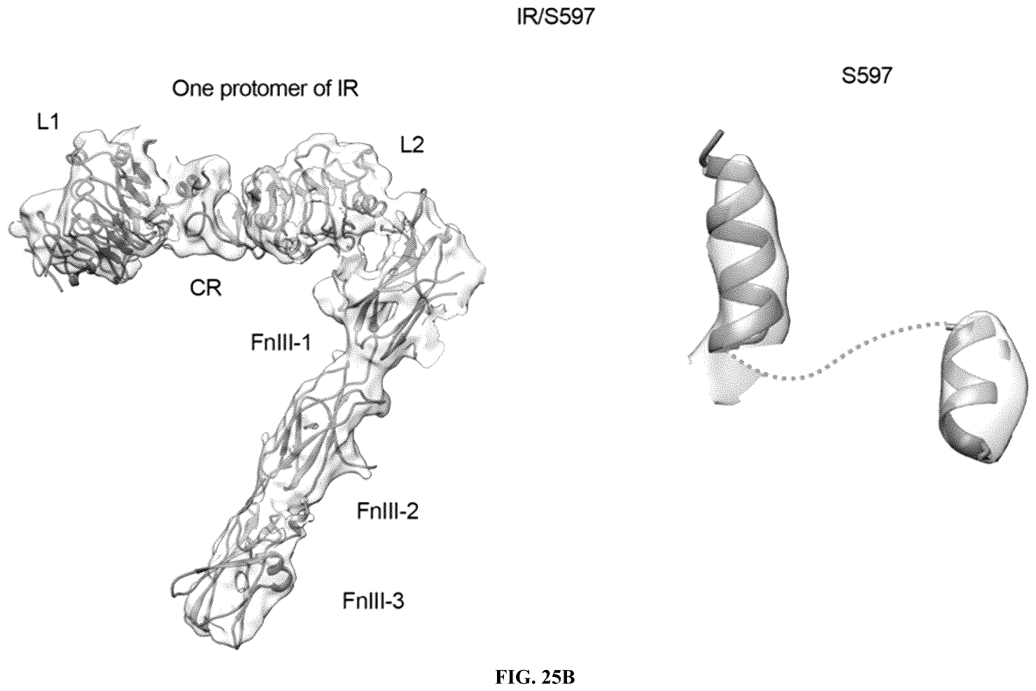

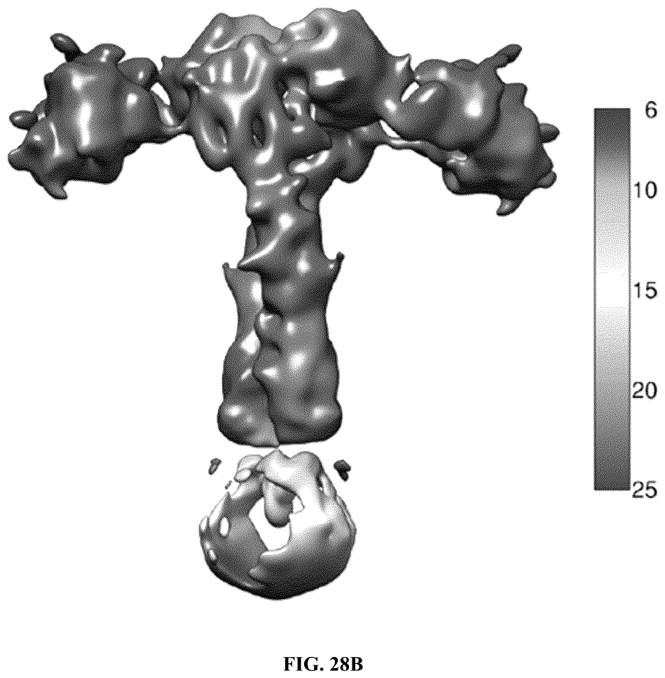

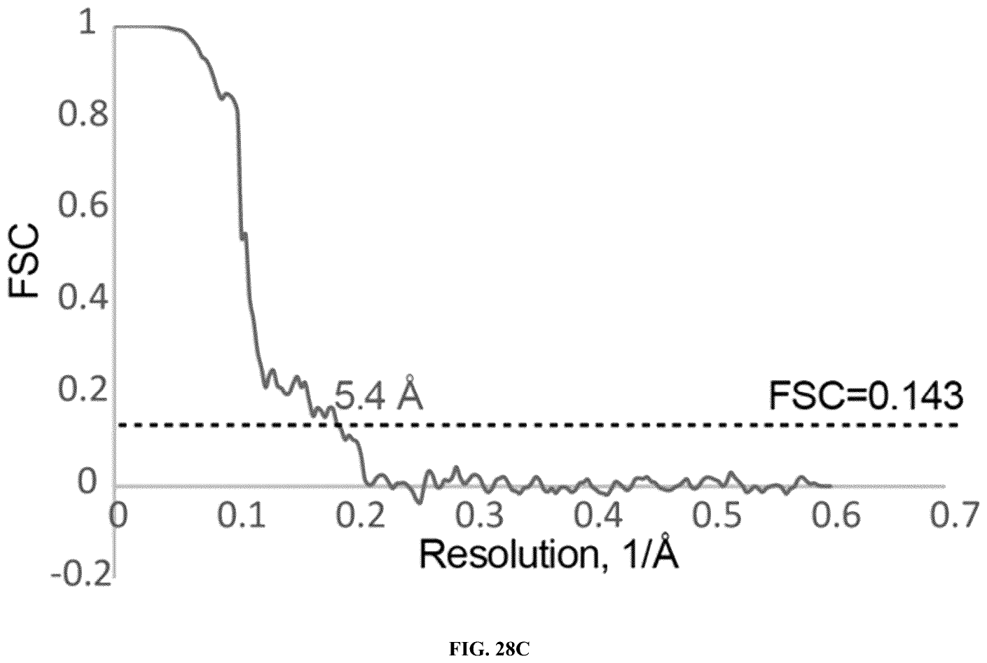

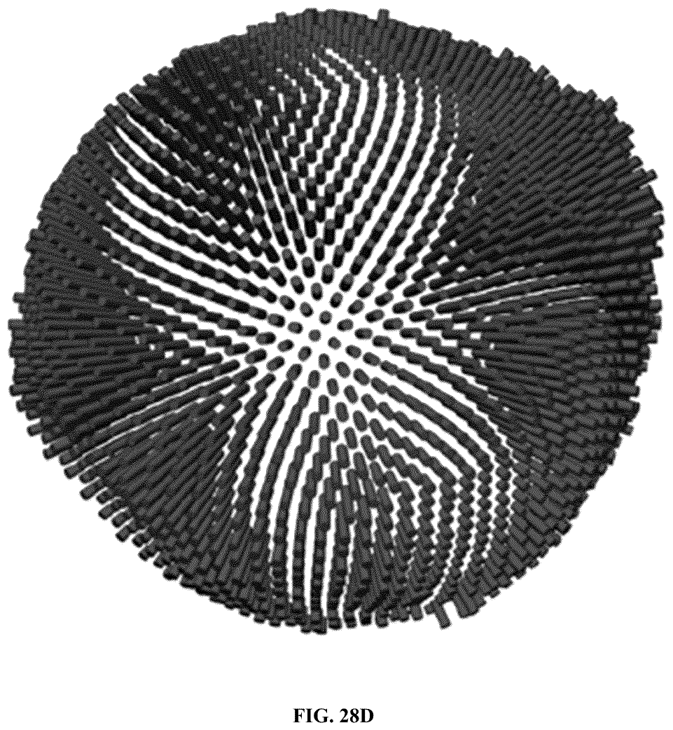

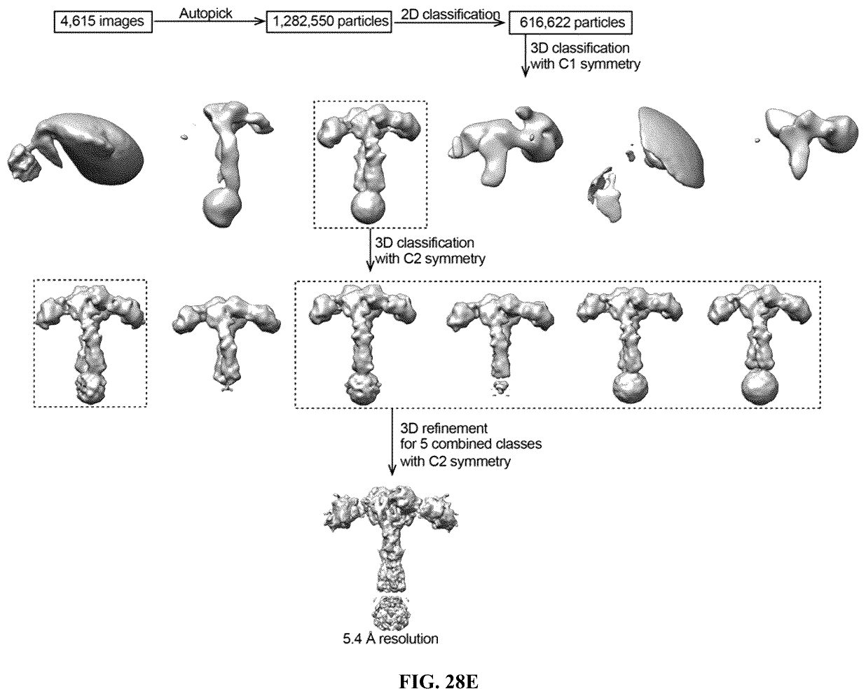

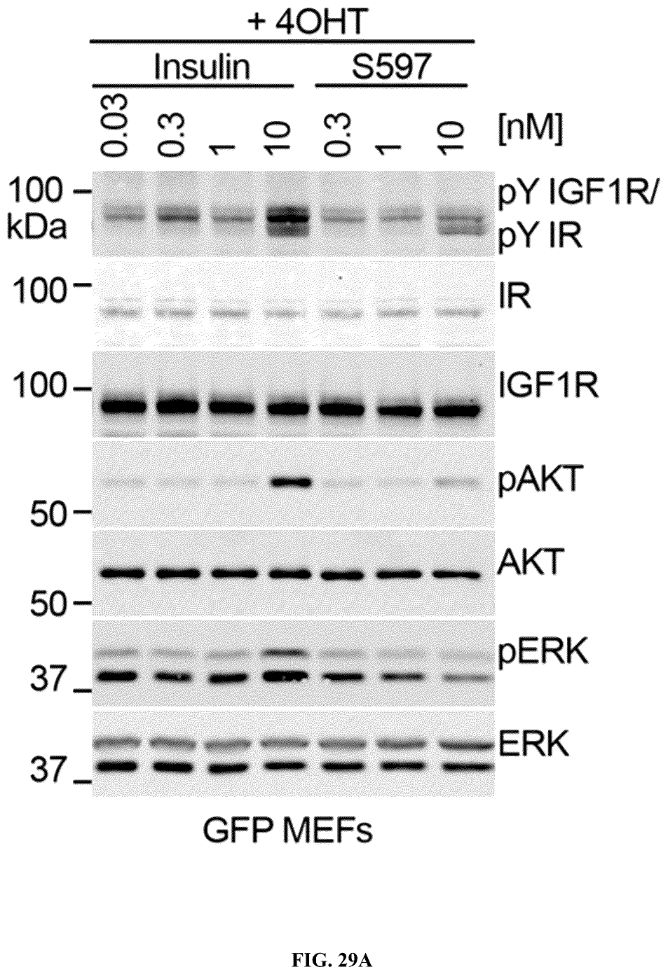

To reveal the molecular mechanism that underlies S597-induced IR activation, the cryo-EM structure of full-length IR in complex was determined using the complete S597 sequence comprising both components-1 and -2 as well as the linker connecting these two components (FIGS. 25, 28). The cryo-EM map of the IR/S597 complex was determined at 5.4 Å resolution, indicating the structural flexibility of this complex. Nonetheless, a model for the entire complex was built by rigid-body fitting the structures of the S597-component-1 bound L1 domain (determined previously by X-ray crystallography), the S597-component-2 bound FnIII-1 domain (determined in this work), as well as the other domains of IR (determined previously by cryo-EM) without major structural adjustments.

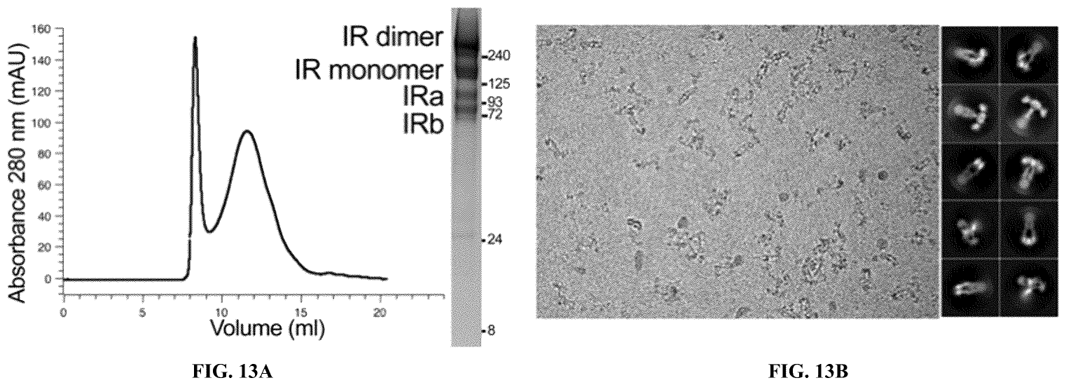

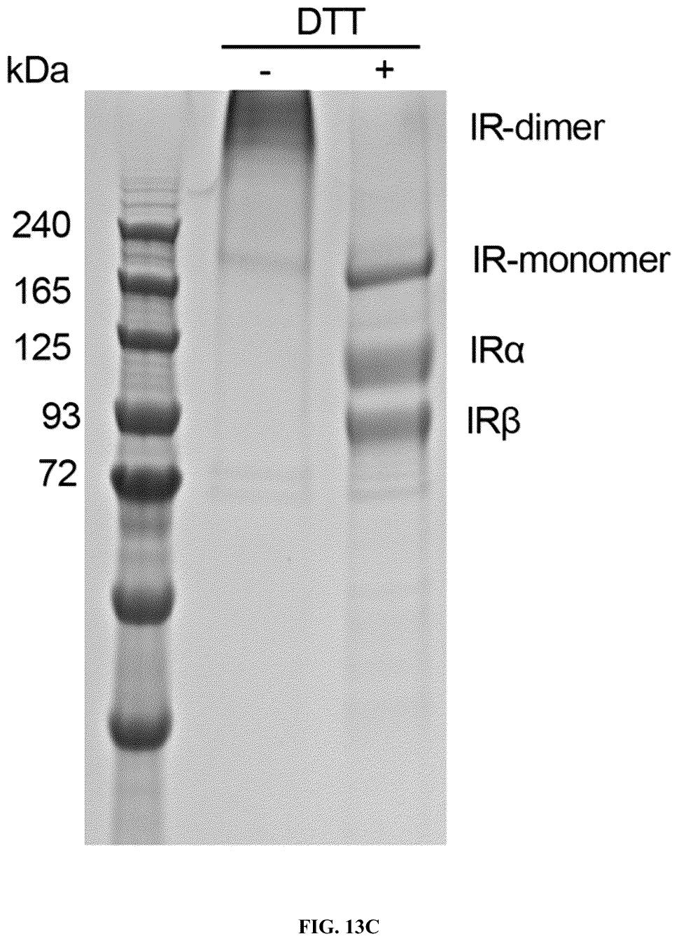

To increase the yield of full-length IR, the key residues in the IR that are important for IR endocytosis were mutated and the Tse3-Tsi3 affinity purification system was employed. The protein was eluted as a sharp peak in size exclusion chromatography (SEC) and ran as a discrete band on SDS-PAGE (FIG. 13A). The purified full-length IR was mixed with S597, which was then subject to cryo-EM analysis. Cryo-EM micrographs showed monodispersed particles, and the 2D class averages were strikingly different from that of IR/insulin complexes (FIG. 13B), suggesting that S597 activates IR in a different manner to insulin.

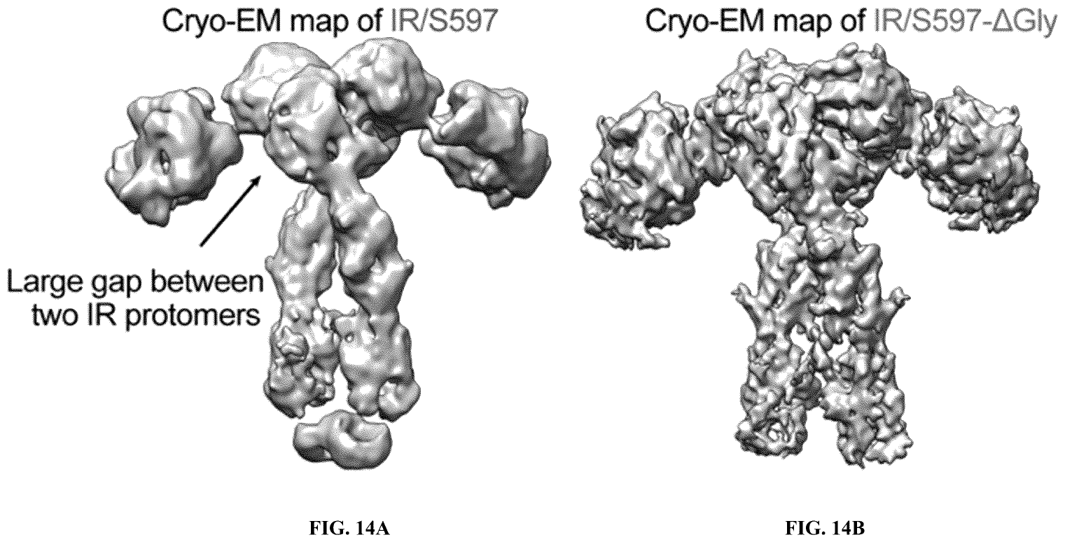

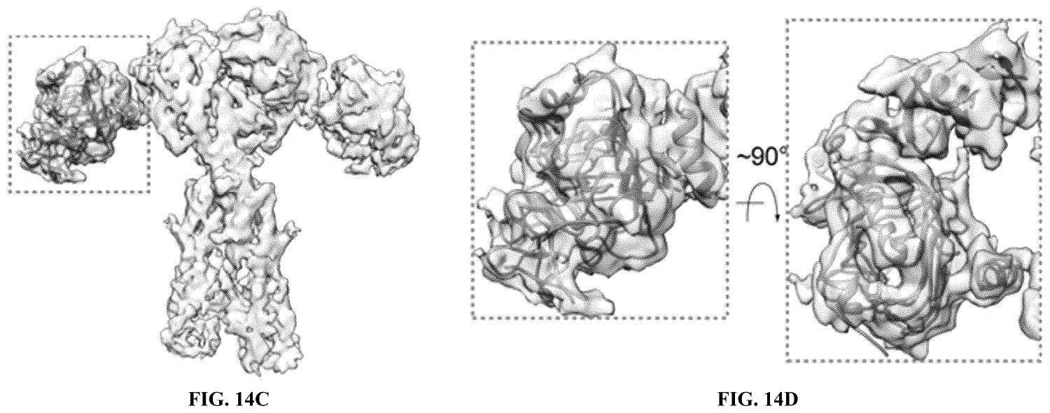

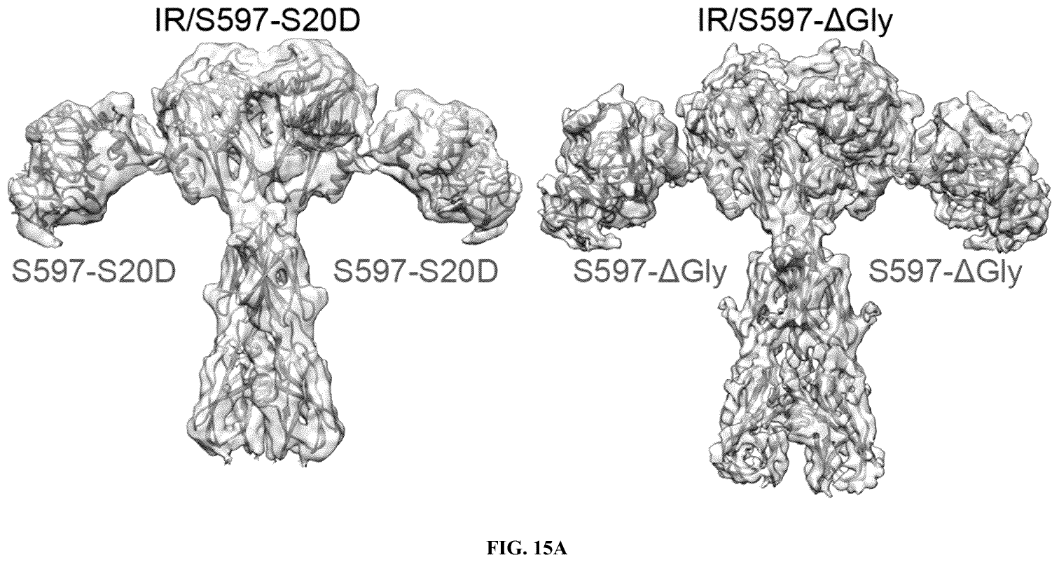

In contrast to the IR/S597-component-2 complex, the structure of IR/S597 assumes an extended T-shaped symmetric conformation, bringing together the two membrane-proximal stalks (FIGS. 4A, 4B). This structural feature suggests that IR bound with the full-length S597 represents the active ligand-receptor complex. Two S597 molecules were observed in the top region of the extended T-shaped IR. Each S597, which adopts a helix-loop-helix fold, is embraced between two IR protomers at the top part of the extended-T. The crystal structure of IR L1/CR domains in complex with the S597-component-1 and the new cryo-EM structure of IR FnIII-1 domain in complex with the S597-component-2 can be fit into the cryo-EM density as a rigid body (FIGS. 14C-14D), indicating that (1) one of two helices in the helix-loop-helix motif could be attributed to the S597-component-1; (2) the S597-component-1 engages the full-length IR in an identical manner to the isolated L1/CR domains of IR. The S597-component-1 contacts the L1 domain of one IR protomer, while the component-2 from the same S597 interacts with the FnIII-1 domain of the adjacent protomer (FIG. 4C). In this way, one S597 molecule simultaneously engages the two IR protomers, thereby stabilizing the active conformation of IR. The linker connecting the site-1 and site-2 components of IR that consists of six residues was not resolved in the cryo-EM map, reflecting its structural flexibility. The dynamic nature of S597 likely contributes to the structural flexibility of the entire IR/S597 complex, partially explaining why the cryo-EM map of the IR/S597 complex was limited in resolution. In addition to the S597-mediated protomer-protomer interaction, the L2 domain of one protomer also interacts weakly with the FnIII-1 domain of another, further maintaining the extended T-shaped active conformation of IR (FIGS. 4A, 4B).

The S597-component-1 competes for binding to the same site (i.e. IR L1) with αCT, thus delocalizing the αCT from the IR L1 and destabilizing the auto-inhibitory conformation of IR. Strikingly, the cryo-EM map for the first time shows that the S597-component-2, which is folded as a single α-helix, contacts a side surface of FnIII-1 domain of adjacent IR protomer (FIG. 4B). Notably, a similar FnIII-1 surface is also used for site-2 insulin binding, but their binding patterns differ remarkably. Our model shows that the S597-component-1 competes for binding to the same site (i.e., IR L1) with αCT, while the S597-component-2 contacts a side surface of FnIII-1 domain of adjacent IR protomer. With this specific binding mode, S597 crosslinks two IR protomers by simultaneously contacting the L1 domain of one protomer and the FnIII-1 domain of the other (FIG. 4C), thus stabilizing the ‘extended T’-shape.

These structural comparisons reveal that the conformation of one protomer in this extended T-shaped IR closely resembles that of the protomer in the A-shaped apo-IR dimer (FIG. 4D). This structural analysis strongly suggests that, after the apo-state of IR is disrupted by the binding of S597-component-1 to the L1 domain of IR, the two protomers undergo a rigid-body rotation using the L2-FnIII-1 interface as a hinge, leading to the extended T-shaped IR (FIG. 4E). This activation mechanism is distinct from the insulin-induced IR activation that involves both intra-protomer motion and inter-protomer rotation. Furthermore, S597 stabilizes the active conformation of IR in a distinct manner from insulin. Specifically, in the T-shaped active IR/insulin complex, the site-1 insulin, which primarily binds to the L1/α-CT of one protomer, also contacts the top surface of the FnIII-1 domain of another. In this way, the binding of two insulins to site-1 of IR brings the L1 domains on the top of the FnIII-1 domains, thus triggering a compact T-shaped IR conformation. Two additional insulins bind to the side-surface of the FnIII-1 domains of IR, known as site-2. However, the site-2 insulins do not play major roles in maintaining the active conformation of IR, as they only contact one protomer. In contrast to the 4:1 (insulin: IR dimer) IR/insulin complex, only two S597s are bound to the IR dimer in the structure of the IR/S597 complex. Each S597 molecule simultaneously interacts with both the L1 domain and the side surface of the FnIII-1 domain; such that, the L1 domain of one protomer is placed on the lateral side of the FnIII-1 domain of the adjacent protomer, leading to a more extended T-shaped active conformation.

### Example 2. Structure of the Full-Length IR/S597 Analog Complex

To understand how the S597-component-2 engages the IR, the N-terminal 20 residues of S597 containing only the S597-component-2 were synthesized (FIG. 12A) and the cryo-EM structure of full-length IR/S597-component-2 complex at overall 3.6 Å resolution was determined (FIG. 2B). The resolution for one-half of the IR/S597-component-2 complex was further improved to 3.5 Å resolution after symmetry expansion and focused refinement (FIGS. 2D, 2E). Consistent with the structure of IR/S597, additional strong densities were identified at the side-surface of the FnIII-1 domain of each IR protomer that could be unambiguously assigned to the S597-component-2. 14 residues that constitute the S597-component-2 could be built into cryo-EM map based on clear side-chain densities. The model shows that the S597-component-2, assuming a short α-helical conformation, packs tightly against the side-surface of the FnIII-1 domain of IR mainly through hydrophobic interactions (FIG. 2G). In addition, Glu5 of S597-component-2 and Arg16 of the linker region form salt bridges with Arg479 and Asp535 of the IR FnIII-1, respectively, further contributing to the IR/S597-component-2 interaction.

Structural comparison between apo-IR and S597-component-2 bound IR revealed no major structural differences, suggesting that the S597-component-2 alone cannot induce the conformational change of IR required for signaling. Nevertheless, the overall structure of the IR/S597 complex has an ‘extended T’-shape, significantly different to that of apo-IR and the insulin induced active-IR (FIG. 4). Importantly, the distance between the two membrane-proximal FnIII-3 domains of IR in this structure is ˜36 Å, hinting that the two TM domains of IR are positioned in proximity in this ‘extended T’-shape that allows the intracellular kinases to undergo trans-autophosphorylation. These structural features suggest that the ‘extended T’-shape represents an active state of IR which is unique to that induced by insulin. Structural comparisons reveal that the conformation of one protomer in the ‘extended T’-shape resembles that of the protomer in the ‘Λ’-shaped apo-IR (FIG. 4D), indicating that, after the apo-IR is disrupted by the binding of S597-component-1 to IR L1, two protomers will undergo a scissor-like rotation using the interface between two L2 domains as the hinge, leading to the ‘extended T’-shape IR (FIG. 4F). The ‘extended T’-shaped IR is maintained by S597-mediated crosslinking of two protomers.

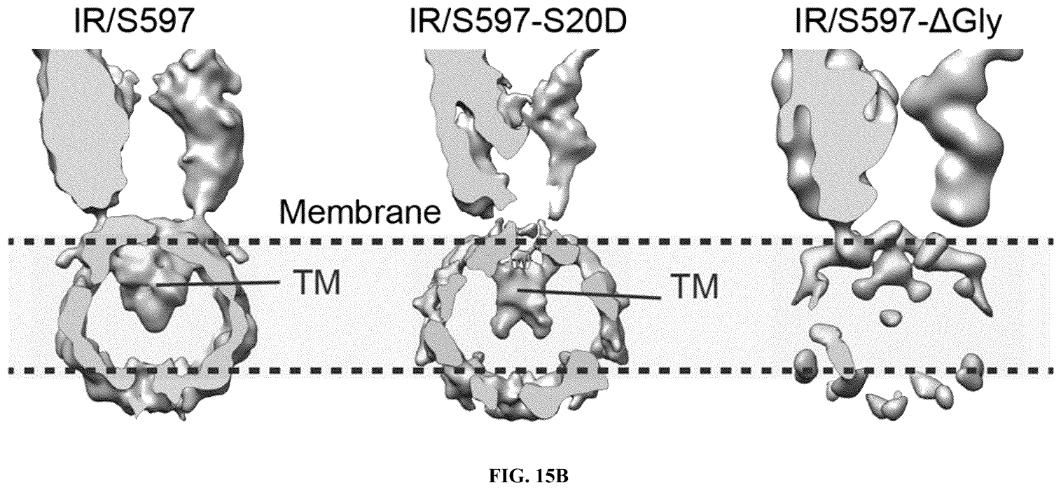

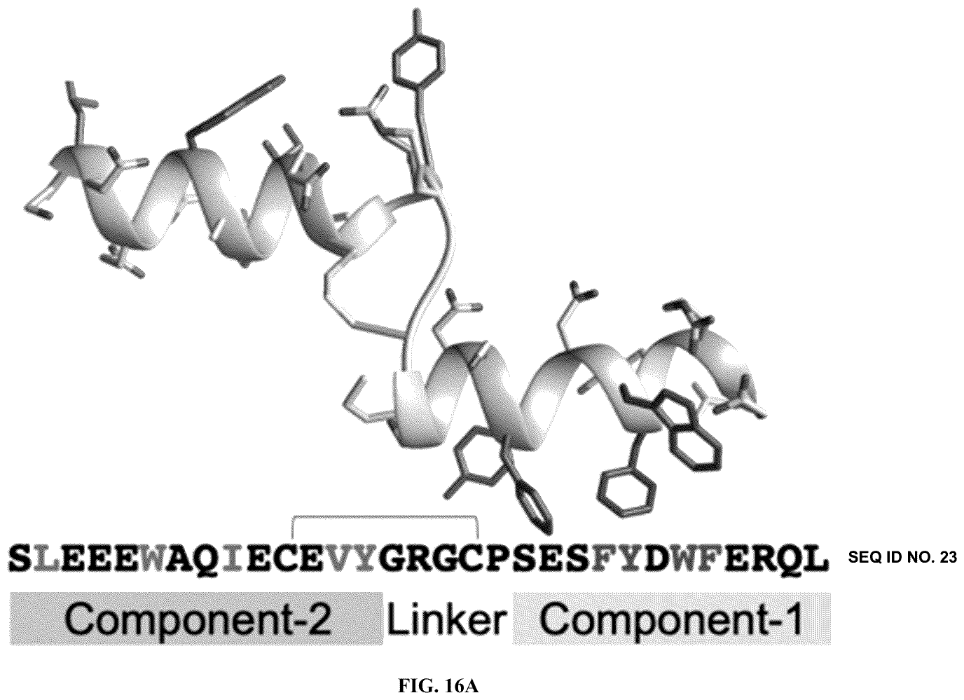

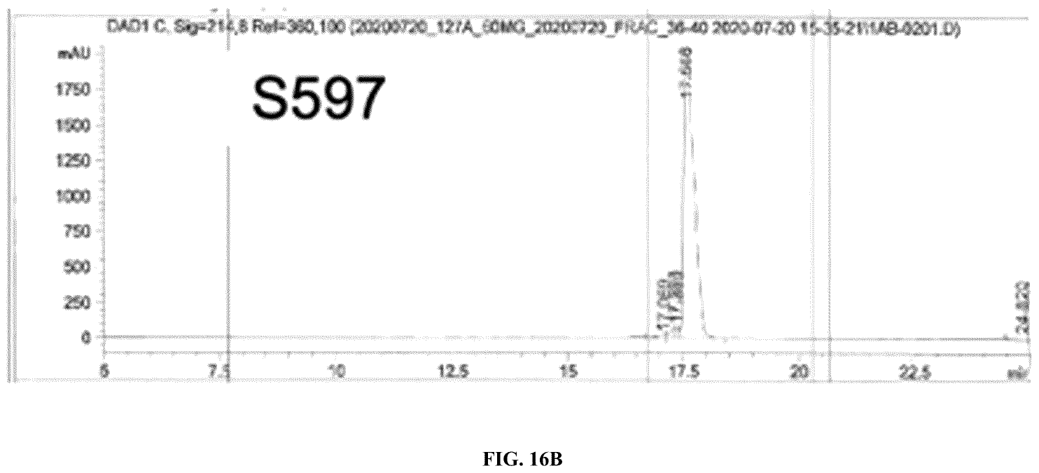

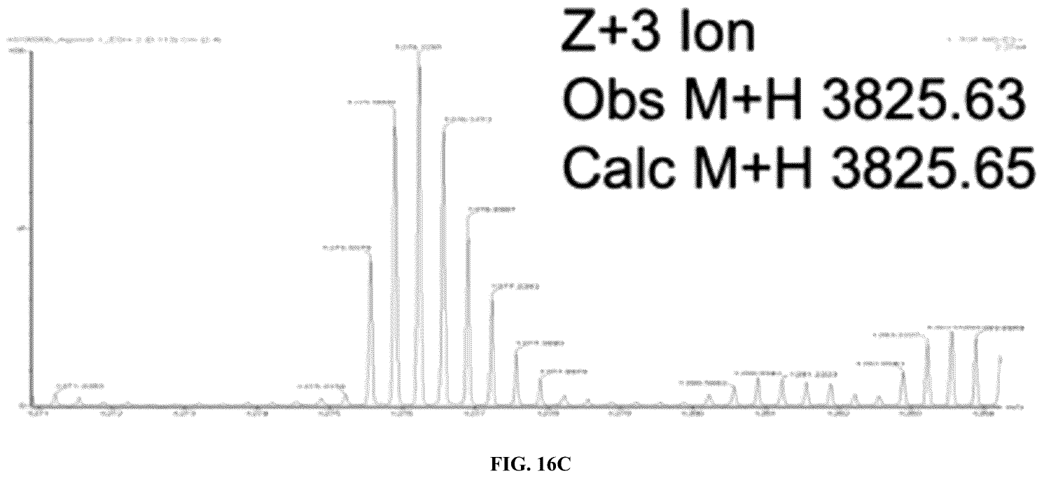

Interestingly, the structures of the extracellular regions of IR/S597-S20D and IR/S597-ΔGly closely resemble that of IR/S597 (FIG. 15A). However, as observed after further 3D classification, the TM domains of IR/S597 and IR/S597-S20D adopt rather different conformations, which suggests that IR/S597 and IR/S597-S20D may generate distinct biased signaling through allosterically modulating the conformation of TM domains (FIG. 15B). While S597-ΔGly provided enhanced rigidity and improved cryo-EM data quality, this mimetic was unexpectedly deficient in IR signaling. In addition, the TM domain of IR/S597-ΔGly is highly disordered, partly explaining the low potency of S597-ΔGly (FIGS. 6B, 15B).

### Example 3. S597 Activates Disease-Causing IR Mutants

Certain mutations of IR cause inherited severe insulin resistance syndromes. Because S597 binds and activates IR in a different manner from insulin, whether S597 could activate those disease-causing IR mutants, which are predicted to be defective in insulin binding, was explored.

IR site-1 binding-deficient mutations that are found in the L1 (R14W, N15K), FnIII-1 loop (D496N), and α-CT (D707A) domains (FIGS. 5A, 5B, 26), all of which are not localized at IR/S597 interfaces, were selected. The Asp496 in the IR was mutated to lysine as the charge reversion mutation was expected to disrupt insulin-binding. The Arg14 in the IR was mutated to either alanine or tryptophan. In addition to these disease-causing IR mutants, several other key residues were mutated that are important for insulin binding but not S597 binding, including Phe497 in the loop of the FnIII-1 domain and Phe701/Phe705 of the α-CT motif (which contact the L1 domain) (FIGS. 5C, 5D, 27C). IR R14W, R14A, N15K, D707A, D496K and F701A/F705A showed expectedly reduced insulin-dependent IR activation, confirming their deficiency in insulin binding (FIGS. 5C, 5F, 27C). IR F497A can be activated by insulin but with less efficiency than IR WT. Nevertheless, all the above IR mutants could be fully activated by S597 (FIGS. 5C, 5F, 27C)

Insulin bound IR undergoes a large conformational change between the CR and L2 domains, and between L2 and FnIII-1 domains. These changes generate new intra- and inter-domain contacts, thus stabilizing compact T-shaped IR. Due to the distinct activation mechanism of IR upon S597 binding, S597 could activate IR mutants that disrupt stabilization of the compact T-shaped IR upon insulin binding. To test this, two residues Arg345 (L2 domain) and Glu697 (N-terminal region of α-CT) in the IR were selected, which form a salt bridge in the insulin-induced compact T-shaped IR. Mutation of IR R345A or E697A largely diminished IR activation by insulin, but those mutants could be fully activated by S597 (FIGS. 5E-5F). These results confirm the structural observation that S597 uniquely engages and activates IR, and suggest that S597 activates disease-causing and insulin binding-deficient mutants of IR, as well as mutants that disrupt the stability of the compact T-shaped IR.

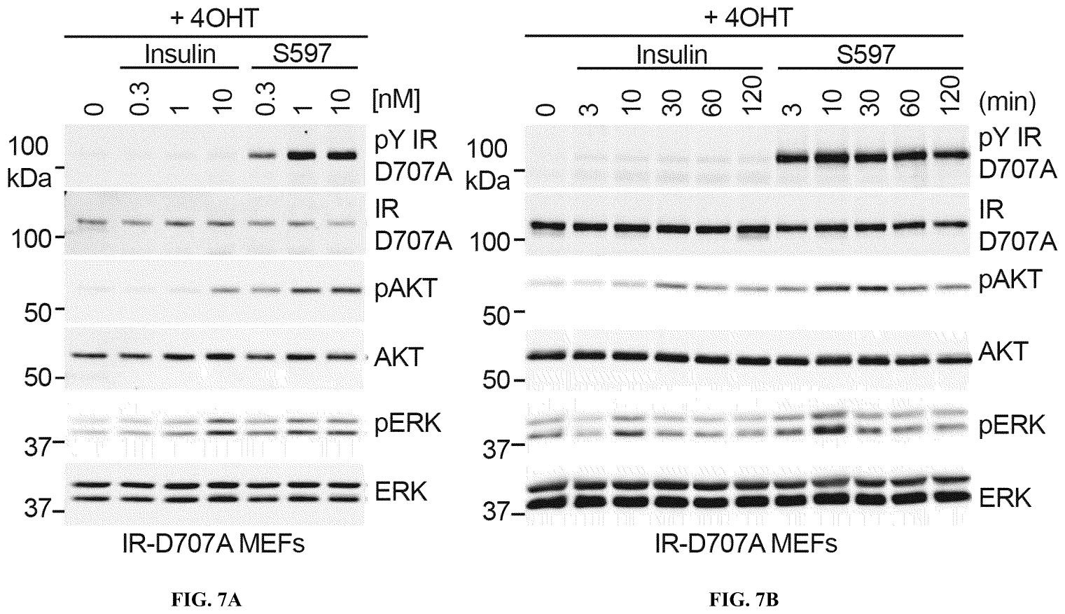

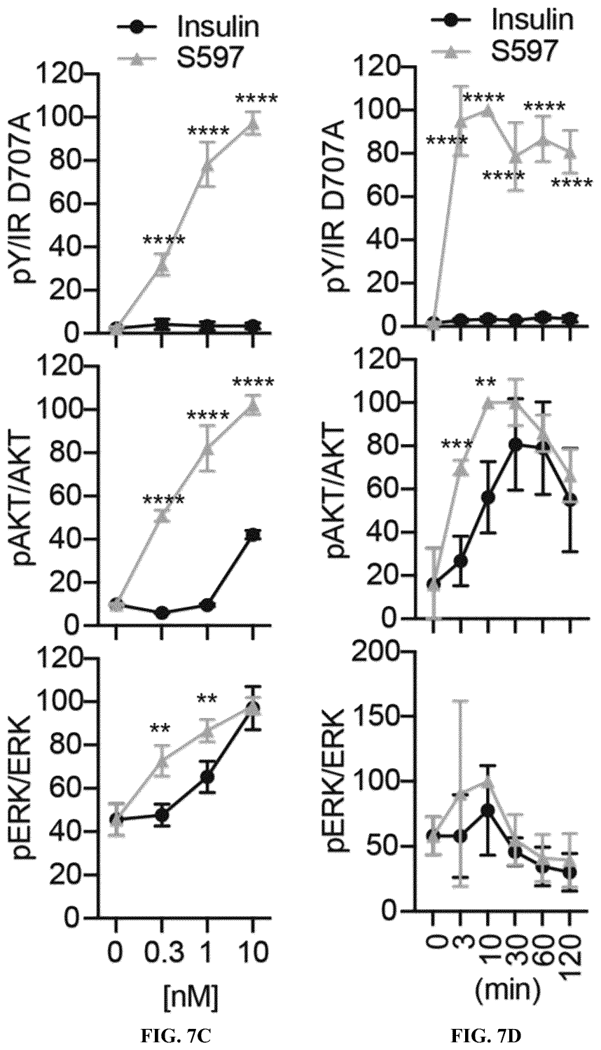

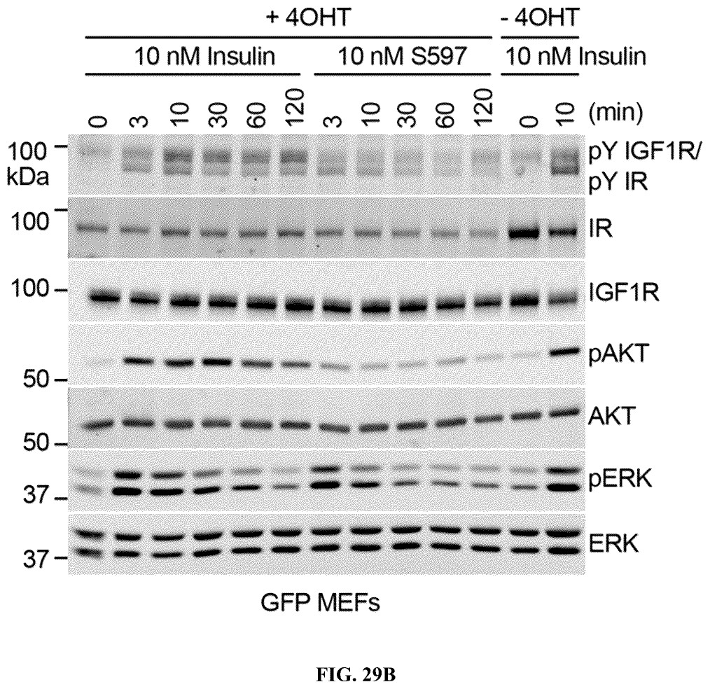

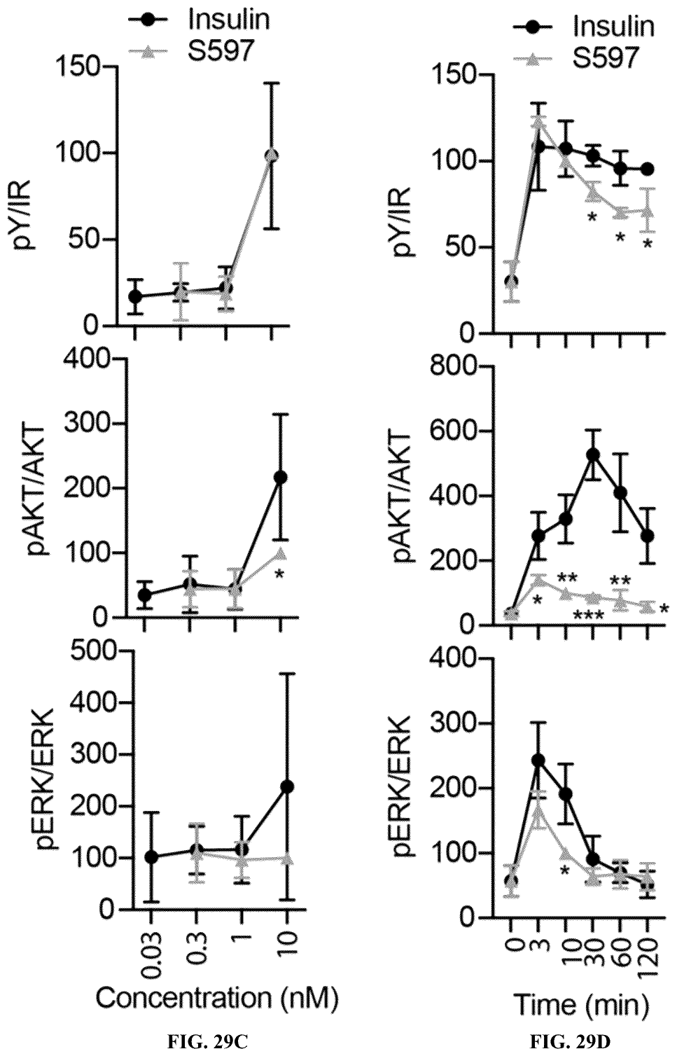

In order to fully characterize the action between insulin and S597 in IR signaling, a cell-based model of severe insulin resistance was created using mouse embryonic fibroblasts (MEFs) derived from tamoxifen-inducible IR knockout (IRF/F; Cre-ERT, Jackson #004682, #006955, C57BL/6). IR D707A tagged with the C-terminal green fluorescence protein (GFP) or GFP alone was introduced into IR conditional knockout mouse embryonic fibroblasts (IR-D707A MEFs and GFP MEFs, respectively). The cells were treated with tamoxifen to remove endogenous murine IR and monitored auto-phosphorylation of IR (pY1152/1153; equivalent to pY1150/1151 of human IR, pY IR), AKT (pT308, pAKT), and ERK1/2 (pT202/Y204, pERK) over a wide range of insulin concentrations (FIGS. 7A, 7C, 29A, 29C) and at multiple time points (FIGS. 7B, 7D, 29B, 29D). S597, but not insulin, significantly increased the levels of pY IR, pAKT, and pERK in the MEFs expressing insulin binding-deficient IR (FIGS. 7A, 7C).

Specifically, tamoxifen treatment greatly reduced endogenous murine IR levels, but the residual IR could activate downstream signaling upon high concentration of insulin stimulation (FIG. 29B). The expression levels of IGF1R were not changed by tamoxifen treatment (FIGS. 29A, 29B). The C-terminal GFP tag of IR D707A enabled the size shift of the IR D707A on the blots to distinguish endogenous murine pY IR and pY IGF1R from ectopic human pY IR D707A (FIGS. 7A-7B). Consistent with the results in 293 FT cells expressing IR D707A, insulin-triggered phosphorylation of IR was remarkedly reduced in IR-D707A MEFs (FIGS. 7A-7D). Consequently, the level of phosphorylation of AKT and ERK was greatly reduced in insulin-treated IR-D707A MEFs, indicative of defective IR signaling. High concentration of insulin significantly increased pAKT and pERK in IR-D707A MEFs potentially through the residual IR or crosstalk with IGF1R. In sharp contrast, S597 significantly increased the level of phosphorylation of IR, AKT, and ERK in IR-D707A MEFs over a wide range of concentrations (FIGS. 7A, 7C). These results suggest that S597 or other similar mimetics could activate the IR signaling in patients with insulin binding-deficient IR mutants. These data suggest that designed insulin mimetics activate IR signaling in patients with insulin binding-deficient IR mutants and provided a viable therapeutic intervention.

### Example 4. S597 Selectively Activates IR Signaling within Different Tissues

The insulin-activated IR triggers two major signaling pathways: the PI3K-AKT pathway and the MAPK pathway that control metabolism, and cell growth and proliferation (FIG. 9A). An initial cryo-EM structure of IR/S597 also showed that S597 engages and activates IR in a different manner to insulin. Strikingly, S597-bound IR activates AKT pathways as effectively as insulin but is less effective in activating MAPK pathways.

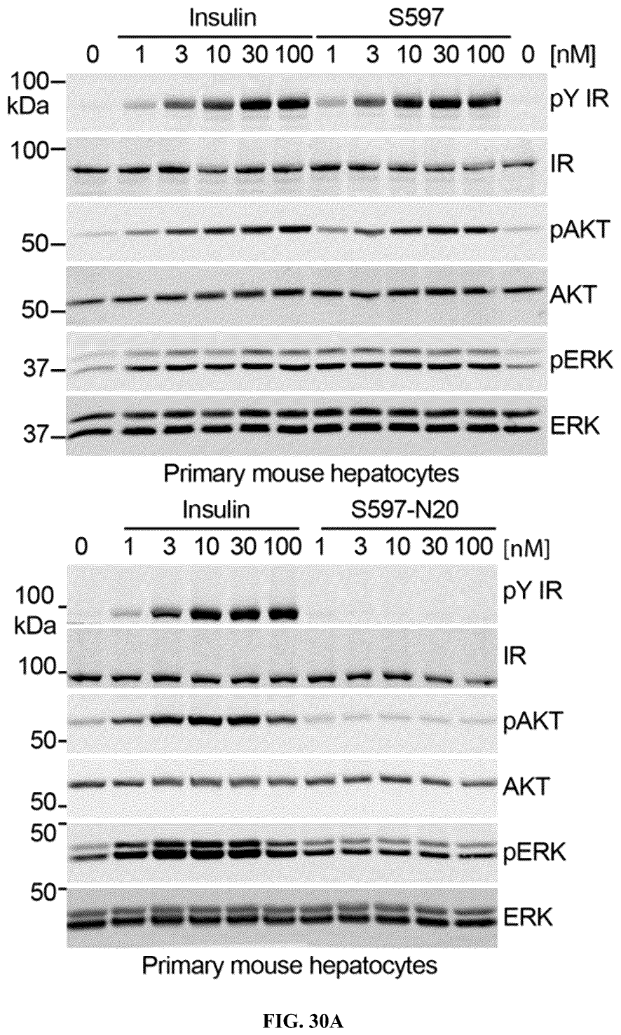

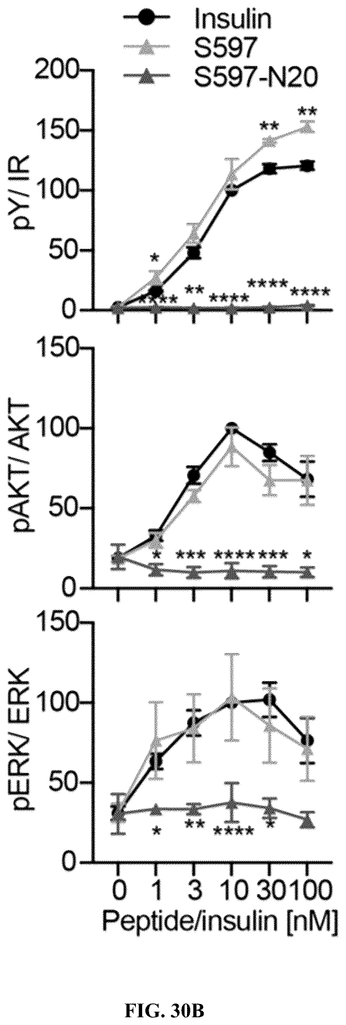

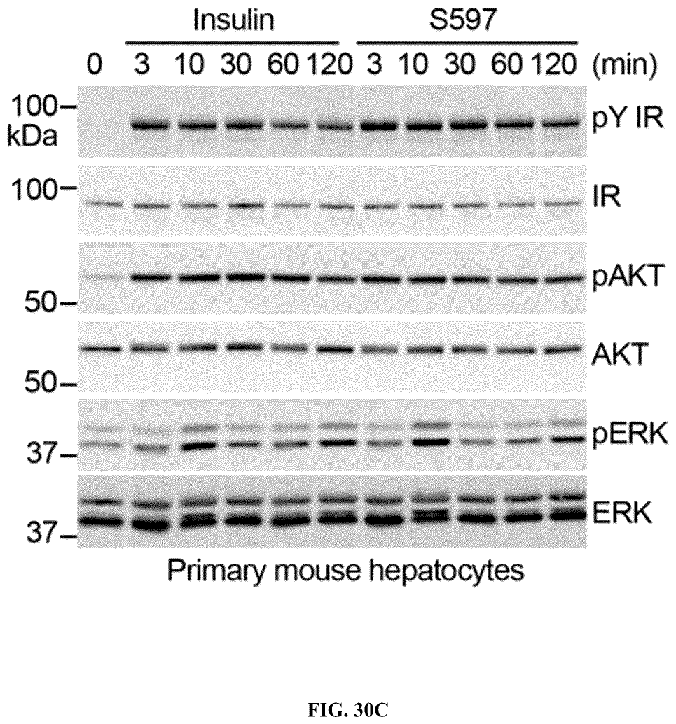

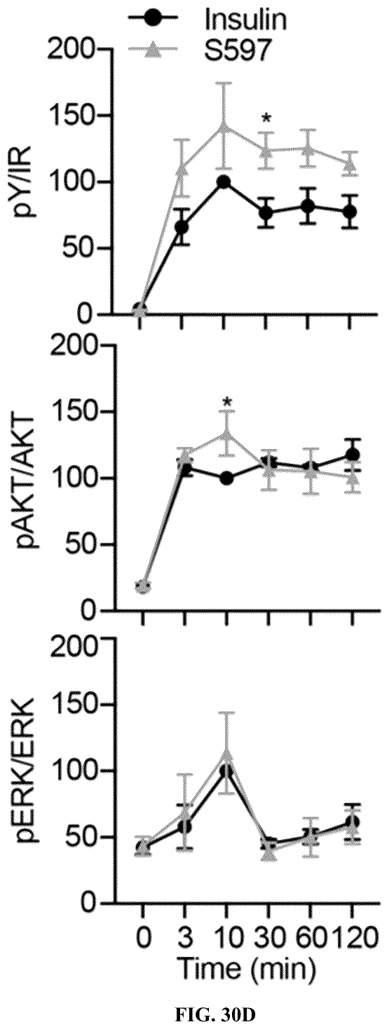

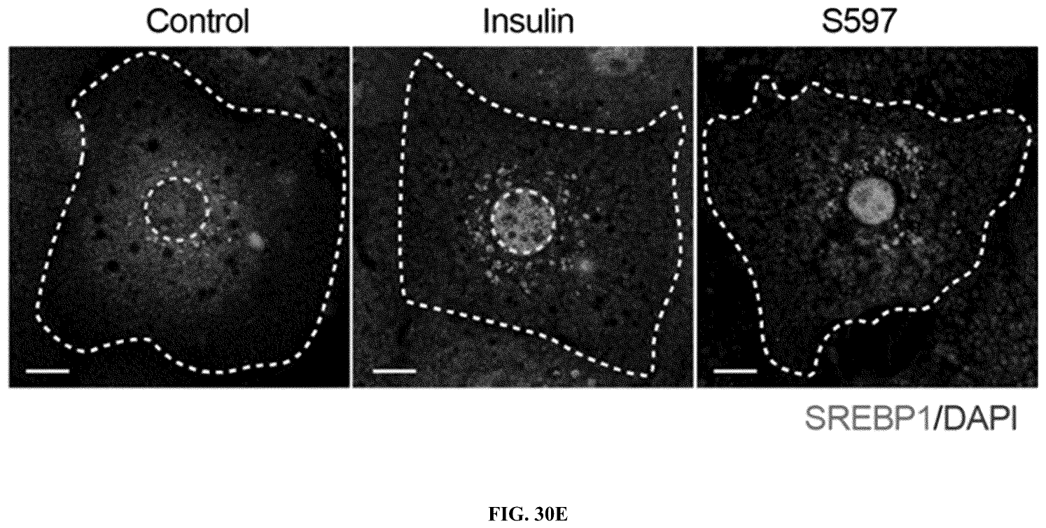

In 3T3-L1 and IR-transfected L6 myoblasts, S597 selectively activates PI3K-AKT pathway while leaving the MAPK pathway largely inactive. The effect of S597 on IR signaling was also examined in the liver, epididymal white adipose tissue (WAT), and skeletal muscle of mice (FIG. 6B). Intriguingly, while S597 significantly increased the levels of pY IR and pAKT in skeletal muscle, S597 was less potent in stimulating pERK (FIGS. 8B, 8C). In the liver and WAT, S597 induced robust auto-phosphorylation of the IR, albeit with lower potency than insulin. In contrast to the biased signaling observed in skeletal muscle, in liver, S597 was able to increase both pAKT and pERK to a similar levels as insulin (FIGS. 8B, 8C). In the WAT, both insulin and S597 significantly increased pAKT levels, while neither insulin nor S597 had a statistically significant effect on pERK levels, compared to vehicle treated conditions. Moreover, similar to insulin treatment, S597 promoted IR internalization in the liver (FIGS. 8D, 9B). Primary mouse hepatocytes were isolated and insulin- or S597-induced IR signaling over a wide range of insulin concentrations (FIGS. 30A, 30B) and at multiple time points (FIGS. 30C-30D) was examined. As expected, in primary hepatocytes, S597-N20 (S597-component-2 alone) did not induce phosphorylation of IR, AKT and ERK; while both S597 and insulin increased the levels of pY IR, pAKT, and pERK similarly, consistent with the in vivo data (FIGS. 30A, 30B). Thus, S597 selectively activates the PI3K-AKT pathway, leaving the MAPK pathway inactive in blood leukocytes, skeletal muscles, and adipose tissues of mice.

Quantitative phosphoproteomics of skeletal muscle showed that S597 and insulin phosphoprotcomes differ despite many similarities in signaling. Particularly, phosphorylation levels of upstream MAPK pathway proteins (e.g., SOS1, SHP2) were significantly reduced in S597-treated samples (FIG. 6C). This is consistent with the results that S597 may specifically activate the metabolic response through the IR-PI3K-AKT axis. The discrepancies in actions of S597 might suggest that insulin mimetics can induce distinct signaling pathways after IR activation in different insulin target tissues (FIG. 9A).

Insulin promotes nuclear translocation of sterol regulatory element-binding protein 1 (SREBP1) and stimulates hepatic lipogenesis. Thus, the cellular localization of SREBP1 was analyzed in the absence or presence of S597 in primary hepatocytes (FIGS. 8E, 30E). The nuclear translocation of SREBP1 was stimulated to the same level by insulin and S597, suggesting that S597 can activate SREBP1 function in the liver as well. These findings together with the study in Zucker diabetic fatty rats suggest that S597 promotes hepatic de novo lipid synthesis like insulin.

### Example 5. S597 Analogs Lower Blood Glucose but not Cell Proliferation as Efficiently as Insulin

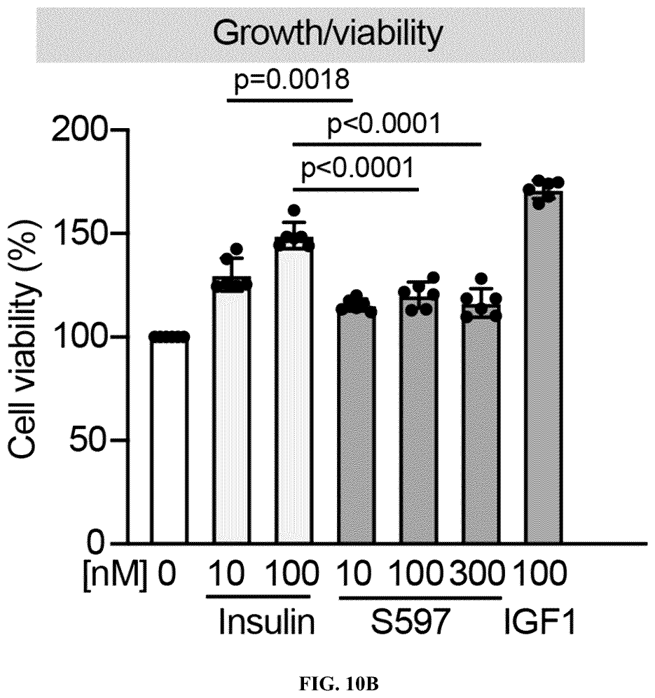

Insulin stimulates uptake of circulating glucose into cells, thus reducing blood glucose level. To investigate the metabolic effects of insulin mimetics in vivo, an insulin tolerance test (ITT) was performed using Humulin (human insulin) and mimetics (FIGS. 10A, 8F, 8G). As expected, administration of Humulin, S597, S597-S20D, and S597-ΔGly lowered blood glucose levels in mice. In contrast, administration of S597-N20 (S597-component-2 only), S597-W6A (S597-component-2 mutant), and S597-F27A (S597-component-1 mutant) did not reduce the blood glucose levels. These data validate the role of S597 in IR signaling, suggesting that S597 can overcome insulin resistance in patients with insulin-desensitized IR or even insulin binding-deficient IR mutants.

To examine the function of S597 on cell viability and growth, an MTT assay was performed in C2C12 cells. C2C12 cells were significantly increased in viability and growth by insulin and IGF1 but not by S597 (FIG. 10B), which suggests that S597 does not promote cell proliferation as efficiently as insulin, presumably due to the defect in MAPK pathways activation.

To investigate the metabolic effects of S597 and each component of S597 in vivo, insulin tolerance assays were performed in mice (FIGS. 12B-12D). Both S597 and S597-S20D lowered blood glucose in mice as effectively as insulin (FIGS. 12B, 12C), and S597-S20D displayed a longer lasting glucose lowering ability compared to S597 (FIG. 12D). By contrast, individual administration of S597-N20 (S597-component-2 alone), S597-W6A (S597-component-2 mutant), and S597-F27A (S597-component-1 mutant) did not reduce glucose levels, but rather elevated them, likely due to the partial binding of S597 mutants preventing basal insulin from binding and activating the IR. These data confirm that both components-1 and -2 of S597 are required for IR activation (FIGS. 12A, 12C). Taken together, these results further validate the function of S597 in IR signaling and show that S597 and a designed S597 analog (S597-S20D) can control metabolism as efficiently as insulin in mice.

### Example 6. Insulin and Insulin Mimetics Regulate Metabolism Differently In Vivo

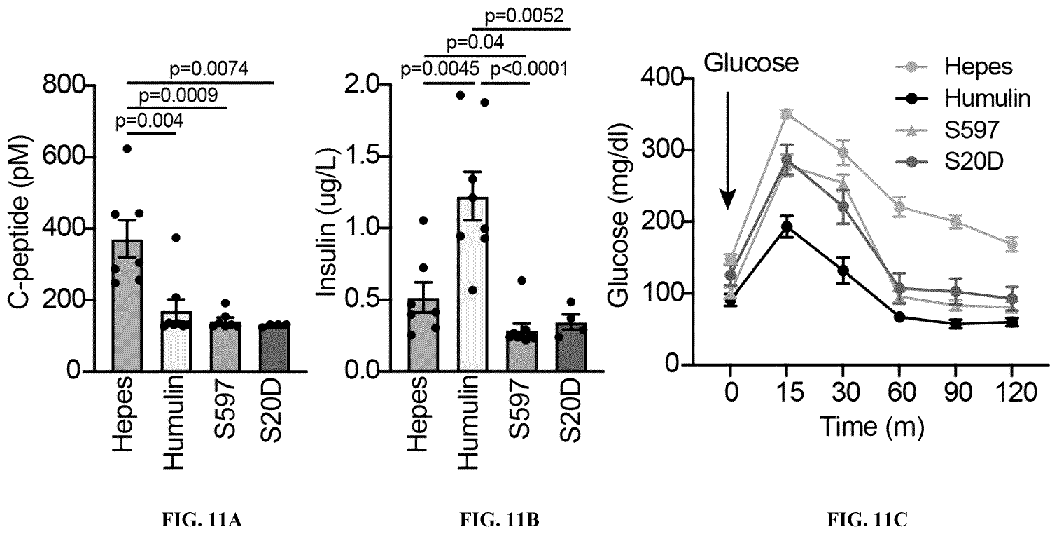

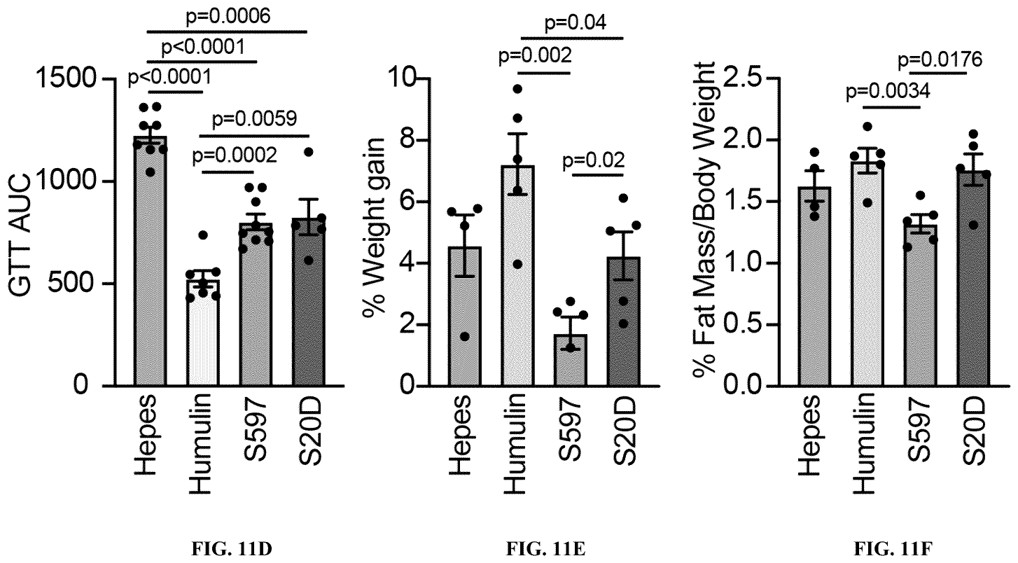

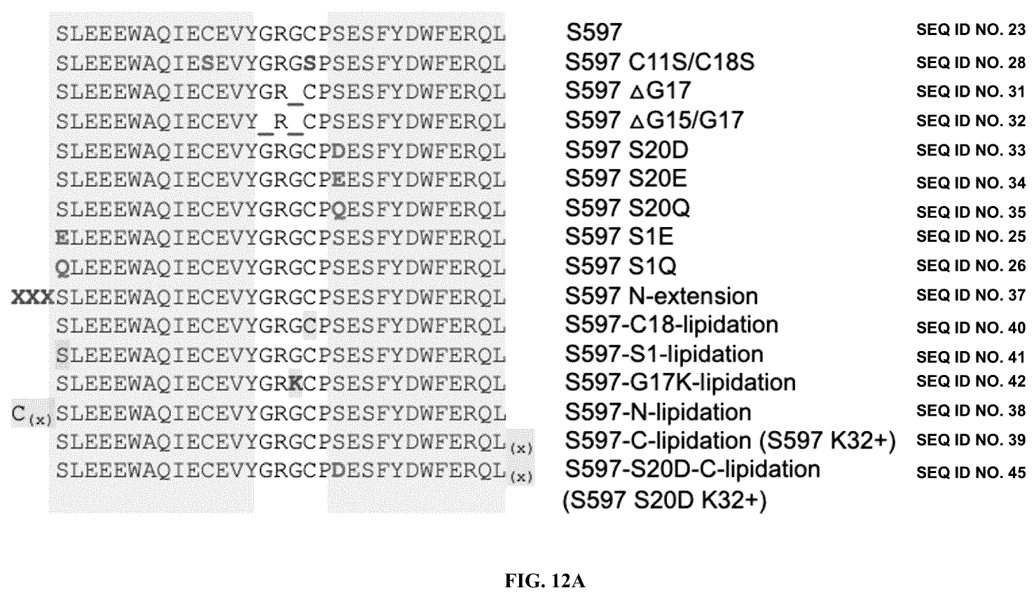

To examine the long-term effects of S597 in vivo, C57BL/6J WT mice were implanted with osmotic minipumps releasing either HEPES, Humulin, or mimetics. Chronic treatment of Humulin and mimetics decreased C-peptide (insulin secretion) (FIG. 11A). Insulin mimetics reduced circulating insulin levels (FIG. 11B), but improved glucose tolerance (FIGS. 11C, 11D). Consistent with previous work, chronic treatment of Humulin increased body weight (FIGS. 11E, 11F). However, S597-treated mice gained less than control mice. S597-S20D-treated mice gained similar weight to control mice. These data suggest that S597 has a different intrinsic ability to regulate metabolism in vivo from insulin, and this ability can be modulated by modest sequence changes.

### Example 7. Hyperinsulinemia and Cancer

Hyperinsulinemia is highly associated with the high risk of certain cancer types such as breast cancer, colon cancer, and liver cancer. It is common for patients to have both cancer and diabetes. Since insulin activates insulin receptor (IR) signaling and promotes cancer cell progression, treatment of hyperinsulinemia and/or insulin resistance in cancer patients has been a major challenge. Thus, whether S597 can activate IR signaling and proliferation in cancer cells was tested.

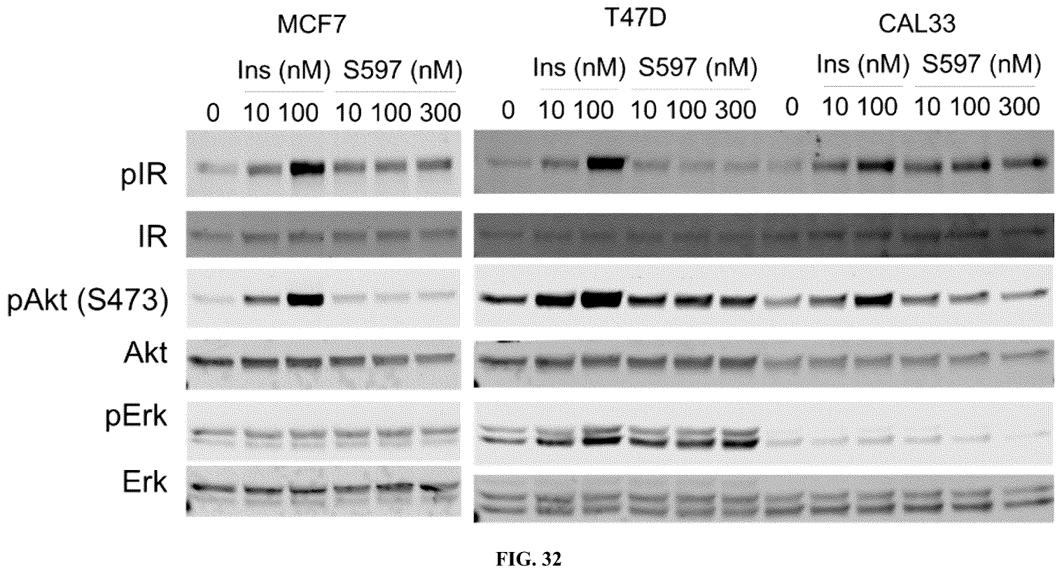

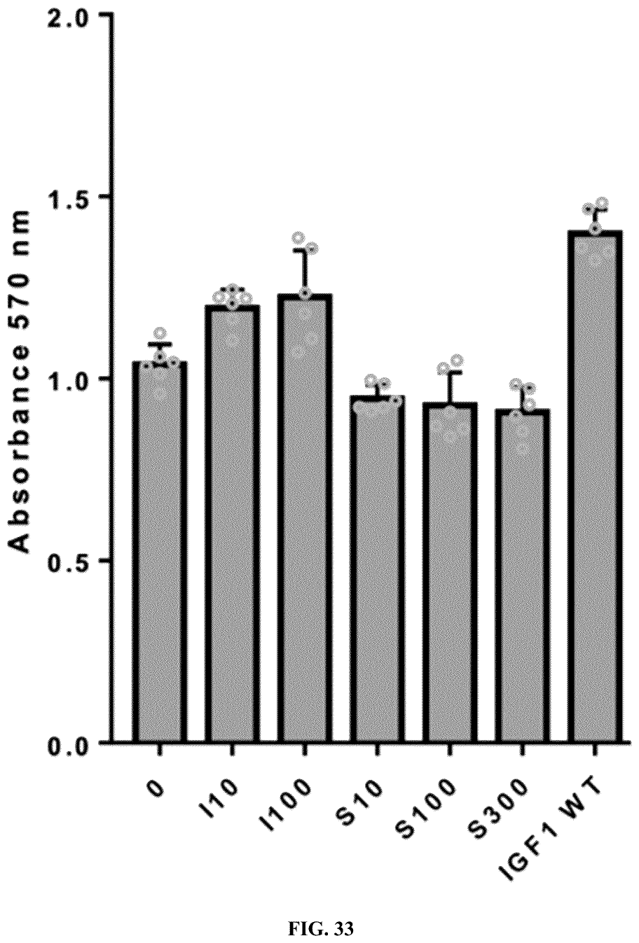

Despite S597 activating IR signaling in non-cancer cells, it failed to activate IR signaling in cancer cells including MCF7, T47D, and CAL33 (FIG. 32). Consistent with this, S597 did not promote cell proliferation in MCF7, T47D, and CAL33 (FIG. 33). S597 and the disclosed synthetic peptides would be beneficial patients with both cancer and diabetes to control glucose without promoting cancer cell progression.

### Example 8. Methods

a. Mice

Animal work described in this disclosure was approved and conducted under the oversight of the Columbia University Institutional Animal Care and Use Committee. Mice (C57BL/6J, stock #000664, Jackson laboratory) were fed a standard rodent chow (Lab diet, #5053). All animals were maintained in a specific antigen-free barrier facility (temperature, 68-79° F.; humidity, 30-70%) with 12 hr light/dark cycles (6 a.m. on and 6 p.m. off). Two to three-month old male mice were used in this study.

b. Cell Lines

FreeStyle™ 293-F cells were obtained from Invitrogen (R79007). FreeStyle™ 293-F cells were cultured in FreeStyle™ 293 Expression Medium. FreeStyle™ 293-F cells were maintained in orbital shaker in 37° C. incubator with a humidified atmosphere of 5% CO₂.

293 FT cells were obtained from Invitrogen (R70007). 293 FT cells were cultured in high-glucose Dulbecco's modified Eagle's medium (DMEM) supplemented with 10% (v/v) fetal bovine serum (FBS), 2 mM L-glutamine, and 1% penicillin/streptomycin. 293 FT cells were maintained in monolayer culture at 37° C. and 5% CO₂ incubator.

Spdoptera frugiperda (Sf9) cells were cultured in SF900 II SFM (Gibco) at 27° C. with orbital shaking at 120 rpm.

c. Cell Line Validation

An aliquot of each cell line was passaged for only 3-4 weeks, after which a fresh batch of cells was thawed and propagated. No mycoplasma contaminations were detected.

d. Synthesis of Insulin Mimetic Peptides

General reagents and methods. All standard solvents and reagents were obtained from Sigma-Aldrich unless otherwise specified, Fmoc-protected amino acid were purchased from Vivitide, H-Rink-ChemMatrix resin (loading level: 0.47 mmoml/g) from Gyros Technologies, trifluoroacetic acid (TFA) from EMD Millipore, piperidine from Alfa-Aesar, 1-hydroxy-6-chloro-benzotriazole (6-Cl—HOBt) from Creosalus-Advanced Chemtech, tri-isopropylsilane (TIS) from TCI. Analytical chromatography was performed on an Agilent 1100 model system equipped with an auto sampler using Phenomenex columns as specified below. Preparative chromatography was conducted on a Waters Model 2525 binary pump system equipped with a Model 2487 absorbance detector. Mass spectral data was collected on a Waters Synapt G2 HDMS.

Peptide Synthesis. The insulin mimetic sequences were assembled on 0.1 mmol scale by automated solid-phase methodology using an Applied Biosystems 431A synthesizer on H-Rink-ChemMatrix support. The standard α-fluorenyl-methoxycarbonyl/t-butyl (Fmoc/tBu) based protecting group scheme was used: Arg(Pbf), Asp(OtBu), Cys(Trt), Glu(OtBu), Gln(Trt), Ser(tBu), Trp(Boc), Tyr(tBu). The automated cycles utilized 6-Cl-hydroxybenztriazole (6-Cl—HOBt)/diisopropylcarbodiimide (DIC) for activation and 20% piperidine/DMF for deprotection. N-terminal acetylation was performed manually by treating the peptide resin with a DMF solution containing 5% acetic anhydride for 30 minutes. Resin cleavage and side-chain deprotection were carried in 15 ml of TFA containing 2% v/v of the following scavengers: water, TIS, thioanisole, β-mercaptoethanol for 2.0 hours. The crude peptide was recovered by addition of excess cold diethyl ether, followed by several washes of the precipitate with diethyl ether and air drying.

Oxidation/folding. Oxidation to form the disulfide bridge involved dissolving the crude peptide in a 25 mM ammonium bicarbonate buffer (pH 7.7) at a concentration of 1.0 mg/ml and stirring the solution in an open beaker at room temperature. The progress of the oxidation was monitored by analytical HPLC chromatography using a Phenomenex Gemini® 5 μm C18 150×4.6 mm analytical column and a linear gradient of 10-40% B (0.1% TFA/ACN) over 30 minutes. The oxidation was complete after 24-36 hrs. and the peptide isolated by preparative HPLC chromatography using a Phenomenex Luna® 10 μm C8(2) 250×21.2 mm LC column and a 15-30% B gradient over 60 mins. The homogeneity of the purified peptides was assessed by analytical HPLC and Mass Spectrometry to confirm product identity.

e. Synthesis of Insulin Receptor and its Mutant Forms

Protein expression and purification were performed following previous protocols. Briefly, the cDNA fragment of the short isoform of full-length mouse IR (MmIR) with kinase-dead mutation (D1122N) and IR substrates binding and endocytosis defective mutation (Y962F) was cloned into the pEZT-BM expression vector. It is noteworthy to note that the ectodomains of human and mouse IR have a sequence homology of ˜95.1% (FIG. 26). The HRV-3C protease recognition sequence and the protein Tsi3, which was used as purification tag, were fused to the C-terminus. MmIR was expressed in FreeStyle™ 293-F (Invitrogen, #R79007) cells using the BacMam system following the standard protocol. Protein was expressed in FreeStyle™ 293-F cells by infecting the virus at 1:10 (v:v) ratio. 6 hr. after infection, 8 mM sodium butyrate was added to boost protein expression. The cells were cultured for 48-60 hr. at 30° C.

The cells were resuspended in lysis buffer A containing 40 mM Tris-HCl pH 8.0, 400 mM NaCl and protease inhibitor cocktail (Roche). The membrane fraction was obtained by ultracentrifuge the cell lysate for 1 hr. at 100,000 g. 1% Dodecylmaltoside (DDM) was added with stirring to extract the MmIR from the membrane fraction. The solubilized protein was obtained by ultracentrifuge again for 1 hr. at 100,000 g. The supernatant was added with 1 mM CaCl2 and loaded to Tse3 protein conjugated Sepharose resin (GE Healthcare) by gravity flow. The resin was washed by wash buffer A (40 mM Tris-HCl, pH 8.0, 400 mM NaCl, 1 mM CaCl2, 5% glycerol, 0.08% DDM) and MmIR was eluted by HRV-3C protease cleavage. The protein was then run on Superose 6 increase 10/300 GL size-exclusion column (GE Healthcare) with buffer 20 mM Hepes-Na pH 7.4, 150 mM NaCl, 0.03% DDM. The dimer fraction was pooled and added with synthesized S597-component-2 or S597 at 1:4 (m: m). After incubation for half an hour, the protein was concentrated to 6 mg/ml for cryo-EM analyses. All the purification steps were performed at 4° C.

f. EM Data Acquisition

EM data acquisition, image processing, model building, and refinement were performed following previous protocols.

The cryo-EM grid was prepared by applying 3 μl of the protein samples (6 mg/ml) to glow-discharged Quantifoil R1.2/1.3 300-mesh gold holey carbon grids (Quantifoil, Micro Tools GmbH, Germany). Grids were blotted for 4.0 seconds under 100% humidity at 4° C. before being plunged into the liquid ethane using a Mark IV Vitrobot (FEI). Micrographs were acquired on a Titan Krios microscope (FEI) operated at 300 kV with a K3 direct electron detector (Gatan), using a slit width of 20 eV on a GIF-Quantum energy filter. SerialEM 3.8 was used for the data collection. A calibrated magnification of 46,296 was used for imaging of IR/S597-component-2 sample, yielding a pixel size of 1.08 Å on specimen. A calibrated magnification of 60,241 was used for imaging of IR/S597 sample, yielding a pixel size of 0.83 Å on specimen. The defocus range was set from 1.6 μm to 2.6 μm. Each micrograph was dose-fractionated to 30 frames with a total dose of about 60 e⁻/Å².

g. Image Processing

The cryo-EM refinement statistics for both IR/S597-component-2 and IR/S597 datasets are summarized in Table 1. 8,388 movie frames of IR/S597-component-2 were motion-corrected and binned two-fold, resulting in a pixel size of 1.08 Å, and dose-weighted using MotionCor2. The CTF parameters were estimated using Gctf1.06. RELION3 was used for the following processing. Particles were first roughly picked by using the Laplacian-of-Gaussian blob method, and then subjected to 2D classification. Class averages representing projections of IR/S597-component-2 in different orientations were used as templates for reference-based particle picking. Extracted particles were binned three times and subjected to 2D classification. Particles from the classes with fine structural feature were selected for 3D classification using an initial model generated from a subset of the particles in RELION3. Particles from one of the resulting 3D classes showing good secondary structural features were selected and re-extracted into the original pixel size of 1.08 Å. Subsequently, finer 3D classification were performed with C2 symmetry imposed by using local search in combination with small angular sampling, resulting in two new good classes with improved density for the entire complex. The cryo-EM map after 3D refinement of the combined two classes was resolved at 3.6 Å resolution, but the stalk region of the complex appeared blurred, suggesting relative swinging motions between two halves of the complexes. To improve the resolution, C2 symmetry expansion and focused refinement were performed as described previously. The modified particle set was subjected to another round of 3D refinement with a soft mask around one half of the complex, leading to a markedly improved resolution for the entire half complex to 3.5 Å resolution.

TABLE 1
Cryo-EM Data Collection and Refinement Statistics

| | IR/S597-N20 | IR/S597 |
|---|---|---|
| | EMD-27705; | EMD-27704; |
| | PDB: 8DTM | PDB: 8DTL |
| Data collection and processing | | |
| Magnification | 46,296 | 60,241 |
| Voltage (kV) | 300 | 300 |
| Electron exposure (e⁻/Å²) | 60 | 60 |
| Defocus range (μm) | 1.6-2.6 | 1.6-2.6 |
| Pixel size (Å) | 1.08 | 0.83 |
| Symmetry imposed | C1 | C2 |
| Initial particle images (no.) | 2,789,169 | 1,282,550 |
| Final particle images (no.) | 135,709 | 152,565 |
| Map resolution (Å) | 3.5 | 5.4 |
| FSC threshold | 0.143 | 0.143 |
| Refinement | | |
| Initial model used (PDB code) | 4ZXB | 6PXV |
| Model composition | | |
| Nonhydrogen atoms | 6,500 | 12,518 |
| Protein residues | 802 | 1,544 |
| Ligands | | |
| R.m.s. deviations | | |
| Bond lengths (Å) | 0.009 | 0.003 |
| Bond angles (°) | 1.098 | 0.506 |
| Validation | | |
| MolProbity score | 1.99 | 1.82 |
| Clashscore | 10.23 | 9.6 |
| Poor rotamers (%) | 0 | 0 |
| Ramachandran plot | | |
| Favored (%) | 92.82 | 95.47 |
| Allowed (%) | 7.18 | 4.53 |
| Disallowed (%) | 0 | 0 |

4,615 movie frames of IR/S597 were motion-corrected and binned two-fold, resulting in a pixel size of 0.83 Å, and dose-weighted using MotionCor2. CTF correction was performed using Gctf. Particles were first roughly picked by using the Laplacian-of-Gaussian blob method, and then subjected to 2D classification. Class averages representing projections of the IR/S597 in different orientations were used as templates for reference-based particle picking. A total of 1,282,550 particles were picked from 4,615 micrographs. Particles were extracted and binned three times (leading to 2.49 Å/pixel) and subjected to another round of 2D classification. Particles in good 2D classes were chosen (616,622 in total) for 3D classification using an initial model generated from a subset of the particles in RELION3. After initial 3D classification, one major class was identified showing good secondary structural features. Particles from this good class were selected and re-extracted into the original pixel size of 0.83 Å. Subsequently, finer 3D classification with C2 symmetry imposed by using local search were performed in combination with small angular sampling, resulting one 5 good classes with similar conformation and 1 bad class with poor density throughout the entire IR/S597 complex. Five good classes were combined, and the cryo-EM map after 3D refinement, CTF refinement and particles polishing was resolved at 5.4 Å resolution.

h. Model Building and Refinement

Model building of IR/S597-component-2 was initiated by rigid-body docking of the apo-state of apo-IR with minor adjustment. The model of S597-component-2 was de novo built using Coot0.8.8. Model building of IR/S597 was initiated by rigid-body docking of the structures of S597-component-1 bound L1, CR, L2, and S597-component-2 bound FnIII1, FnIII-2 and FnIII-3 domains. Both models were refined by using the real-space refinement module in the Phenix package (V1.17). Restraints on secondary structure, backbone Ramachandran angels, residue sidechain rotamers were used during the refinement to improve the geometry of the model. MolProbity 4.5 as a part of the Phenix validation tools was used for model validation (Table 1). Figures were generated in Chimera 1.15 and Pymol 2.3.

i. Primary Hepatocytes Isolation

Primary hepatocytes were isolated from 2-month-old male mice with a standard two-step collagenase perfusion procedure as described earlier with some modifications. Briefly, following anesthesia, the inferior vena cava was cannulated, and the liver was perfused with Liver Perfusion Medium (Thermo Scientific, Cat. #17701038) using peristaltic pump (perfusion rate as 3 ml/min). After 1-2 sec upon appearance of white spots in the liver, the portal vein was cut with scissors to wash out blood, and then the liver was perfused with 30 ml of Liver Digest Medium (Thermo Scientific, Cat. #17703034). Dissected liver was gently washed with low glucose DMEM and transferred to sterile culture dish containing 15 ml Liver Digest Medium. The isolated liver cells were filtered through the 70 μm cell strainer into a 50 ml tube. After centrifuge at 50 g for 5 min at 4° C., cells were washed with cold low glucose DMEM three times. Cells were resuspended with attached medium [Williams' Medium E supplemented with 5% (v/v) FBS, 10 nM insulin, 10 nM dexamethasone, and 1% penicillin/streptomycin] and plated on collagen (Sigma, #C3867)-coated dishes. After 2 hr., the medium was changed to serum free low-glucose DMEM. After 14-16 hr., the cells were treated with insulin, S597 or S597-N20 to analyze IR signaling.

j. Mouse Embryonic Fibroblast (MEFs) Isolation and Viral Infection

IR^{F/F} mice (Jackson #006955) were crossed with CAG-CRE/ERT mice (Jackson #004682) to generate IR^{F/F}; CRE-ERT mice. The IR^{F/F}; CRE-ERT mice were backcrossed 9 generations to C57BL/6 (Jackson #000664). The MEFs from IR^{F/F}; CRE-ERT mice were isolated as described earlier with some modifications. E14.5 embryos were minced with sterile scalpels and incubated with 0.25% trypsin-EDTA for 10 min at 37° C. and 5% CO₂ incubator. Fresh 0.25% trypsin-EDTA was added. After 10 min incubation, cells were put into fresh DMEM containing supplemented 10% (v/v) FBS, 2 mM L-glutamine, 55 μM β-mercaptoethanol, and 1% penicillin/streptomycin, and then centrifuged at 290 g for 5 min. After the supernatant was removed, the cells were resuspended in the DMEM and incubated at 37° C. and 5% CO₂ incubator. The medium was changed the next day to remove debris and floating cells.

To generate IR-D707A MEFs or GFP MEFs, cDNAs encoding C-terminal GFP-tagged human IR D707A or GFP alone were cloned into the pBabe-puro vector. pBabe-puro-IR D707 Å-GFP or pBabe-puro-GFP, pCMV-gag/pol, and pCMV-VSV-G were transfected into 293 FT cells using Lipofectamine™ 2000 (Invitrogen). Viral supernatants were collected at 2 days and 3 days after transfection and concentrated with concentrator and added to IR^{F/F}; CRE-ERT MEFs with 4 μg/ml of polybrene. Cells were selected with 2 μg/ml of puromycin at 2 days after infection.

k. Insulin Receptor Activation and Signaling Assay in Cultured Cells

The insulin receptor signaling assay was performed as described earlier with some modifications. For the activation assay, the short isoform of human IR in pCS2-MYC was used as described previously. Plasmid transfections into 293 FT cells were performed with Lipofectamin™ 2000 (Invitrogen). One day later, the cells were serum starved for 14 hr. and treated with the indicated concentrations of insulin or S597 analogs. For IR-D707A MEFs, cells were incubated with 1 μM Tamoxifen (EMD Millipore, Cat. #508225) for 2 days, starved for 14 hr., and treated with the insulin or S597 analogs.

The cells were incubated with the lysis buffer B [50 mM Hepes pH 7.4, 150 mM NaCl, 10% Glycerol, 1% Triton X-100, 1 mM EDTA, 100 mM sodium fluoride, 2 mM sodium orthovanadate, 20 mM sodium pyrophosphate, 0.5 mM dithiothreitol (DTT), 2 mM phenylmethylsulfonyl fluoride (PMSF)] supplemented with complete Protease Inhibitor Cocktail (Roche), PhosSTOP (Sigma), and 25 U/ml turbo nuclease (Accelagen) on ice for 1 hr. After centrifuge at 20,817 g at 4° C. for 10 min, the concentrations of cell lysate were measured using Micro BCA Protein Assay Kit (Thermo Scientific). About 50-60 μg total proteins were analyzed by SDS-PAGE and quantitative Western blotting. The following antibodies were purchased from commercial sources: Anti-IR-pY1150/1151 (WB, 1:1000; 19H6; labeled as pY IR, Cat. #3024), anti-AKT (WB, 1:1000; 40D4, Cat. #2920), anti-pS473 AKT (WB, 1:1000; D9E, Cat. #4060), anti-ERK1/2 (WB, 1:1000; L34F12, Cat. #4696), anti-pERK1/2 (WB, 1:000; 197G2, Cat. #4377), anti-IRS1-pS616 (WB, 1:1000; C15H5, Cell Signaling, Cat. #3203); anti-IRS1 (WB, 1:1000; A301-158A, Bethyl Laboratory); anti-IR (WB, 1:500, CT-3, Santa Cruz; labeled as IR for primary hepatocytes and MEFs, Cat. #sc-57342); anti-IR (WB, 1:1000; EPR23566-103; labeled as IR for FIG. 31, ab278100, Abcam); anti-Myc (WB, 1:2000; 9E10, Cat. #11667149001, Roche; labeled as IR for 293 FT cells). For quantitative western blots, anti-rabbit immunoglobulin G (IgG) (H+L) (WB, 1:5000; Dylight 800 conjugates, Cat. #5151) and anti-mouse IgG (H+L) (WB, 1:5000; Dylight 680 conjugates, Cat. #5470) (Cell Signaling) were used as secondary antibodies. The membranes were scanned with the Odyssey Infrared Imaging System (Li—COR, Lincoln, NE).

l. Insulin Signaling and Insulin Receptor Endocytosis In Vivo

2-3 months old male mice were fasted overnight. Following anesthesia, mice were injected with 6 nmol Humulin (Eli Lilly) or 9 nmol S597 analogs per mouse via inferior vena cava. Livers were removed at 3 min after injection. White adipose tissue and skeletal muscles were removed at 5 min and 7 min after injection, respectively. Tissues were mixed with the lysis buffer B supplemented with complete Protease Inhibitor Cocktail (Roche), PhosSTOP (Sigma), and 25 U/ml turbo nuclease (Accelagen), homogenized with Fisherbrand™ Bead Mill homogenizer, and then incubated on ice for 1 hr. After centrifuge at 20,817 g at 4° C. for 30 min, the concentrations of cell lysate were measured using Micro BCA Protein Assay Kit (Thermo Scientific). The lysates were then analyzed by quantitative western blotting.

For IR endocytosis assays, the livers were fixed in 10% Neutral Buffered Formalin (NBF) and embedded in paraffin blocks. Sections were deparaffinized, subjected to immunohistochemistry as described in the immunohistochemistry section and previous study.

m. Immunohistochemistry

The immunohistochemistry was performed as described earlier with some modifications. Mouse tissues were fixed in 10% NBF and embedded in paraffin blocks by the Molecular Pathology Core at Columbia University. The deparaffinized sections were subjected to antigen retrieval with 10 mM sodium citrate (pH 6.0), incubated with 0.3% H2O2, blocked with 3% bovine serum albumin (BSA), and then incubated with anti-IR antibodies (CT-3, Millipore, Cat. #MABS65, 1:100) in 3% BSA in PBS with 0.1% Triton X-100 (0.1% PBST) at 4° C. overnight. The slides were washed and incubated with secondary antibodies (Alexa Fluor 568 donkey anti-mouse, Invitrogen, Cat. #A10037, 1:200). The washed slides were stained with 4′,6′-diamidino-2-phenylindole (DAPI). Ten images were randomly taken under x60 magnification with a Leica Thunder Imager (Leica Microsystems Inc.). Images of sections were acquired as series of 0.2 μm stacks. The cell edges were defined with Image J as described earlier. The whole cell signal intensity (WC) and intracell cellular intensity (IC) were measured. The plasma membrane signal intensity (PM) was calculated by subtracting IC from WC. Identical exposure times and magnifications were used for all comparative analyses.

n. Immunofluorescence Assay for SREBP1 Translocation

Primary hepatocytes were as described in the primary hepatocyte isolation section. To analyze SREBP1 translocation, the isolated hepatocytes were resuspended with modified attached medium [Williams' Medium E supplemented with 10% (v/v) FBS, 100 pM insulin, 2 mM L-glutamine, 100 nM dexamethasone, 5.5 μg/mL transferrin, 6.7 ng/mL sodium selenite, and 1% penicillin/streptomycin] and plated on collagen-coated coverslips. After 14 hr., cells were pre-treated with 10 μM MG-132 (MedChemExpress LLC, Cat. #HY-13259) for 2 hr., and then treated with 100 nM insulin (Sigma) or S597 for 30 min. Cells were fixed with 4% paraformaldehyde for 10 min. The fixed cells were incubated with PBS for 30 min and 3% BSA in PBS with 0.1% Triton X-100 (0.1% PBST) for 1 hr., and then treated with anti-SREBP1 antibodies (20B12, Millipore, Cat. #MABS1987, 1:200) in 3% BSA in 0.1% PBST at 4° C. overnight. After being washed, cells were incubated with secondary antibodies (Alexa Fluor 488 goat anti-rabbit, Invitrogen, Cat. #A11008, 1:200) for 1 hr. Cells were washed and mounted on microscope slides in ProLong Gold Antifade reagent with DAPI (Invitrogen). Images were acquired as a series of 0.2 μm stacks with a Leica Thunder Imager (Leica Microsystems Inc.). Raw images were deconvolved using LAS X Software (Leica Microsystems Inc.). The nuclear edges and the nuclear SREBP1 signal were defined with Image J. Identical exposure times and magnifications were used for all comparative analyses.

o. Insulin Tolerance Test

Insulin tolerance tests were performed as described previously with some modification. Briefly, 2-3 months old male mice were fasted for 2 hr. and their blood glucose levels (T=0) were measured with tail bleeding using a glucometer (AlphaTRAK). After then, mice were injected intraperitoneally with Humulin (6 nmol/kg body weight), S597 (9 nmol/kg), S597-S20D (9 nmol/kg), S597-W6A (9 nmol/kg), S597-F27A (9 nmol/kg), or S597-N20 (27 nmol/kg), and their blood glucose levels were measured at the indicated time points after injection.

p. Chronic S597 Treatment and Glucose Tolerance Test

For FIGS. 31A-31E, 2 months old male mice were injected intraperitoneally with vehicle, insulin (50 U/kg body weight) or S597 (50 U/kg body weight) once daily for three days. For oral glucose tolerance test, mice were fasted for 6 hr. and their blood glucose levels (T=0) were measured with tail bleeding using a glucometer (AlphaTRAK). After then, glucose (2 g/kg body weight) was administered by oral gavage. Blood glucose levels were measured at the indicated time points after glucose administration. For FIG. 31F, 2 months old male mice were fasted for 14 hr. Following anesthesia, mice were injected with vehicle (PBS), insulin (0.1 U/kg), S597 (0.15 U/kg), or S597-S20D (0.15 U/kg) via jugular vein. One hour later livers were collected.

q. Statistical Analysis

Prism 9 was used for the generation of graphs and for statistical analyses. Results are presented as Mean±SD or Mean±SEM. Two-tailed unpaired t tests were used for pairwise significance analysis. Two-way ANOVA followed by Tukey's multiple comparisons test was used for insulin tolerance test analysis. Sample sizes were determined on the basis of the maximum number of mice. Power analysis for sample sizes was not performed. Randomization and blinding methods were not used, and data were analyzed after the completion of all data collection in each experiment.

r. Data Availability

All reagents generated in this study are available with a completed Materials Transfer Agreement. All cryo-EM maps and models reported in this work has been deposited into EMDB/PDB database, under the entry ID: EMD-27704 (IR/S597), PDB 8DTL [https://www.rcsb.org/structure/unreleased/8DTL] (IR/S597), EMD-27705 (IR/S597-N20), and PDB 8DTM [https://www.rcsb.org/structure/unreleased/8DTM] (IR/S597-N20). PDB used in this study are as follows: 4ZXB [https://www.rcsb.org/structure/4ZXB] and 6PXV [https://www.rcsb.org/structure/6PXV]. Source data are provided with this paper.

### Example 9. Future Methods for Determining the Mechanism and Physiological Action of Insulin Mimetics

a. Function of Insulin Mimetics in IR Signaling

In vitro insulin mimetics activity: The effect of insulin mimetics and IR mutants on glucose uptake and lipolysis in DKO-IR, glucose production and lipogenesis in hepatocytes, and glucose uptake and glycogen synthesis in C2C12-IR will be analyzed. To examine the function of insulin mimetics in cell proliferation and viability, MTT assays will be performed using mimetics as shown in FIG. 10B.

Insulin mimetics binding assay: To directly measure the binding affinity and kinetics of S597-S20D and -ΔGly to IR (FIG. 19), previously employed in vitro insulin competition-binding assay and isothermal titration calorimetry will be performed using isolated full-length IR-WT, L1-CR-L2/αCT (site-1), and FnIII-1 (site-2) domains as shown in FIG. 9C.

b. IR Signaling and IR Complexes Induced by Insulin and Mimetics

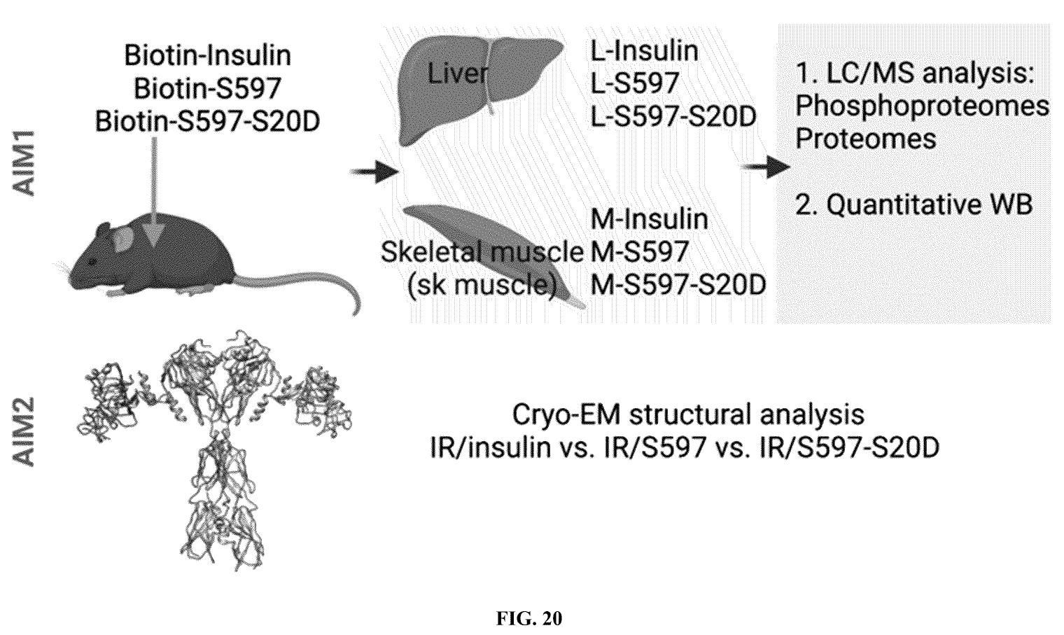

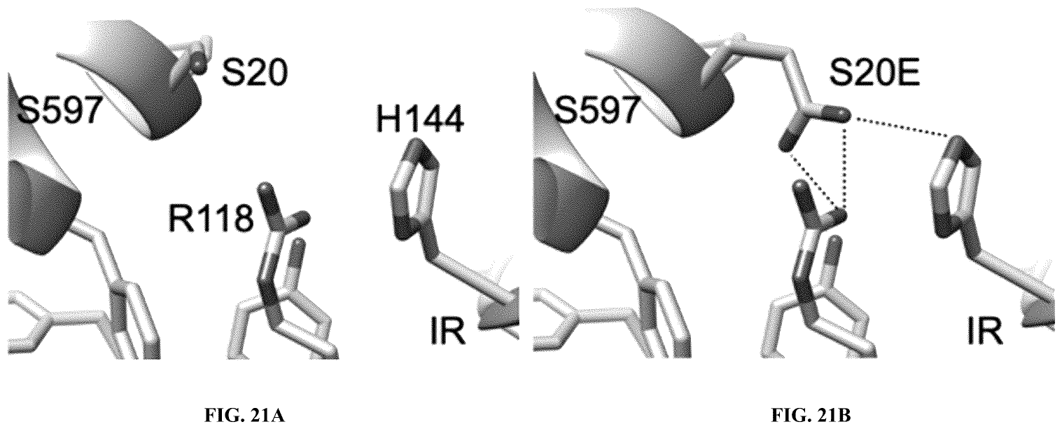

It is also possible that S597 affects the kinetics of IR internalization and signaling inside the cells. The preliminary data suggest that a modest change of S597 modulates its agonist action (FIGS. 6A-6C, 11A-11F). Through a continued collaboration with the IDeA National Resource for Quantitative Proteomics, quantitative phosphoproteomics of skeletal muscle and liver after biotin-insulin, or biotin-mimetics injection using multiplex tandem mass tag (TMT) labelling coupled with liquid chromatography/mass spectrometry (LC/MS) (FIG. 20) will be performed. Phosphoproteomics will show specific phosphopeptides that significantly altered in mimetics-treated samples and identify S597-sensitive phosphorylation residues and proteins as shown in FIG. 6C. Phospho-deficient or -mimicking mutants will be generated and their ability to complement cells depleted of the relevant proteins in IR signaling will be tested. Phospho-specific antibodies against key functional sites will be made and these phosphorylation events will be confirmed to be regulated by mimetics. Based on the mean abundance of each of these phosphopeptides, unsupervised cluster analysis may group skeletal muscle-insulin samples with sk muscle-S597-S20D samples, whereas skeletal muscle-S597 samples may not. As the pAKT and pERK levels in the S597-treated liver were similar to those in insulin-treated liver, the phosphorylation by S597 in the liver may be similar to those by insulin, compared to the differences in the muscle.

Additionally, quantitative proteomic studies of skeletal muscle and liver of the mice will be performed using TMT labelling coupled with LC/MS. Insulin or S597 analogs complexes as developed previously will be purified with some modifications. Briefly, after membrane solubilization and centrifugation of the biotin-ligand stimulated tissue samples, the supernatants will be incubated with streptavidin beads. The washed beads will be eluted and then analyzed by LC/MS and quantitative WB. IR/insulin or IR/mimetics complexes in the liver and skeletal muscle of mice (FIG. 20) will be compared. By comparison, the following may be identified: (1) protein(s) that specifically interacts with S597-bound IR in different tissues or (2) protein(s) associated with more insulin-bound IR or S597-S20D-bound IR. If such proteins exist, whether knockdown or overexpression of these proteins change IR signaling in primary hepatocytes and L6 cells will be tested. The insulin, S597, and S597-S20D complexes will be compared using quantitative immunoprecipitation-WB analysis with antibodies against specific proteins to confirm the complexes change. Whether these proteins directly bind to IR using an in vitro binding assay will be tested. If so, the binding sites of IR will be mapped, the binding surface will be mutated, and the IR signaling and trafficking in hepatocytes and L6 cells will be examined. These experiments will show a global change of phosphorylation of proteins as well as IR interacting proteins by S597, providing the molecular mechanism of biased signaling.

c. Physiological Function of Insulin Mimetics

S597 reduces blood glucose level, mimicking the action of insulin (FIGS. 8A-10B). Interestingly, S597 lowers blood triglyceride (TG) and hepatic de novo synthesis compared with insulin and prevents diabetes-associated atherosclerosis. Mice fed a HFD (60% kcal from fat) had impaired glucose tolerance and other type 2 diabetes phenotypes. The metabolic effects of insulin mimetics will be analyzed by injecting newly developed insulin mimetics in mice fed normal chow and HFD for 4 weeks. Insulin and S597 will be used as controls. In parallel, tissues samples (e.g., liver, sk muscle, and WAT) will be taken from mice treated with or without insulin mimetics and perform quantitative RT-PCR for insulin-mediated gene expression (e.g., lipogenic target genes, Fasn, Scd1, Srebp1c, etc.; glucose metabolism, Pepck, Gck, G6pc, etc.), quantitative western blot (WB) analysis for IR signaling, and immunohistochemistry for IR cellular localization.

High-fat diet model: S597 reduces glucose level, mimicking the action of insulin (FIG. 10). Interestingly, S597 lowers blood triglyceride (TG) and hepatic lipogenesis compared with insulin and prevents diabetes-associated atherosclerosis. The preliminary data showed that S597 reduces weight gain and fat mass relative to insulin, and S597-S20D moderates the S597-mediated reduction (FIG. 11). A HFD (60% kcal from fat) impairs glucose tolerance and other type 2 diabetes phenotypes, as well as altering lipid dynamics in mice. To validate this, C57BL/6J mice will be fed a HFD for 3 weeks and then surgically implanted with an osmotic pump releasing Hepes (control), Humulin, or mimetics (i.e., S597, -S20D, -ΔGly). In addition, to investigate the effects of chronic insulin mimetics on animal physiology and to examine unwanted immune responses (immunogenicity), the metabolic effects of insulin mimetics will be analyzed by implanting an osmotic pump releasing Hepes, Humulin or mimetics in mice fed normal chow. Body weight, biochemical indicators (e.g., glucose and HbA1c for glucose homeostasis; insulin and C-peptide for insulin homeostasis; free fatty acid, cholesterol and TG for lipid homeostasis; IgG etc.), and glucose and insulin sensitivity will be analyzed. Body mass and adiposity will first be normalized between groups. If mimetics-treated mice display resistance to diet-induced obesity, longitudinal feeding and pair-feeding studies will be performed to determine if food intake is reduced in mimetics-treated mice. If mimetics-treated mice cat a similar amount of food, this suggests that the mimetics differently control energy expenditure, body mass, and adiposity from insulin. If so, metabolic parameters such as body composition, energy expenditure, lipolysis, intramuscular TG/glycerol content, and body temperature will be analyzed. In parallel, tissues samples (e.g., liver, sk muscle, adipose tissues, kidney, pancreas, spleen, brain, etc.) will be taken from above mice, and quantitative RT-PCR for insulin-mediated gene expression (e.g., lipogenic target genes, Fasn, Scd1, Srebp1c, etc.; glucose metabolism, Pepck, Gck, G6pc, etc.), quantitative WB analysis for IR signaling, and immunohistochemistry for IR cellular localization will be performed. The adipose tissue mass will be determined using quantitative magnetic resonance before the implantation of the pump and at the end of the treatment period.

Streptozotocin (STZ)-induced model: Injection of multiple, sub-diabetogenic doses of STZ to mice causes pancreatic insulitis that progresses to near complete destruction of beta cells, mimicking type 1 diabetes phenotypes in mice. To avoid a potential interference of endogenous insulin, STZ-induced C57BL/6J mice (mice with non-fasting blood glucose cxceeding 250 mg/dL, Jackson laboratory) will be implanted with an osmotic pump releasing Humulin or insulin mimetics. Metabolic characteristics of mice will be analyzed for 3 weeks after implantation. The condition of the islet of Langerhans will be assessed with H&E staining.

Pharmacokinetic characteristics of insulin mimetics: To examine the activity of insulin mimetics in the body over a period of time, antibodies specific to S597 were generated. Insulin mimetics will be administered subcutaneously to C57BL/6J mice fed normal chow diet at doses of 9, 27, and 40 nmol/kg of body weight. Blood samples will be collected 15, 30, 45, 60, 90, 120, 240, and 480 min after injection. The levels of insulin mimetics will be determined by ELISA.

To investigate the effects of chronic insulin mimetics on animal physiology and to examine unwanted immune responses (immunogenicity), C57BL/6 WT mice (fed normal chow or HFD for 4 weeks) will be treated with twice-daily injection of vehicle, insulin (6 nmol/kg), or insulin mimetics (6 and 9 nmol/kg) for 3 months. Body weight and biochemical indicators (e.g., serum glucose, insulin, free fatty acid, cholesterol, TG, IgG etc.) will be analyzed and a histological analysis will be performed including liver, muscle, WAT, kidney, pancreas, spleen and brain.

d. Generate Mouse Models of Insulin Receptoropathy to Study Whether Insulin Mimetics Restore IR Functions

To elucidate whether insulin mimetics restores IR functions in vivo with insulin binding-deficient IR mutants, mouse models of insulin receptoropathy (IR-D709A, C57BL/6) were generated. IR mutation found in Donohue syndromes, D709A (D707A in human IR), was selected for study based on preliminary data in cell signaling assays. D709A mutation produces receptors that are normally processed and expressed at the cell surface but showed severely reduced insulin-mediated IR activation (FIG. 5). The cryo-EM structure confirmed that D709 is critical for insulin binding (FIGS. 1, 5A). Importantly, IR D707A could be activated by S597 (FIG. 5). IR^{D709A/+} mice will be crossed with tamoxifen-inducible IR knockout (IR^{F/F}; Cre-ERT) to generate IR^{F/D709A}; Cre-ERT. These mice express IR-WT until induced expression of Cre recombinase.

Determine the function of IR mutants and insulin mimetics (pharmacodynamic study). Tamoxifen will be administrated into mice at 4 weeks of age. The metabolic effects of IR mutants and insulin mimetics will be analyzed using glucose tolerance test and ITT. To demonstrate the pharmacodynamic similarity among insulin, S597, and S597-S20D, euglycemic clamp will be performed in WT and IR^{D709A/-} mice. Using radio-labeled glucose during clamp, glucose metabolism will be measured in individual organs including liver, WAT, and sk muscle. Hepatic glucose production, glucose uptake (WAT and sk muscle), and glycogen synthesis (liver and sk muscle) will be analyzed. The tissue samples will be taken and utilized for quantitative WB analysis for IR signaling, and immunohistochemistry or fractionation of IR cellular localization.

### Example 10. Future Methods for Elucidating the Cryo-EM Structures of IR Bound to Mimetics

a. Identifying Factors that are Specifically Bound with IR/S597 or IR/S597-S20D

The specific cytosolic adapter proteins that are recruited to IR activated by either S597 or S597-S20D will be identified. Briefly, MS-based proteomics will be applied to quantitatively compare the proteins that co-purify with S597- or S597-S20D-bound IR. Unliganded IR will be used as a negative control to filter out non-specific contaminants. In this way, the cytosolic proteins that can selectively bind to S597- or S597-S20D-activated IR will be identified. The cryo-EM structures of IR/S597 and IR/S597-S20D in complex with those ligand-biased adapter proteins will be solved. These structures will explain how S597- and S597-S20D-activated IR can preferentially recruit certain downstream effector proteins, leading to a biased signaling. The binding of these newly identified partner proteins will further rigidify IR conformation, facilitating the structural determination of the complete IR/S597 complex.

b. Understand the Mechanisms of the Biased Signaling Induced by S597

To understand the structural basis for the biased signaling induced by S597, the cryo-EM structures of IR/S597-S20D complex will be determined. Extensive 3D classification for each dataset will be performed to compare the conformational landscapes of IR induced by S597 and S597-S20D. 3D refinement for the major class from each dataset will be performed to obtain the high-resolution 3D reconstruction for detailed structural comparison.

In the case that TM and kinase domains are not resolved in the cryo-EM maps, the conformations of the membrane-proximal domains of IR induced by S597 and S597-S20D will be compared. Because the membrane-proximal domain is connected to the TM domain through a short linker (4 residues), it is hypothesized that extracellular, TM and intracellular domains are coupled allosterically, and that the differences in the arrangements of the membrane proximal regions in the IRs bound with S597 or S597-S20D may cause differential dimeric assembly of TM and intracellular domains. Subsequently, the differentially assembled kinase domains may lead to distinct phosphorylation patterns, which would allow the phosphorylated kinase to preferentially engage different types of downstream effector proteins. Such structural coupling could partially explain the functional results that different types of S597 analogs could trigger distinct downstream signaling. These structural and functional studies will provide insight into the structural and molecular basis of biased signaling. Because S597-S20D partially restores the MAPK activation in the skeletal muscle, this experiment will demonstrate the potential of structure-based insulin mimetics design that recapitulate true insulin-like signaling and hold promise for the treatment of insulin resistance disease.

One strategy for improving the cryo-EM map of dynamic IR/S597 complex to a resolution that allows the unambiguous de novo modelling is to collect a large amount of data and utilize extensive experience in image classification to find a homogenous subset of particles for 3D reconstruction. Recent developments in cryo-EM image processing will also be applied, including “multibody” refinement in RELION4 and non-uniform and 3D-flex refinement in cryoSPARC, to improve the local resolution and obtain the best possible maps for the flexible regions. Alternatively, the atomic resolution structure of IR bound with the components-1 and -2 of S597 and other analogs will be determined separately. The preliminary structural results showed that the S597-component-2 binds to the FnIII-1 domain of IR in an identical manner as that observed for the IR/S597 complex (FIGS. 4C, 4E). Likewise, the IR bound with only the S597-component-1 is likely to be more suitable for high resolution cryo-EM study. The structural information from the structures of IR/S597-component-1 and IR/S597-component-2 will be combined to construct a precise model for the entire IR/S597 complex in the active state. Additionally, a Titan Krios equipped with the latest Falcon4i DED and Selectris energy filter which can greatly improve SPA resolution is accessible. Due to the flexibility of TM and kinase domains, performing focused classification is critical in cryo-EM image processing. A developed focused classification method (masked classification with residual signal subtraction) will be applied to improve the cryo-EM density for the local flexible region of a large protein complex. The structures and functions of IR on the cell surface are regulated by many molecules in the environment, including lipids and carbohydrates. Some of these may contribute to the conformational stability of the receptor. However, in traditional approaches, purified receptors from recombinant sources lack these cellular factors. Thus, IR that are purified directly from a native source (e.g., liver or placenta) will be studied by cryo-EM.

Directly visualizing the TMs and kinase in the dimerized functional form will be an important step toward fully revealing the structural differences of IRs induced by S597 and other analogs. To improve the resolution for the TM domain further, the IR will be reconstituted into nanodisc, which may stabilize the TM domain by lipid-TM interaction. Nanobodies have been widely used in the structural studies of GPCRs for the stabilization of this dynamic signaling membrane protein. Likewise, nanobodies can be used to stabilize the TM domain of IR through the specific binding to the flexible linker connecting to the TM. The recently developed yeast surface display technology will be exploited to screen such nanobodies that can stabilize the TM and kinase conformation of IR in the S597/S597 analogs bound, active state.

### Example 11. Future Methods for Designing a Pan Specific Mimetic Capable of Activating Known Disease Mutant IRs and Native IR

To date only native amino acids have been implemented in the insulin mimetics, and certain non-native amino acids may provide enhanced potency and or improved pharmacokinetics. Such modifications will be pursued if it is discovered that certain mimetics are not pan-specific for severe insulin resistance mutants or do not fully activate the native IR. These will be synthesized using the methods already developed by simply using protected non-native amino acids. Commercially available D-configuration amino acids as well as side-chain homologs, N-methyl amino acids as well as alternative side chain functionalities such as citrulline and napthylalanine will be utilized. Inclusion of these amino acids is expected to occur during the refinement period once a more complete SAR has been developed following several rounds of optimization.

a. Optimizing the Linker

The Examples above identified a truncated analog that provided enhanced stabilization of the T-state IR complex. The truncated analog involved a deletion in the linker region between the components-1 and -2 interacting helices and demonstrated the role of the linker in complex stabilization. To optimize this linker region, several strategies will be employed. First, the importance of the disulfide bridge in stabilizing a productive T-state of IR will be determined. To test the importance of this linkage, the two cysteines with serine will be replaced and, in addition, salt-bridge analogs will be constructed by replacement of the cysteines with lysine and aspartate in both polarities (i.e., Cys11Lys:Cys18Asp and Cys11Asp:Cys18Lys). These analogs will provide key insights into the role of linker rigidity on T-state stabilization. Secondly, the disulfide bond pattern will be altered to either reduce or expand the intervening residues within the linker. The first example of this was the S597-ΔGly that showed improved stabilization of the T-state by cryo-EM. Further reduction of the linker region will be explored as well as expansion of the linker region by the addition of Gly residues.

b. Maximizing Affinity at Components-1 and -2

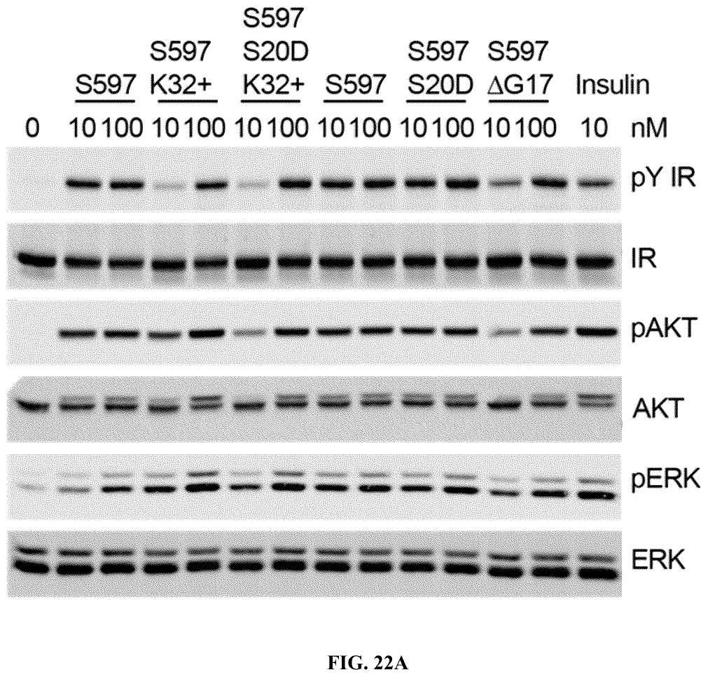

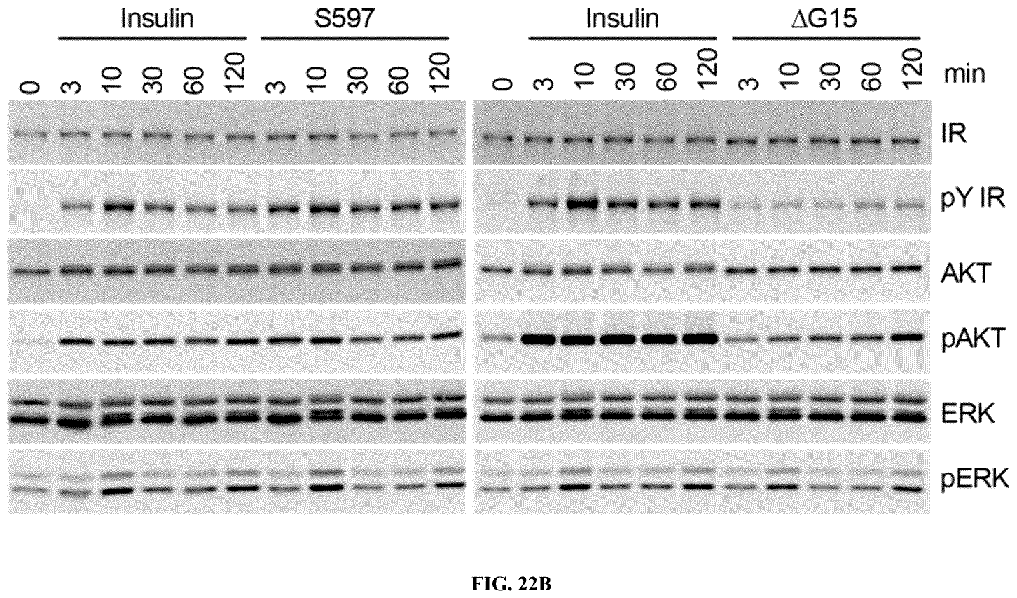

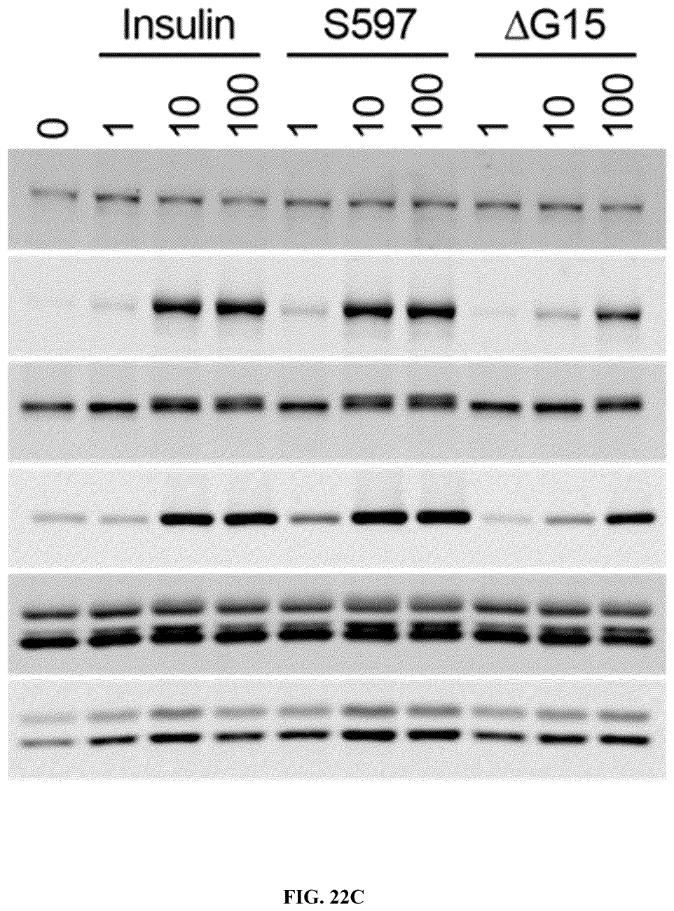

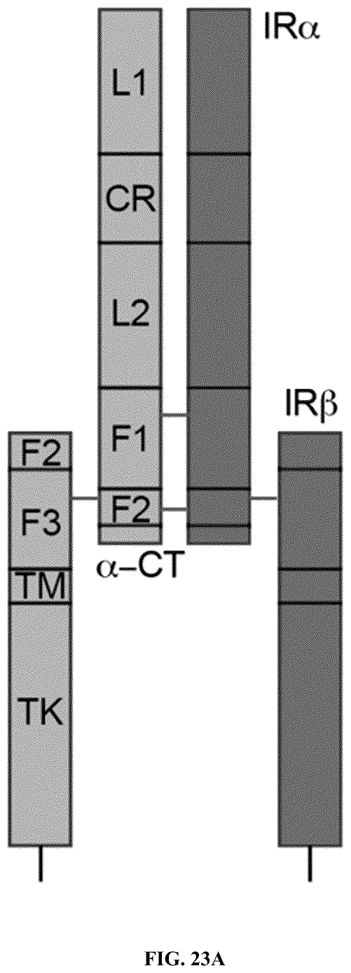

The primary interactions at components-1 and -2 are mediated by hydrophobic amino acid contacts. The component-1 interaction with the L1 domain has a buried surface area of 454 Å² and a calculated ΔG of −9.3 Kcal/mol. The component-2 interaction with the opposite FnIII-1 protomer has a buried surface area 604 Å² and a calculated ΔG −8.1 Kcal/mol. These values are in agreement with most protein-protein interactions and both interactions have almost no hydrogen bonding or salt-bridge interactions. Based upon the current structure, optimizing the hydrophobic packing interactions, while feasible, may not dramatically improve affinity. There are several available sites for potential mutation that could create favorable salt-bridge interactions. Ser20 of component-1 is located near a series of charged residues including Arg118 and His144 of the IR. Structural modelling of the side chain as either an Asp or Glu creates a favorable salt-bridge interaction at this site (FIGS. 21A-22C). Similarly, Ser1 located in component-2 is proximal to Arg479 of the IR and replacement of this Ser with charged residues such as Glu and Asp may form a favorable salt-bridge interaction. The S597-S20D was already produced and tested, which demonstrated more insulin like activity, vide supra, FIGS. 6 and 8-9, validating this structure-based approach despite the lower resolution cryo-EM structure from which these modifications were designed. Studies with the S597-S20E as well as the S597-SID and S597-S1E analog are priorities to understand the importance of this interaction.

c. Expansion of the Component-2 Binding Interaction Site

The component-2 binding site resides on the FnIII-1 domain of the adjacent protomer and has the potential for additional interactions based upon our preliminary S597/IR structural findings. In particular, adding residues to extend the helix and create further surface interactions with the FnIII-1 domain could substantially enhance affinity. This region of the FnIII-1 domain contains a charged residue Asp183, a polar residue Ser181, and an aromatic Phe 182.

d. Gene Combining Mimetic Mutations and SAR

All of the design strategies above will be performed individually and tested for activity at IR and IGF1R as described in AIM1. Those demonstrating favorable activities will be subjected to structural analysis by cryo-EM as described above, and combinatorial mutations will be introduced based upon the subsequent structural data in an effort to maximize affinity and selectivity.

e. Long-Acting Mimetics

The preliminary results demonstrate that an insulin mimetic can effectively recapitulate native insulin signaling and could be effective treatments for rare insulin resistance diseases.

Additionally, it is known that such peptide mimetics are potentially suitable for fatty acid modifications to achieve long-acting activity as has been demonstrated for the GLP-1 agonist94 and GIP/GLP-1 dual agonist. A procedure has been optimized for making such long-acting analogs by incorporating a Boc-His (Trt) at His1 and a Fmoc-Lys (Mtt) (Mtt=4-methyltrityl) at Lys25. After completion of the synthesis the Lys (Mtt) group was removed by two 45-minute treatments of 30% hexafluoroisopropanol/dichloromethane and a single-step coupling of the commercially available preformed side-chain using DIC/HOBt activation. Once several optimized mimetics have been identified, long-acting versions will be produced for subsequent mouse studies.

### REFERENCES

Angelidi, A. M., Filippaios, A. & Mantzoros, C. S. Syndromes of Severe Insulin Resistance. J Clin Invest 131, doi: 10.1172/JCI142245 (2021).
Ardon, O., Procter, M., Tvrdik, T., Longo, N. & Mao, R. Sequencing analysis of insulin receptor defects and detection of two novel mutations in INSR gene. Mol Genet Metab Rep 1, 71-84, doi: 10.1016/j.ymgmr.2013.12.006 (2014).
Bailyes, E. M. et al. Insulin receptor/IGF-I receptor hybrids are widely distributed in mammalian tissues: quantification of individual receptor species by selective immunoprecipitation and immunoblotting. Biochem J 327 (Pt 1), 209-215, doi: 10.1042/bj3270209 (1997).
Bamborschke, D. et al. Ultra-rapid emergency genomic diagnosis of Donahue syndrome in a preterm infant within 17 hours. American journal of medical genetics. Part A 185, 90-96, doi: 10.1002/ajmg.a.61917 (2021).
Bedinger, D. H., Goldfine, I. D., Corbin, J. A., Roell, M. K. & Adams, S. H. Differential pathway coupling of the activated insulin receptor drives signaling selectivity by XMetA, an allosteric partial agonist antibody. J Pharmacol Exp Ther 353, 35-43, doi: 10.1124/jpet.114.221309 (2015).
Boucher, J., Kleinridders, A. & Kahn, C. R. Insulin receptor signaling in normal and insulin-resistant states. Cold Spring Harb Perspect Biol 6, doi: 10.1101/cshperspect.a009191 (2014).
Brandt, S. J., Mayer, J. P., Ford, J., Gelfanov, V. M. & DiMarchi, R. D. Controlled intramolecular antagonism as a regulator of insulin receptor maximal activity. Peptides 100, 18-23, doi: 10.1016/j.peptides.2017.11.022 (2018).
Brierley, G. V. et al. Anti-Insulin Receptor Antibodies Improve Hyperglycemia in a Mouse Model of Human Insulin Receptoropathy. Diabetes 69, 2481-2489, doi: 10.2337/db20-0345 (2020).
Brierley, G. V., Siddle, K. & Semple, R. K. Evaluation of anti-insulin receptor antibodies as potential novel therapies for human insulin receptoropathy using cell culture models. Diabetologia 61, 1662-1675, doi: 10.1007/s00125-018-4606-2 (2018).
Bruning, J. C. et al. A muscle-specific insulin receptor knockout exhibits features of the metabolic syndrome of NIDDM without altering glucose tolerance. Mol Cell 2, 559-569, doi: 10.1016/s1097-2765 (00) 80155-0 (1998).
Burant, C. F., Treutelaar, M. K., Allen, K. D., Sens, D. A. & Buse, M. G. Comparison of insulin and insulin-like growth factor I receptors from rat skeletal muscle and L-6 myocytes. Biochem Biophys Res Commun 147, 100-107, doi: 10.1016/s0006-291x(87) 80092-x (1987).
Caro, J. F. et al. Insulin-like growth factor I binding in hepatocytes from human liver, human hepatoma, and normal, regenerating, and fetal rat liver. J Clin Invest 81, 976-981, doi: 10.1172/JCI113451 (1988).
Choi, E. et al. Mitotic regulators and the SHP2-MAPK pathway promote IR endocytosis and feedback regulation of insulin signaling. Nat Commun 10, 1473, doi: 10.1038/s41467-019-09318-3 (2019).
Choi, E., Zhang, X., Xing, C. & Yu, H. Mitotic Checkpoint Regulators Control Insulin Signaling and Metabolic Homeostasis. Cell 166, 567-581, doi: 10.1016/j.cell.2016.05.074 (2016).
Cochran, E. et al. Efficacy of recombinant methionyl human leptin therapy for the extreme insulin resistance of the Rabson-Mendenhall syndrome. J Clin Endocrinol Metab 89, 1548-1554, doi: 10.1210/jc.2003-031952 (2004).
Croll, T. I. et al. Higher-Resolution Structure of the Human Insulin Receptor Ectodomain: Multi-Modal Inclusion of the Insert Domain. Structure 24, 469-476, doi: 10.1016/j.str.2015.12.014 (2016).
Donohue, W. L. & Uchida, I. Leprechaunism: a euphemism for a rare familial disorder. J Pediatr 45, 505-519, doi: 10.1016/s0022-3476 (54) 80113-2 (1954).
Dutta, D., Maisnam, I., Ghosh, S., Mukhopadhyay, S. & Chowdhury, S. Syndrome of extreme insulin resistance (Rabson-Mendenhall phenotype) with atrial septal defect: clinical presentation and treatment outcomes. J Clin Res Pediatr Endocrinol 5, 58-61, doi: 10.4274/Jcrpe.857 (2013).
Ebina, Y. et al. The human insulin receptor cDNA: the structural basis for hormone-activated transmembrane signalling. Cell 40, 747-758, doi: 10.1016/0092-8674 (85) 90334-4 (1985).
Emsley, P., Lohkamp, B., Scott, W. G. & Cowtan, K. Features and development of Coot. Acta Crystallogr D Biol Crystallogr 66, 486-501, doi: 10.1107/S0907444910007493 (2010).
Frikke-Schmidt, H. et al. Treatment of diabetic rats with insulin or a synthetic insulin receptor agonist peptide leads to divergent metabolic responses. Diabetes 64, 1057-1066, doi: 10.2337/db14-0914 (2015).
Grasso, V. et al. Six cases with severe insulin resistance (SIR) associated with mutations of insulin receptor: Is a Bartter-like syndrome a feature of congenital SIR? Acta Diabetol 50, 951-957, doi: 10.1007/s00592-013-0490-x (2013).
Gutmann, T. et al. Cryo-EM structure of the complete and ligand-saturated insulin receptor ectodomain. J Cell Biol 219, doi: 10.1083/jcb.201907210 (2020).
Gutmann, T., Kim, K. H., Grzybek, M., Walz, T. & Coskun, U. Visualization of ligand-induced transmembrane signaling in the full-length human insulin receptor. J Cell Biol 217, 1643-1649, doi: 10.1083/jcb.201711047 (2018).
Haeusler, R. A., McGraw, T. E. & Accili, D. Biochemical and cellular properties of insulin receptor signalling. Nat Rev Mol Cell Biol 19, 31-44, doi: 10.1038/nrm.2017.89 (2018).
Hart, L. M. et al. An insulin receptor mutant (Asp707-->Ala), involved in leprechaunism, is processed and transported to the cell surface but unable to bind insulin. J Biol Chem 271, 18719-18724, doi: 10.1074/jbc.271.31.18719 (1996).
Jensen, M., Hansen, B., De Meyts, P., Schaffer, L. & Urso, B. Activation of the insulin receptor by insulin and a synthetic peptide leads to divergent metabolic and mitogenic signaling and responses. J Biol Chem 282, 35179-35186, doi: 10.1074/jbc.M704599200 (2007).
Kadowaki, T. et al. Five mutant alleles of the insulin receptor gene in patients with genetic forms of insulin resistance. J Clin Invest 86, 254-264, doi: 10.1172/JCI114693 (1990).
Kadowaki, T. et al. Two mutant alleles of the insulin receptor gene in a patient with extreme insulin resistance. Science 240, 787-790, doi: 10.1126/science.2834824 (1988).
Kadowaki, T., Kadowaki, H., Accili, D. & Taylor, S. I. Substitution of lysine for asparagine at position 15 in the alpha-subunit of the human insulin receptor. A mutation that impairs transport of receptors to the cell surface and decreases the affinity of insulin binding. J Biol Chem 265, 19143-19150 (1990).
Kanter, J. E. et al. A Novel Strategy to Prevent Advanced Atherosclerosis and Lower Blood Glucose in a Mouse Model of Metabolic Syndrome. Diabetes 67, 946-959, doi: 10.2337/db17-0744 (2018).
Kineman, R. D., Del Rio-Moreno, M. & Sarmento-Cabral, A. 40 YEARS of IGF1: Understanding the tissue-specific roles of IGF1/IGF1R in regulating metabolism using the Cre/loxP system. J Mol Endocrinol 61, T187-T198, doi: 10.1530/JME-18-0076 (2018).
Kitamoto, T., Kuo, T., Okabe, A., Kaneda, A. & Accili, D. An integrative transcriptional logic model of hepatic insulin resistance. Proc Natl Acad Sci USA 118, doi: 10.1073/pnas.2102222118 (2021).
Krook, A., Soos, M. A., Kumar, S., Siddle, K. & O'Rahilly, S. Functional activation of mutant human insulin receptor by monoclonal antibody. Lancet 347, 1586-1590, doi: 10.1016/s0140-6736 (96) 91076-4 (1996).
Lawrence, C. F. et al. Insulin Mimetic Peptide Disrupts the Primary Binding Site of the Insulin Receptor. J Biol Chem 291, 15473-15481, doi: 10.1074/jbc.M116.732180 (2016).
Lawrence, M. C. Understanding insulin and its receptor from their three-dimensional structures. Mol Metab, 101255, doi: 10.1016/j.molmet.2021.101255 (2021).
Li, J. et al. Synergistic activation of the insulin receptor via two distinct sites. Nature Structural & Molecular Biology, doi: 10.1038/s41594-022-00750-6 (2022).
Li, J., Choi, E., Yu, H. & Bai, X. C. Structural basis of the activation of type 1 insulin-like growth factor receptor. Nat Commun 10, 4567, doi: 10.1038/s41467-019-12564-0 (2019).
Liebschner, D. et al. Macromolecular structure determination using X-rays, neutrons and electrons: recent developments in Phenix. Acta Crystallogr D Struct Biol 75, 861-877, doi: 10.1107/S2059798319011471 (2019).
Lou, M. et al. The first three domains of the insulin receptor differ structurally from the insulin-like growth factor 1 receptor in the regions governing ligand specificity. Proc Natl Acad Sci USA 103, 12429-12434, doi: 10.1073/pnas.0605395103 (2006).
Mastronarde, D. N. Automated electron microscope tomography using robust prediction of specimen movements. J Struct Biol 152, 36-51, doi: 10.1016/j.jsb.2005.07.007 (2005).
McKern, N. M. et al. Structure of the insulin receptor ectodomain reveals a folded-over conformation. Nature 443, 218-221, doi: 10.1038/nature05106 (2006).
Melvin, A., O'Rahilly, S. & Savage, D. B. Genetic syndromes of severe insulin resistance. Curr Opin Genet Dev 50, 60-67, doi: 10.1016/j.gde.2018.02.002 (2018).
Menting, J. G. et al. How insulin engages its primary binding site on the insulin receptor. Nature 493, 241-245, doi: 10.1038/nature11781 (2013).
Michael, M. D. et al. Loss of insulin signaling in hepatocytes leads to severe insulin resistance and progressive hepatic dysfunction. Mol Cell 6, 87-97 (2000).
Musso, C. et al. Clinical course of genetic diseases of the insulin receptor (type A and Rabson-Mendenhall syndromes): a 30-year prospective. Medicine (Baltimore) 83, 209-222, doi: 10.1097/01.md.0000133625.73570.54 (2004).
O'Brien, R. M., Soos, M. A. & Siddle, K. Monoclonal antibodies to the insulin receptor stimulate the intrinsic tyrosine kinase activity by cross-linking receptor molecules. EMBO J 6, 4003-4010, doi: 10.1002/j.1460-2075.1987.tb02743.x (1987).
Petersen, M. C. & Shulman, G. I. Mechanisms of Insulin Action and Insulin Resistance. Physiol Rev 98, 2133-2223, doi: 10.1152/physrev.00063.2017 (2018).
Pettersen, E. F. et al. UCSF Chimera—a visualization system for exploratory research and analysis. J Comput Chem 25, 1605-1612, doi: 10.1002/jcc.20084 (2004).
Pillutla, R. C. et al. Peptides identify the critical hotspots involved in the biological activation of the insulin receptor. J Biol Chem 277, 22590-22594, doi: 10.1074/jbc.M202119200 (2002).
Plamper, M., Gohlke, B., Schreiner, F. & Woelfle, J. Mecasermin in Insulin Receptor-Related Severe Insulin Resistance Syndromes: Case Report and Review of the Literature. Int J Mol Sci 19, doi: 10.3390/ijms 19051268 (2018).
Quin, J. D. et al. Acute response to recombinant insulin-like growth factor I in a patient with Mendenhall's syndrome. N Engl J Med 323, 1425-1426, doi: 10.1056/NEJM199011153232016 (1990).
Russo, L. et al. Liver-specific reconstitution of CEACAM1 reverses the metabolic abnormalities caused by its global deletion in male mice. Diabetologia 60, 2463-2474, doi: 10.1007/s00125-017-4432-y (2017).
Scapin, G. et al. Structure of the insulin receptor-insulin complex by single-particle cryo-EM analysis. Nature 556, 122-125, doi: 10.1038/nature26153 (2018).
Schaffer, L. et al. Assembly of high-affinity insulin receptor agonists and antagonists from peptide building blocks. Proc Natl Acad Sci USA 100, 4435-4439, doi: 10.1073/pnas.0830026100 (2003).
Semple, R. K., Savage, D. B., Cochran, E. K., Gorden, P. & O'Rahilly, S. Genetic syndromes of severe insulin resistance. Endocr Rev 32, 498-514, doi: 10.1210/er.2010-0020 (2011).
Semple, R. K., Williams, R. M. & Dunger, D. B. What is the best management strategy for patients with severe insulin resistance? Clin Endocrinol (Oxf) 73, 286-290, doi: 10.1111/j.1365-2265.2010.03810.x (2010).
Shao, W. & Espenshade, P. J. Expanding roles for SREBP in metabolism. Cell Metab 16, 414-419, doi: 10.1016/j.cmet.2012.09.002 (2012).
Smith, B. J. et al. Structural resolution of a tandem hormone-binding element in the insulin receptor and its implications for design of peptide agonists. Proc Natl Acad Sci US A 107, 6771-6776, doi: 10.1073/pnas. 1001813107 (2010).
Smith, U. & Gale, E. A. Does diabetes therapy influence the risk of cancer? Diabetologia 52, 1699-1708, doi: 10.1007/s00125-009-1441-5 (2009).
Soos, M. A. et al. Monoclonal antibodies reacting with multiple epitopes on the human insulin receptor. Biochem J 235, 199-208, doi: 10.1042/bj2350199 (1986).
Uchikawa, E., Choi, E., Shang, G., Yu, H. & Bai, X. C. Activation mechanism of the insulin receptor revealed by cryo-EM structure of the fully liganded receptor-ligand complex. Elife 8, doi: 10.7554/eLife.48630 (2019).
Ullrich, A. et al. Insulin-like growth factor I receptor primary structure: comparison with insulin receptor suggests structural determinants that define functional specificity. EMBO J 5, 2503-2512 (1986).
Weis, F. et al. The signalling conformation of the insulin receptor ectodomain. Nat Commun 9, 4420, doi: 10.1038/s41467-018-06826-6 (2018).
Wilkie, N. et al. The non-peptidyl fungal metabolite L-783,281 activates TRK neurotrophin receptors. J Neurochem 78, 1135-1145, doi: 10.1046/j.1471-4159.2001.00504.x (2001).
Xiong, X. et al. Symmetric and asymmetric receptor conformation continuum induced by a new insulin. Nat Chem Biol 18, 511-519, doi: 10.1038/s41589-022-00981-0 (2022).
Yoshimasa, Y. et al. Insulin-resistant diabetes due to a point mutation that prevents insulin proreceptor Science 240, 784-787, processing. doi: 10.1126/science.3283938 (1988).
Zhang, B. et al. Discovery of a small molecule insulin mimetic with antidiabetic activity in mice. Science 284, 974-977, doi: 10.1126/science.284.5416.974 (1999).
Zhang, K. Gctf: Real-time CTF determination and correction. J Struct Biol 193, 1-12, doi: 10.1016/j.jsb.2015.11.003 (2016).
Zheng, S. Q. et al. MotionCor2: anisotropic correction of beam-induced motion for improved cryo-electron microscopy. Nat Methods 14, 331-332, doi: 10.1038/nmeth.4193 (2017) . . . .
Zivanov, J. et al. New tools for automated high-resolution cryo-EM structure determination in RELION-3. Elife 7, doi: 10.7554/eLife.42166 (2018).

We claim:

1. A synthetic peptide of 18-35 amino acid residues in length, wherein the synthetic peptide comprises:
a linker region comprising 4-10 amino acid residues; and
a component 2 region comprising at least 14 amino acid residues;
wherein:
the linker region comprises an amino acid sequence with at least 66% sequence identity to SEQ ID NO. 2; and
the component 2 region comprises an amino acid sequence with at least 85% sequence identity to SEQ ID NO. 1.

2. The synthetic peptide of claim 1 having the amino acid sequence set forth in SEQ ID NO. 45.

3. The synthetic peptide of claim 1, wherein the amino acid sequence of the linker region is selected from the group consisting of: SEQ ID NOs. 3-12.

4. The synthetic peptide of claim 1, wherein the amino acid sequence of the component 2 region has at least 92% sequence identity to SEQ ID NO. 1.

5. The synthetic peptide of claim 4, wherein the amino acid sequence of the component 2 region is selected from the group consisting of SEQ ID NO. 1 and 15-21.

6. The synthetic peptide of claim 1, wherein the synthetic peptide further comprises a component 1 region comprising at least 11 amino acid residues with at least at least 90% sequence identity to SEQ ID NO. 3.

7. The synthetic peptide of claim 6, wherein the amino acid sequence of the component 1 region is set forth in SEQ ID NO. 3 or SEQ ID NO. 22.

8. The synthetic peptide of claim 1, wherein the synthetic peptide is selected from SEQ ID NOs. 23-36.

9. The synthetic peptide of claim 1, wherein the synthetic peptide further comprises an extension of 1-5 amino acid residues at the N terminus.

10. The synthetic peptide of claim 1, wherein the synthetic peptide further comprises a lipidation modification.

11. The synthetic peptide of claim 10, wherein the synthetic peptide is SEQ ID NO. 39.

12. The synthetic peptide of claim 10, wherein at least one glycine, cysteine, or serine residue of the synthetic peptide has the lipidation modification.

13. The synthetic peptide of claim 12, wherein the synthetic peptide is selected from SEQ ID NOs. 40-44.

14. A therapeutic composition comprising:
insulin or analog thereof; and
the synthetic peptide of claim 1.

15. A method of treating insulin resistance in a subject, the method comprises administering to the subject the synthetic peptide of claim 1.

16. The method of claim 15, wherein the subject expresses insulin receptor with site-1 binding-deficient mutations or insulin-desensitized insulin receptor.

17. A method of reducing blood glucose level in a subject, the method comprising administering to the subject the synthetic peptide of claim 1.

18. A method of treating diabetes in a subject, the method comprising administering to the subject the synthetic peptide of claim 1.

19. The method of claim 18, further comprising administering to the subject insulin or analog thereof.

20. The method of claim 18, wherein the subject is diagnosed with cancer.


SEQUENCE LISTING (52 sequences)

SEQ ID NO: 1 - protein, 14 aa, synthetic construct
SLEEEWAQIECEVY

SEQ ID NO: 2 - protein, 6 aa, synthetic construct
GRGCPS

SEQ ID NO: 3 - protein, 11 aa, synthetic construct
ESFYDWFERQL

SEQ ID NO: 4 - protein, 6 aa, synthetic construct
GRGSPS

SEQ ID NO: 5 - protein, 6 aa, synthetic construct
GRGDPS

SEQ ID NO: 6 - protein, 6 aa, synthetic construct
GRGKPS

SEQ ID NO: 7 - protein, 5 aa, synthetic construct
GRCPS

SEQ ID NO: 8 - protein, 4 aa, synthetic construct
RCPS

SEQ ID NO: 9 - protein, 6 aa, synthetic construct
GRGCPD

SEQ ID NO: 10 - protein, 6 aa, synthetic construct
GRGCPE

SEQ ID NO: 11 - protein, 6 aa, synthetic construct
GRGCPQ

SEQ ID NO: 12 - protein, 6 aa, synthetic construct
GRKCPS

SEQ ID NO: 13 - protein, 10 aa, synthetic construct
GRGCPSGSLD

SEQ ID NO: 14 - protein, 5 aa, synthetic construct
SGSLD

SEQ ID NO: 15 - protein, 14 aa, synthetic construct
DLEEEWAQIECEVY

SEQ ID NO: 16 - protein, 14 aa, synthetic construct
ELEEEWAQIECEVY

SEQ ID NO: 17 - protein, 14 aa, synthetic construct
QLEEEWAQIESEVY

SEQ ID NO: 18 - protein, 14 aa, synthetic construct
SLEEEAAQIECEVY

SEQ ID NO: 19 - protein, 14 aa, synthetic construct
SLEEEWAQIESEVY

SEQ ID NO: 20 - protein, 14 aa, synthetic construct
SLEEEWAQIEKEVY

SEQ ID NO: 21 - protein, 14 aa, synthetic construct
SLEEEWAQIEDEVY

SEQ ID NO: 22 - protein, 10 aa, synthetic construct
ESFYDWARQL

SEQ ID NO: 23 - protein, 31 aa, synthetic construct
SLEEEWAQIECEVYGRGCPSESFYDWFERQL

SEQ ID NO: 24 - protein, 31 aa, synthetic construct
DLEEEWAQIECEVYGRGCPSESFYDWFEROL

SEQ ID NO: 25 - protein, 31 aa, synthetic construct
ELEEEWAQIECEVYGRGCPSESFYDWFERQL

SEQ ID NO: 26 - protein, 31 aa, synthetic construct
QLEEEWAQIESEVYGRGSPSESFYDWFERQL

SEQ ID NO: 27 - protein, 31 aa, synthetic construct
SLEEEAAQIECEVYGRGCPSESFYDWFERQL

SEQ ID NO: 28 - protein, 31 aa, synthetic construct
SLEEEWAQIESEVYGRGSPSESFYDWFERQL

SEQ ID NO: 29 - protein, 31 aa, synthetic construct
SLEEEWAQIEKEVYGRGDPSESFYDWFERQL

SEQ ID NO: 30 - protein, 31 aa, synthetic construct
SLEEEWAQIEDEVYGRGKPSESFYDWFERQL

SEQ ID NO: 31 - protein, 30 aa, synthetic construct
SLEEEWAQIECEVYGRCPSESFYDWFERQL

SEQ ID NO: 32 - protein, 29 aa, synthetic construct
SLEEEWAQIECEVYRCPSESFYDWFERQL

SEQ ID NO: 33 - protein, 31 aa, synthetic construct
SLEEEWAQIECEVYGRGCPDESFYDWEEROL

SEQ ID NO: 34 - protein, 31 aa, synthetic construct
SLEEEWAQIECEVYGRGCPEESFYDWEEROL

SEQ ID NO: 35 - protein, 31 aa, synthetic construct
SLEEEWAQIECEVYGRGCPQESFYDWFEROL

SEQ ID NO: 36 - protein, 30 aa, synthetic construct
SLEEEWAQIECEVYGRGCPSESFYDWARQL

SEQ ID NO: 37 - protein, 31 aa, synthetic construct; identical to SEQ ID NO: 23
  SITE 1: note = N-terminus extended by three amino acid residues

SEQ ID NO: 38 - protein, 31 aa, synthetic construct; identical to SEQ ID NO: 23
  SITE 1: note = Lipidation of N-terminus

SEQ ID NO: 39 - protein, 31 aa, synthetic construct; identical to SEQ ID NO: 23
  SITE 31: note = Lipidation of C-terminus

SEQ ID NO: 40 - protein, 31 aa, synthetic construct; identical to SEQ ID NO: 23
  SITE 18: note = Lipidation of cysteine at residue 18

SEQ ID NO: 41 - protein, 31 aa, synthetic construct; identical to SEQ ID NO: 23
  SITE 1: note = Lipidation of serine at residue 1

SEQ ID NO: 42 - protein, 30 aa, synthetic construct; identical to SEQ ID NO: 31
  SITE 17: note = Lipidation of lysine at residue 17

SEQ ID NO: 43 - protein, 31 aa, synthetic construct; identical to SEQ ID NO: 28
  SITE 11: note = Lipidation of serine at residue 11
  SITE 18: note = Lipidation of serine at residue 18

SEQ ID NO: 44 - protein, 31 aa, synthetic construct; identical to SEQ ID NO: 29
  SITE 11: note = Lipidation of aspartic acid at residue 11
  SITE 18: note = Lipidation of lysine at residue 18

SEQ ID NO: 45 - protein, 31 aa, synthetic construct; identical to SEQ ID NO: 33
  SITE 31: note = Lipidation of C-terminus
  SITE 20: note = Lipidation of aspartic acid at residue 20

SEQ ID NO: 46 - protein, 20 aa, synthetic construct
SLEEEWAQIECEVYGRGCPS

SEQ ID NO: 47 - protein, 35 aa, synthetic construct
SLEEEWAQIECEVYGRGCPSGSLDESFYDWFERQL

SEQ ID NO: 48 - protein, 16 aa, synthetic construct
SGSLDESFYDWFERQL

SEQ ID NO: 49 - protein, 15 aa, synthetic construct
GCPSESFYDWFERQL

SEQ ID NO: 50 - protein, 15 aa, synthetic construct
GSLDESFYDWFERQL

SEQ ID NO: 51 - protein, 15 aa, synthetic construct
GCPDESFYDWFERQL

SEQ ID NO: 52 - protein, 15 aa, synthetic construct
ELEESSFRKTFEDYL